(19) United States
(12) Patent Application Publication
(10) Pub. No.: US 2021/0207217 A1
(43) Pub. Date: Jul. 8, 2021

(54) DNA METHYLATION BASED BIOMARKERS FOR IRRITABLE BOWEL SYNDROME AND INFLAMMATORY BOWEL DISEASE

(71) Applicant: THE REGENTS OF THE UNIVERSITY OF CALIFORNIA, OAKLAND, CA (US)

(72) Inventors: Lin Chang, MANHATTAN BEACH, CA (US); Swapna Joshi, CULVER CITY, CA (US)

(73) Assignee: THE REGENTS OF THE UNIVERSITY OF CALIFORNIA, OAKLAND, CA (US)

(21) Appl. No.: 17/059,747

(22) PCT Filed: May 31, 2019
(86) PCT No.: PCT/US19/35039

§ 371 Date: Nov. 30, 2020

Related U.S. Application Data
(60) Provisional application No. 62/678,618, filed on May 31, 2018.

(51) Int. Cl. C12Q 1/6883

(52) U.S. Cl. CPC C12Q 1/6883; C12Q 2600/154

(57) ABSTRACT

Methods, kits, devices, and materials described herein provide blood-based diagnostic, prognostic, and treatment-monitoring biomarkers for IBS and IBD. These biomarkers can be used to distinguish IBS and/or IBD patients from healthy controls, for example, as well as to distinguish between IBS and IBD or other related disorders.

This application claims benefit of U.S. provisional patent application No. 62/678,618, filed May 31, 2018, the entire contents of which are incorporated by reference into this application.

### ACKNOWLEDGEMENT OF GOVERNMENT SUPPORT

This invention was made with government support under Grant Numbers DK064539 and DK104078, awarded by the National Institutes of Health. The government has certain rights in the invention.

### BACKGROUND OF THE INVENTION

Irritable bowel syndrome (IBS) is a stress-sensitive, chronic gastrointestinal (GI) disorder characterized by chronic abdominal pain associated with diarrhea and/or constipation. IBS occurs in children and adults and has a female predominance. It affects up to 11% of the US population but is prevalent worldwide. Annually, IBS accounts for 3.1 million ambulatory care visits, 5.9 million prescriptions and has a total direct and indirect cost exceeding $20 billion. Most IBS patients have seen at least three physicians and undergo multiple expensive and invasive tests before a diagnosis of IBS as IBS is often considered a diagnosis of exclusion. IBS is currently diagnosed based on symptom-based criteria due to the lack of a diagnostic biomarker.

There remains a need for markers that can identify patients with IBS, particularly for markers that can distinguish IBS with high specificity and sensitivity. There remains a particular need to distinguish IBS from inflammatory bowel disease (IBD).

### SUMMARY OF THE INVENTION

The methods, kits, devices, and materials described herein provide blood-based diagnostic, prognostic, and treatment-monitoring biomarkers for IBS and IBD. These biomarkers can be used to distinguish IBS and/or IBD patients from healthy controls, for example, as well as to distinguish between IBS and IBD or other related disorders.

Described herein is a method of measuring DNA methylation in a biological sample obtained from a subject. In one embodiment, the method comprises (a) generating an irritable bowel syndrome (IBS)/inflammatory bowel disease (IBD) methylation profile from the biological sample obtained from the subject, wherein the profile comprises at least 50 CpG sites of the IBS/IBD biomarker genes listed in Tables 16, 17, 18, 19, and/or 20. The method further comprises (b) measuring the amount of methylation in the IBS/IBD biomarker genes. The amount of biomarker methylation is used to classify the profile. A profile can be classified as an IBS profile, an IBD, profile, an ulcerative colitis (UC), a Crohn's Disease (CD) profile, or a normal, healthy control (non-IBS/IBD) profile.

In some embodiments, the methylation profile comprises at least 100 of the CpG sites of genes listed in Tables 16-20. In other embodiments, the methylation profile comprises at least 40 of the CpG sites listed in any of Tables 16-20. In some embodiments, 50, 70, 80, 150, 200, 250, 300, 350, 400, or 405 of the CpG sites of the genes listed in Tables 16-20, or up to 450, 500, or all 550 of the CpG sites of the genes listed in Table 20 are included in the methylation profile. In some embodiments, only genes listed in the Tables provided herein are included in the methylation profile. In other embodiments, the methylation profile includes additional genes beyond those listed in the Tables herein. Typically, the methylation sites are CpG sites. In some embodiments, the methylation sites are in a promoter region or associated with a regulatory control element. In some embodiments, generating the IBS/IBD methylation profile comprises preprocessing the biological sample with a kit for measuring the amount of methylation on all CpG sites.

In some embodiments, the subject has manifested clinical symptoms associated with IBS. In some embodiments, the subject has manifested clinical symptoms associated with IBD. In some embodiments, the subject has manifested symptoms associated with both IBS and IBD, and the method is used to determine whether the subject has IBS, IBD, or both. In some embodiments, the IBD is ulcerative colitis (UC). In some embodiments, the IBD is Crohn's Disease (CD).

In some embodiments, the IBS/IBD biomarker genes are selected from genes differentially methylated between IBS and healthy controls and listed in Table 16 or 20. In some embodiments, the IBS/IBD biomarker genes are selected from genes differentially methylated between ulcerative colitis (UC) and healthy controls as shown in Table 17. In some embodiments, the IBS/IBD biomarker genes are selected from genes differentially methylated between Crohn's Disease (CD) and healthy controls and listed in Table 18. In some embodiments, the IBS/IBD biomarker genes are selected from genes differentially methylated between IBS and IBD and listed in Table 19.

In some embodiments, a computer algorithm determines a conditional probability of IBS based on the profile. In some embodiments, the determination of the presence of IBS is achieved by following the steps illustrated in FIG. 9. These steps can optionally be performed with the assistance of a processor. In some embodiments, each potential methylation site is weighted equally. In some embodiments, certain potential methylation sites are given more weight in the classification of the profile. The selection and/or weighting of potential methylation sites can be based on gene traits and/or on location within a gene, such as near a promoter or regulatory element. Such selection can also be based on modules identified herein, wherein more than one gene is identified as belonging to a module consisting of highly correlated genes, such that one may select one or more genes representative of a given module, or of each module. Also identified herein are some genes that do not appear to be connected to other genes, and thus, one may select most or all members of this category of IBS/IBD biomarker genes.

In some embodiments, the method further comprises calculating the percentage of CpG sites on the IBS/IBD biomarker genes that are methylated, wherein a percentage of CpG sites methylated in excess of 40% is indicative of IBS or IBD. In some embodiments, the percentage of CpG sites that show increased methylation is over 50%, 60%, or 70%. In some embodiments, the amount of biomarker methylation is greater than 54% of CpG sites on the IBS/IBD biomarker genes. In some embodiments, the percentage of CpG sites that are methylated in healthy controls is less than 30%. In some embodiments, the percentage of CpG sites that are methylated in healthy controls is less than 20%.

In some embodiments, the method further comprises (c) classifying the profile as: (i) an IBS profile if at least 50% of the CpG sited on the genes listed in Table 16 or 20 are methylated; (ii) a UC profile if at least 50% of the CpG sited on the genes listed in Table 17 are methylated; and/or (iii) a CD profile if at least 50% of the CpG sited on the genes listed in Table 18 are methylated. In this context, the methylation of sites refers to a hyper-methylation compared to healthy controls. The method further comprises (d) administering treatment for IBS, UC, or CD, in accordance with the classified profile. Alternatively, the classifying of the profile as IBS, UC, CD, or non-IBS/IBD (or as normal or healthy) is based on a lower or higher percentage as noted herein, or is based on an algorithm or on machine learning or on the process illustrated in FIG. 9.

Also provided is a method of treating IBS. In one embodiment, the method comprises performing one of the methods described above, and administering treatment for IBS if the methylation profile is classified as an IBS profile. In some embodiments, the treatment comprises administering rifaximin, loperamide, eluxadoline, alosetron, lubiprostone, linaclotide, plecanatide, a laxative, an antihistamine, an antispasmodic, a neuromodulator, dietary therapy, or behavioral therapy.

Additionally provided is a method of screening for IBS, UC, or CD in a subject. In one embodiment, the method comprises (a) generating an IBS/IBD methylation profile from a biological sample obtained from the subject, wherein the profile comprises at least 50 of the IBS/IBD biomarker genes (or CpG sites) listed in Tables 16-20; and (b) measuring the amount of methylation in the IBS/IBD biomarker genes. The amount of biomarker methylation is used to classify the profile, and a subject is identified as having IBS, UC, or CD based on the profile. In a further embodiment, provided is a method of monitoring progression of or treatment for IBS, UC, or CD in a subject. The method comprises (a) generating an IBS/IBD methylation profile from a biological sample obtained from the subject, wherein the profile comprises at least 50 of the IBS/IBD biomarker genes listed in Tables 16-20; and (b) measuring the amount of methylation in the IBS/IBD biomarker genes. The amount of biomarker methylation is used to classify the profile, and a subject is identified as having IBS, UC, or CD that is progressing, or is responding to treatment, based on the profile. The treatment for the subject can then be modified based on the profile. Such modification of treatment can include, for example, increasing, decreasing, initiating, restarting, or ceasing treatment. The monitoring can be repeated as needed to ensure long term optimization of care.

In some embodiments, the biological sample comprises blood, plasma, serum, saliva, or mucosal tissue. In some embodiments, the sample is peripheral blood mononuclear cells (PBMCs), peripheral blood lymphocytes (PBL), or whole blood.

Additionally provided are kits, devices, and materials for use in carrying out the methods described herein.

### BRIEF DESCRIPTION OF THE DRAWINGS

FIG. 1: This figure shows plots for top 4 correlations between stress-related genes and clinical features of IBS patients, i.e., brain-derived neurotrophic factor (BDNF) vs patient health questionnaire (PHQ-15), histone deacetylase (HDAC4) vs adverse childhood events (ACE score), HDAC4 vs bloating and corticotrophin releasing hormone receptor 2 (CRHR2) vs PHQ-15 for PBMCs on the left and correlations between transient receptor potential cation channel, subfamily V, member 1 (TRPV1) vs perceived stress score (PSS), cannabinoid receptor 1 (CNR1) vs PHQ-15 and FK506 binding protein 4 (FKBP4) vs PHQ-15 in colon samples, on the right. Y-axis label shows probe ID for the differentially methylated CpG site.

FIG. 2: This figure shows receiver operating characteristic (ROC) curve on the left and box plot showing methylation beta value, averaged over the selected biomarkers for IBS and healthy controls (HC). The area under the curve (AUC) for the ROC curve was 0.92.

FIG. 3: Box plots show significant association of methylation-based Clusters in colonic mucosa of IBS patients with abdominal pain (p=0.004) and overall severity (p=0.0002).

FIG. 4: Co-methylation module and trait relationship in IBS. Correlation of co-methylation modules (Y-axis) and IBS endophenotypes (X-axis). The black boxes show significant correlations of interest. Shading represents negative and positive correlations, per density scale shown at right, and the intensity of the shading is proportional to the extent of correlation.

FIG. 5: Starburst plot integrating differentially methylated and differentially expressed genes between A. IBS and healthy controls and B. Cluster 1 compared to Cluster 3. The black dots represent genes with significantly higher methylation and lower expression (p<0.05).

FIG. 6: Receiver operating characteristic (ROC) curve for DNA methylation based biomarkers in PBMCs that discriminate IBS from IBD.

FIG. 7: Schematic of inflammatory mediator of TRP channels' pathway. Green boxes are genes in the pathway and the ones with a red asterisk are differentially methylated between IBS and IBD.

FIG. 8: Weighted gene co-expression network analysis modules.

FIG. 9: Flow chart illustrating one embodiment of the method for assessing DNA methylation profiles associated with IBS and IBD.

### DETAILED DESCRIPTION OF THE INVENTION

The invention provides new methods and tools for blood-based diagnosis of IBS, i.e. differentiating IBS patients from healthy controls (HCs) and other diseases with symptoms that mimic IBS (e.g. celiac disease, IBD, and colon cancer). This discovery shifts the paradigm of diagnosing IBS. Methods and tools are also provided for diagnosing IBD, and for monitoring response to treatment of IBS and IBD.

Using DNA from blood samples, we identified a methylation signature of 550 markers associated with a group of genes which can distinguish IBS patients from HCs (area under the ROC curve (AUC)=0.89, p=0.001, ˜91% positive predictive value (PPV) and ˜79% negative predictive value (NPV)). Furthermore, a panel of 100 markers discriminated IBS from IBD (AUC=1.0, p=1.12e-17, 100% PPV and 100% NPV). We developed a panel of 650 methylation markers that clearly differentiate IBS from HCs and IBD using a blood test.

### Definitions

All scientific and technical terms used in this application have meanings commonly used in the art unless otherwise specified. As used in this application, the following words or phrases have the meanings specified.

The term “nucleic acid” or “polynucleotide” or “oligonucleotide” refers to a sequence of nucleotides, a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

The term “primer,” as used herein, means an oligonucleotide designed to flank a region of DNA to be amplified. In a primer pair, one primer is complementary to nucleotides present on the sense strand at one end of a polynucleotide fragment to be amplified and another primer is complementary to nucleotides present on the antisense strand at the other end of the polynucleotide fragment to be amplified. A primer can have at least about 11 nucleotides, and preferably, at least about 16 nucleotides and no more than about 35 nucleotides. Typically, a primer has at least about 80% sequence identity, preferably at least about 90% sequence identity with a target polynucleotide to which the primer hybridizes.

As used herein, the term “probe” refers to an oligonucleotide, naturally or synthetically produced, via recombinant methods or by PCR amplification, that hybridizes to at least part of another oligonucleotide of interest. A probe can be single-stranded or double-stranded.

As used herein, the term “active fragment” refers to a substantial portion of an oligonucleotide that is capable of performing the same function of specifically hybridizing to a target polynucleotide.

As used herein, “hybridizes,” “hybridizing,” and “hybridization” means that the oligonucleotide forms a noncovalent interaction with the target DNA molecule under standard conditions. Standard hybridizing conditions are those conditions that allow an oligonucleotide probe or primer to hybridize to a target DNA molecule. Such conditions are readily determined for an oligonucleotide probe or primer and the target DNA molecule using techniques well known to those skilled in the art. The nucleotide sequence of a target polynucleotide is generally a sequence complementary to the oligonucleotide primer or probe. The hybridizing oligonucleotide may contain nonhybridizing nucleotides that do not interfere with forming the noncovalent interaction. The nonhybridizing nucleotides of an oligonucleotide primer or probe may be located at an end of the hybridizing oligonucleotide or within the hybridizing oligonucleotide. Thus, an oligonucleotide probe or primer does not have to be complementary to all the nucleotides of the target sequence as long as there is hybridization under standard hybridization conditions.

The term “complement” and “complementary” as used herein, refers to the ability of two DNA molecules to base pair with each other, where an adenine on one DNA molecule will base pair to a guanine on a second DNA molecule and a cytosine on one DNA molecule will base pair to a thymine on a second DNA molecule. Two DNA molecules are complementary to each other when a nucleotide sequence in one DNA molecule can base pair with a nucleotide sequence in a second DNA molecule. For instance, the two DNA molecules 5′-ATGC and 5′-GCAT are complementary, and the complement of the DNA molecule 5′-ATGC is 5′-GCAT. The term complement and complementary also encompasses two DNA molecules where one DNA molecule contains at least one nucleotide that will not base pair to at least one nucleotide present on a second DNA molecule. For instance, the third nucleotide of each of the two DNA molecules 5′-ATTGC and 5′-GCTAT will not base pair, but these two DNA molecules are complementary as defined herein. Typically, two DNA molecules are complementary if they hybridize under the standard conditions referred to above. Typically, two DNA molecules are complementary if they have at least about 80% sequence identity, preferably at least about 90% sequence identity.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, Pa., 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

As used herein, to “prevent” or “protect against” a condition or disease means to hinder, reduce or delay the onset or progression of the condition or disease.

### Methods of the Invention

The invention provides methods for measuring DNA methylation in a biological sample obtained from a subject. Typically, the method comprises: (a) generating an IBS/IBD methylation profile from the biological sample obtained from the subject, wherein the profile comprises a plurality of IBS/IBD biomarker genes having CpG sites; and (b) measuring the amount of methylation in the IBS/IBD biomarker genes; wherein the amount of biomarker methylation is used to classify the profile. A profile can be classified as an IBS profile, an IBD, profile, an ulcerative colitis (UC), a Crohn's Disease (CD) profile, or a normal, healthy control (non-IBS/IBD) profile.

In some embodiments, the methylation profile comprises at least 100 of the genes or CpG sites listed in one or all of Tables 16-20. In other embodiments, the methylation profile comprises at least 40, 50, 70, 80, 150, 200, 250, 300, 350, 400, 450, 500, 505, 550 of the genes or CpG sites listed in Tables 16-20. In some embodiments, the profile comprises 100 CpG sites listed in Tables 17 and 18, and 505 or 550 CpG sites listed in Table 16 or 20 (e.g. up at a total of 650 sites). The Tables herein, such as Tables 16-20, provide annotations of CpG islands connected with a “cg number”. This information is provided by Illumina and can be used to identify the context of the sequences and probes to be used in the methods and assays described herein. One can access a manifest file for additional information via ftp://webdata2:webdata2@ussd-ftp.illumina.com/downloads/ProductFiles/HumanMethylation450/HumanMethylation450_15017482_v1-2.csv or https://support.illumina.com/array/array_kits/infinium_humanmethylation450_beadchip_kit/downloads.html.

In some embodiments, only genes listed in the Tables provided herein are included in the methylation profile. In other embodiments, the methylation profile includes additional genes beyond those listed in the Tables herein. Typically, the methylation sites are CpG sites. In some embodiments, the methylation sites are in a promoter region or associated with a regulatory control element. In some embodiments, generating the IBS/IBD methylation profile comprises preprocessing the biological sample with a kit for measuring the amount of methylation on all CpG sites.

In some embodiments, the subject has manifested clinical symptoms associated with IBS. In some embodiments, the subject has manifested clinical symptoms associated with IBD. In some embodiments, the subject has manifested symptoms associated with both IBS and IBD, and the method is used to determine whether the subject has IBS, IBD, or both. In some embodiments, the IBD is ulcerative colitis (UC). In some embodiments, the IBD is Crohn's Disease (CD).

In some embodiments, the IBS/IBD biomarker genes are selected from genes differentially methylated between IBS and healthy controls and listed in Tables 16-20. In some embodiments, the IBS/IBD biomarker genes are selected from genes differentially methylated between ulcerative colitis (UC) and healthy controls as shown in Table 17. In some embodiments, the IBS/IBD biomarker genes are selected from genes differentially methylated between Crohn's Disease (CD) and healthy controls and listed in Table 18. In some embodiments, the IBS/IBD biomarker genes are selected from genes differentially methylated between IBS and IBD and listed in Table 19.

In some embodiments, generating the IBS methylation profile comprises preprocessing the biological sample with a kit for measuring the amount of methylation on all CpG sites. Methylation can be measured using commercially available kits. In some embodiments, a computer algorithm determines a conditional probability of IBS based on the profile. One example of an algorithm for use in the classifying is a random forest algorithm. In one representative example, the algorithm takes methylation levels of all biomarker probes assessed using a custom chip and uses rules or binning criteria from each decision tree randomly created from a training data set to predict outcome, and stores the predicted outcome. Next, votes for each predicted outcome are calculated. The final prediction is based on the highest voted predicted target. Rules are a series of questions which have binary answers: yes or no. For example, is the methylation level of probe X >0.5? If yes, go to methylation of probe Y. Is it >0.5? If so, go to 3, if the answer is yes, bin it as IBS, if not HC. With 550 probes having different levels of methylation, each sample is trained based on the IBS status, making one sample as one tree and CpG probes as nodes. All samples together in a training set look like a forest. When a new sample is introduced, a tree is constructed based on the rules defined using all the trees in the forest and a decision is made on the basis of resemblance of this tree to other tress in IBS or HC bin.

Random forest classification employs the Bagging method to produce a randomly sampled set of training data for each of the trees. This Random Forests method also semi-randomly selects splitting features (CpG sites with different methylation pattern for IBS vs controls); a random subset of a given size is produced from the space of possible splitting features. The best splitting feature is deterministically (using median) selected from that subset. Random Forest classifies the test sample by simply combining all results from each of the trees in the forest. The method used to combine the results can be as simple as predicting the class obtained from the highest number of trees.

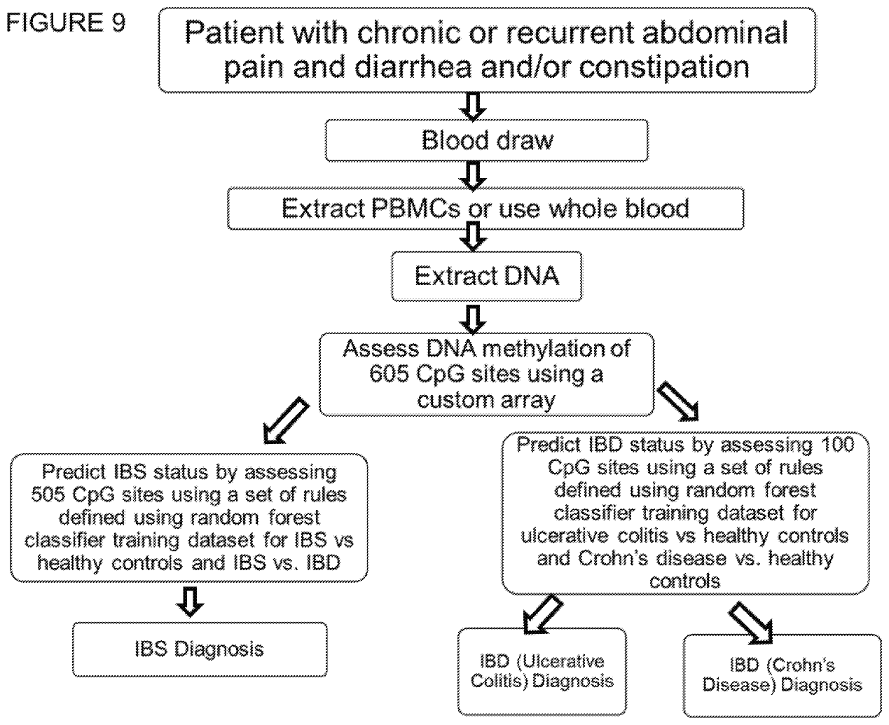

In some embodiments, the determination of the presence of IBS is achieved by following the steps illustrated in FIG. 9. These steps can optionally be performed with the assistance of a processor. In some embodiments, each potential methylation site is weighted equally. In some embodiments, certain potential methylation sites are given more weight in the classification of the profile. The selection and/or weighting of potential methylation sites can be based on gene traits and/or on location within a gene, such as near a promoter or regulatory element. Such selection can also be based on modules identified herein, wherein more than one gene is identified as belonging to a module consisting of highly correlated genes, such that one may select one or more genes representative of a given module, or of each module. Also identified herein are some genes that do not appear to be connected to other genes, and thus, one may select most or all members of this category of IBS/IBD biomarker genes.

In some embodiments, the method further comprises calculating the percentage of CpG sites on the IBS/IBD biomarker genes that are methylated, wherein a percentage of CpG sites methylated in excess of 40% is indicative of IBS or IBD. In some embodiments, the percentage of CpG sites that show increased methylation is over 50%, 60%, or 70%. In some embodiments, the amount of biomarker methylation is greater than 54% of CpG sites on the IBS/IBD biomarker genes. In some embodiments, the percentage of CpG sites that are methylated in healthy controls is less than 30%. In some embodiments, the percentage of CpG sites that are methylated in healthy controls is less than 20%.

In some embodiments, the method further comprises (c) classifying the profile as: (i) an IBS profile if at least 50% of the CpG sites of the genes listed in Table 16 and/or 20 are methylated; (ii) a UC profile if at least 50% of the CpG sited on the genes listed in Table 17 are methylated; and/or (iii) a CD profile if at least 50% of the CpG sited on the genes listed in Table 18 are methylated. The method further comprises (d) administering treatment for IBS, UC, or CD, in accordance with the classified profile. Alternatively, the classifying of the profile as IBS, UC, CD, or non-IBS/IBD (or as normal or healthy) is based on a lower or higher percentage as noted herein, or is based on an algorithm or on machine learning and/or on the process illustrated in FIG. 9.

FIG. 9 illustrates a representative embodiment of the method. Blood is drawn from a patient who presents with chronic or recurrent abdominal pain and diarrhea and/or constipation. DNA is extracted from PBMCs or whole blood. DNA methylation is assessed at 605 CpG sites using a custom array. 505 CpG sites are used to predict IBS status using a set of rules defined using random forest classifier training dataset for IBS versus health controls and IBS versus IBD, leading to a diagnosis of IBS. The remaining 100 sites are used to predict IBD status using a set of rules defined using random forest classifier training dataset for ulcerative colitis versus healthy controls and Crohn's disease versus healthy controls. This assessment leads to a diagnosis regarding UC or CD.

The invention further provides a method of treating IBS. In one embodiment, the method comprises performing the measuring described herein, and administering treatment for IBS. Representative examples of treatments include, but are not limited to, administering rifaximin, loperamide, eluxadoline, alosetron, lubiprostone, linaclotide, plecanatide, a laxative, an antihistamine, an antispasmodic, a neuromodulator, dietary therapy, or behavioral therapy.

Also provided is a method of screening for IBS and/or IBD, such as UC or CD, in a subject. In one embodiment, the method comprises (a) generating an IBS/IBD methylation profile from a biological sample obtained from the subject, wherein the profile comprises at least 50 of the IBS/IBD biomarker genes listed in Table 16, 17, 18, 19, and/or 20; and (b) measuring the amount of methylation in the IBS/IBD biomarker genes. The amount of biomarker methylation is used to classify the profile, and a subject is identified as having IBS, UC, or CD based on the profile.

In a further embodiment, provided is a method of monitoring progression of or treatment for IBS, UC, or CD in a subject. The method comprises (a) generating an IBS/IBD methylation profile from a biological sample obtained from the subject, wherein the profile comprises at least 50 of the IBS/IBD biomarker genes listed in Tables 16-20; and (b) measuring the amount of methylation in the IBS/IBD biomarker genes. The amount of biomarker methylation is used to classify the profile, and a subject is identified as having IBS, UC, or CD that is progressing, or is responding to treatment, based on the profile. The treatment for the subject can then be modified based on the profile. DNA methylation signatures can be modulated in response to treatment and provide an indication of inflammation and other symptoms (see, e.g., Somineni et al., 2019, Gastroenterology 156:2254-2265). Such modification of treatment can include, for example, increasing, decreasing, initiating, restarting, or ceasing treatment. The monitoring can be repeated as needed to ensure long term optimization of care.

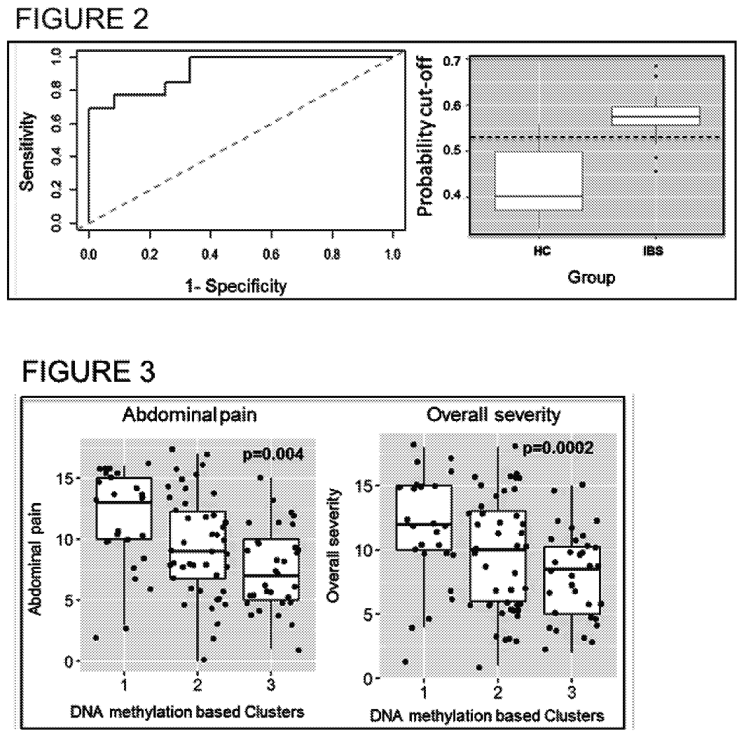

The amount of methylation of the biomarker genes is indicative of IBS/IBD status. In some embodiments, the average methylation of the indicated IBS/IBD biomarker genes provides the amount of biomarker methylation to be used to classify the profile. In some embodiments, the classification is based on whether the combined levels of methylation are greater than 0.54 (the cutoff which best discriminates IBS from healthy controls, wherein a methylated site is assigned a value of 1 and an unmethylated site is assigned a value of 0). For example, if a person's biological sample is tested and the sample has (average) methylation levels on these biomarkers close to 0.65, there is a high probability that she or he is an IBS patient. Conversely, if the levels are close to 0.35, there is 0% chance that the person has IBS (see right panel of FIG. 2).

Subjects are, in some embodiments, adults, and in other embodiments, children.

For use in the methods described herein, representative examples of the sample include, but are not limited to, blood, plasma or serum, saliva, urine, cerebral spinal fluid, milk, cervical secretions, semen, tissue, cell cultures, and other bodily fluids or tissue specimens. In some embodiments, the biological sample comprises blood, plasma, serum, saliva, or mucosal tissue. In some embodiments, the sample is peripheral blood mononuclear cells (PBMCs), peripheral blood lymphocytes (PBL), or whole blood.

### Kits and Assay Standards

The invention provides kits comprising a set of reagents as described herein, such as probes that specifically bind one or more markers of the invention (including genes and their expression products), and optionally, one or more suitable containers containing reagents of the invention. A kit can comprise the materials useful for detecting methylation, including a set of probes, optionally immobilized to an array. A set of probes can include 10 or more probes, 20 or more probes, 50 or more, 100 or more, and up to 100, 200, 300, 400, 500, 600, or more probes.

Reagents include molecules that specifically bind and/or amplify and/or detect one or more markers of the invention. Such molecules can be provided in the form of a microarray, next generation sequencing, or other article of manufacture for use in an assay described herein. One example of a reagent is an antibody or nucleic acid probe that is specific for the marker(s). Another example includes probes (or primers) that selectively identify one or more genotypes described herein. Reagents can optionally include a detectable label. Labels can be fluorescent, luminescent, enzymatic, chromogenic, or radioactive.

Kits of the invention optionally comprise an assay standard or a set of assay standards, either separately or together with other reagents. An assay standard can serve as a normal control by providing a reference level of normal expression for a given marker that is representative of a healthy individual.

Kits can include probes for detection of alternative gene expression products in addition to antibodies for protein detection. The kit can optionally include a buffer. Reagents and standards can be provided in combinations reflecting the combinations of markers described herein as useful for detection.

### Devices

Devices are also provided. The devices can be used to carry out the methods described herein. Such devices can be adapted to receive assay materials that include surfaces to which are bound various probes for detection of gene expression products. In some embodiments, the device provides for an automated assay that provides a readout of the measured methylation of IBS biomarker genes, and generates the IBS methylation profiles, and, optionally, includes a processor that performs the classification of the generated IBS methylation profiles.

### EXAMPLE EMBODIMENTS

### Embodiment 1

A method of measuring DNA methylation in a biological sample obtained from a subject, the method comprising: (a) generating an IBS methylation profile from the biological sample obtained from the subject, wherein the profile comprises the presence of a plurality of IBS biomarker genes; and (b) measuring the amount of methylation in the IBS biomarker genes; wherein the amount of biomarker methylation is used to classify the profile.

### Embodiment 2

The method of embodiment 1, wherein the subject has manifested clinical symptoms associated with IBS.

### Embodiment 1

The method of a preceding embodiment, wherein the methylation profile is determined from a plurality of genes listed in Table 13 or Table 19.

### Embodiment 4

The method of a preceding embodiment, wherein generating the IBS methylation profile comprises preprocessing the biological sample with a kit for measuring the amount of methylation on all CpG sites.

### Embodiment 5

The method of a preceding embodiment, wherein the IBS biomarker genes are selected from differentially methylated genes between IBS and healthy controls and combinations thereof as shown in Table 3.

### Embodiment 6

The method of a preceding embodiment, wherein a computer algorithm determines a conditional probability of IBS based on the profile.

### Embodiment 7

The method of a preceding embodiment, wherein the biomarkers distinguish IBS from IBD.

### Embodiment 8

A method of screening for IBS in a subject, the method comprising: (a) generating an IBS methylation profile from a biological sample obtained from the subject, wherein the profile comprises the presence of a plurality of IBS biomarker genes; and (b) measuring the amount of methylation in the IBS biomarker genes; wherein the amount of biomarker methylation is used to classify the profile, and a subject is identified as having IBS based on the profile.

### Embodiment 9

A method of treating IBS comprising performing the measuring of claim 1 and administering treatment for IBS.

### Embodiment 10

The method of embodiment 9, wherein the treatment comprises administering rifaximin, loperamide, eluxadoline, alosetron, lubiprostone, linaclotide, plecanatide, a laxative, an antihistamine, an antispasmodic, a neuromodulator, dietary therapy, or behavioral therapy.

### Embodiment 11

The method of any preceding embodiment, wherein the biological sample comprises blood, plasma, serum, or mucosal tissue.

### Embodiment 12

The method of embodiment 11, wherein the sample is peripheral blood mononuclear cells (PBMCs), peripheral blood lymphocytes (PBL) or whole blood.

### Embodiment 13

The method of any preceding embodiment, wherein the amount of biomarker methylation is greater than 0.54.

### EXAMPLES

The following examples are presented to illustrate the present invention and to assist one of ordinary skill in making and using the same. The examples are not intended in any way to otherwise limit the scope of the invention.

### Example 1: DNA Methylation Profiling of Peripheral Blood Mononuclear Cells and Colonic Mucosa Identifies Biomarkers and Epigenetic Changes Associated with Irritable Bowel Syndrome

This Example demonstrates that DNA methylation of cell adhesion, ion transport and stress-related genes provide a link between EALs, peripheral mechanisms, and GI symptoms in IBS, and that a methylation-based PBMC profile is a promising diagnostic biomarker for IBS. DNA methylation of PBMCs and colonic mucosa in IBS (N=108, 102; 65-66% women) and HCs (N=36, 36; 53 and 56% women) was assessed using Illumina HM450 array. Gene expression was measured using QuantSeq RNA sequencing in mucosal biopsies. Differentially methylated positions and regions (DMPs and DMRs) were assessed in IBS and HCs along with epigenetic silencing of gene expression. Twelve and 7 DMRs (FDR<0.05) were associated with IBS vs HCs in PBMCs and colon, respectively. There were 179 and 231 DMPs (p<0.001) enriched for gene ontology terms including cell-adhesion and ion transport in PBMCs and colon. A signature of 550 DNA methylation-based biomarkers in PBMCs discriminated IBS from HCs with a 77% sensitivity and 91% specificity. DMPs in stress-related genes including glucocorticoid receptor, NR3C1 (PBMCs p=0.002, colon p=0.008) were associated with IBS, adverse childhood events (ACEs) (HDAC4 in PBMCs, FDR=0.04) and somatic symptom severity (FKBP4 in colon, FDR=0.03). Within IBS, we identified 3 methylation-based clusters in the colon. One cluster was associated with higher overall symptom and abdominal pain severity and was enriched in ion channel and neurotransmitter transport genes (FDR<0.05).

Irritable bowel syndrome (IBS) is a chronic gastrointestinal (GI) disorder characterized by abdominal pain associated with diarrhea and/or constipation¹. It has a high prevalence, affecting up to 11% of the population^{2,3}. Pathophysiology of IBS is not well understood, however, it is a heterogeneous disorder resulting from complex interactions between factors such as microbial dysbiosis within the gut, mucosal epithelial and immune function, visceral perception and central nervous system (CNS) modulation of gut signaling and motor function⁴.

Extensive preclinical and clinical evidence support the concept that IBS is a stress-related disorder. Chronic, sustained stressors experienced in childhood or adulthood have an increased prevalence in IBS and are associated with the onset and symptom flares⁵⁻⁷. Psychological stress can result in activation or dampened response of the hypothalamus-pituitary-adrenal (HPA) axis, autonomic nervous (ANS) and affect physiological functions of the GI tract⁵. However, the exact mechanisms of stress-related physiological changes in IBS is not well understood. It is known that chronic stress and other environmental factors, including early adverse life events (EALs) can trigger epigenetic changes, such as DNA methylation and histone modification, which have been implicated in the pathophysiology of several chronic diseases including cancer, chronic pain, and psychiatric diseases⁸⁻¹⁰. Epigenetic events, defined as changes in gene function that are not explained by changes in DNA sequence, can explain the variability observed in quantitative traits despite similarities in genetic background¹¹. Plasticity in neurobiological pathways regulating stress responsivity suggests a lifelong sensitivity to environmental cues, and epigenetic changes are shown to account for this plasticity¹².

DNA methylation, in particular, has emerged as a leading mechanism linking gene-environment interactions to long-term behavioral development, particularly in complex disorders^{13,14}. In normal mammalian somatic genomes, DNA methylation mainly occurs at Cytosines in a CpG dinucleotide context. CpG methylation is generally absent from short stretches of CpG-rich sequences known as CpG islands (CGIs) which typically occur at or near the transcription start site of genesis. Hypermethylation of CGI promoters is tightly linked with transcriptional repression of the affected gene and therefore have been viewed as an epimutation causing the silencing of a gene. In contrast, recent studies show that gene body methylation is positively correlated with gene expression and can be potential therapeutic targets¹⁶. However, the role of methylation is now thought to be much more complex, with a non-linear relationship between methylation and gene expression in many cases^{17,18}.

The effects of stress, largely mediated by the HPA axis, culminate in systemic secretion of glucocorticoids. Glucocorticoids activate glucocorticoid receptors (GR), which act as transcription factors regulating the expression of many downstream targets in a tissue specific manner. Methylation of the promoter region of NR3C1, the gene that codes for GR, in hippocampal tissue has been linked to an enhanced HPA axis response associated with early life stress in rats¹⁹. Lower expression of NR3C1 in the amygdala of female rats has been associated has been increased visceral hypersensitivity following early life stress compared to no stress controls²⁰. Moreover, chronic stress has been associated with an increase in epigenetic modification of genes that regulate visceral pain sensation in the peripheral nervous system of rats²¹. In humans, methylation of the promoter region 1F of the NR3C1 gene has been correlated with conditions such as maternal stress during pregnancy and childhood trauma²². In addition to NR3C1, other several other HPA axis and non HPA axis genes have been studied in stress-related conditions²³⁻²⁵, however, their methylation status in IBS remains to be explored. Additionally, stress (including defeat stress, seizures, posttraumatic) has been shown to induce long-lasting changes in the promoters of several genes, including brain derived neurotropic factor (BDNF)²⁶ and histone deacetylase 4 (HDAC4)²⁷, which in turn regulate the expression of several downstream genes. Interestingly, Sailaja et al²⁷ showed that daily administration of the histone deacetylase inhibitor sodium butyrate for 1 week after stress reversed the epigenetic changes (increased histone acetylation) and suppression of HDAC4 abolished the long-lasting acetylcholinesterase-related and behavioral stress effects, which may offer an additional avenue to treat stress-related diseases such as IBS.

Although a few diagnostic biomarkers have been proposed in IBS, they perform modestly in predicting IBS²⁸. DNA methylation marks have been proposed as diagnostic biomarkers in cancer²⁹⁻³¹, however, they have not been explored in IBS. Our earlier pilot study on genome-wide methylation in peripheral blood mononuclear cells (PBMCs) identified neuronal and oxidative stress related genes to be associated with IBS compared to healthy controls (HCs).³² There is a lack of studies exploring epigenetic changes associated with colonic mucosa in IBS. Nonetheless, epigenetic marks can potentially serve as diagnostic biomarkers and also lend insight into the pathophysiological mechanisms of IBS.

Therefore, the study sought to: 1) Compare genome-wide DNA methylation in PBMCs and colonic mucosa of IBS patients compared to HCs, including investigation of stress-related genes, 2) Investigate potential methylation-based biomarkers, subtype analysis and clinical associations, 3) Identify gene expression differences in colonic mucosa associated with epigenetically silenced genes in IBS and HCs, and 4) Identify common IBS associated epigenetic changes in PBMCs and colon.

### Methods

### Study Subjects and Recruitment

Male and female IBS patients and healthy controls who were 18-55 years of age were predominantly recruited from community advertisements. A medical history and physical examination was performed in all participants. The diagnosis of IBS and bowel habit subtypes (IBS with diarrhea [IBS-D], constipation [IBS-C] and mixed pattern [IBS-M]) were based on the Rome II diagnostic criteria¹ and were confirmed by a gastroenterologist with expertise in IBS (LC). The patients had no evidence of organic gastrointestinal disease. HCs had no personal or family history of IBS or other chronic pain conditions. Medication history was collected in all subjects. Additional exclusion criteria for all subjects included a history of chronic infectious or inflammatory disorders, active psychiatric illness over the past 6 months as assessed by structured clinical interview for the DSM-IV (MINI)³, smoking more than 0.5 packages of cigarettes daily, daily intake of >400 mg caffeine (equivalent to a 16 oz cup of standard-brew coffee), or exercise 1 hour or more per day. Subjects were compensated for participating in the study. The study was approved by our Institutional Review Board (IRB). Informed consent was obtained from all subjects.

### Symptom Measures

At the screening visit, a bowel symptom questionnaire was used to assess the presence and severity of IBS symptoms and duration of disease³⁴. It included the Rome III diagnostic questions for IBS, bowel habit subtypes, demographic characteristics, current abdominal pain severity (0-20), usual IBS severity score (“How bad are your symptoms usually?” None [0] to very severe [5]) and a second disease severity measurement tool, IBS symptom severity score (IBS-SSS). This scale evaluates primarily the intensity of IBS symptoms during a 10-day period: abdominal pain distension, stool frequency and consistency, and interference with life in general (0-500). Validated questionnaires were administered to patients and HCs to assess psychological and somatic symptoms. The Hospital Anxiety and Depression Scale (HAD) is a widely used 14-item questionnaire for assessing current symptoms of anxiety and depression³⁵. The presence of EALs before age 18 was measured using the ACE (adverse childhood experiences) questionnaire, with 18 questions in 8 domains⁷ and the ACE score is calculated by assigning 1 point for each domain (“Yes”=1 or “No”=0; ACE score range of 0-8) of physical (1), emotional (2), and sexual abuse (4), and household substance abuse (2), parental separation or divorce (1), mental illness in household (2), incarcerated household member (1), and parent treated violently (2). The Perceived Stress Scale (PSS)³⁶ is a validated 10-item questionnaire used to evaluate the association of perceived stress over the past 1-month with disease severity in chronic conditions. Blood samples were collected at the screening visit or at the flexible sigmoidoscopy procedure.

### Collection of Colonic Mucosal Tissue

A flexible sigmoidoscopy to at least 40 cm from the anal verge was performed. Subjects were instructed to use two tap-water enemas as the bowel preparation. During the sigmoidoscopy, colon biopsies were taken at 30 cm from the anal verge. The tissues were snap frozen in liquid Nitrogen or stored in RNALater™, according to manufacturer's instructions. Two biopsies per subject were used for the study, one snap frozen biopsy for DNA methylation and one RNA later biopsy for gene expression analysis.

### DNA and RNA Extraction

PBMCs were isolated from whole blood of study participants collected in anti-coagulant (EDTA) tubes, using Ficoll-Paque method. DNA from PBMCs and colon biopsies was extracted using DNeasy Blood & Tissue Kit, Qiagen Inc., USA. RNA was extracted using RNeasy Plus Mini kit, Qiagen Inc., with genomic DNA eliminator column from Qiagen™. Quantities of DNA and RNA were measured using PicoGreen™ and RiboGreen™ fluorescent assays, ThermoFisher Scientific. RNA purity and integrity was measured using the Agilent 2100 bioanalyzer (Agilent Technologies, USA).

### DNA Methylation Array

For global methylation profiling, we used the Illumina Infinium HumanMethylation450 (HM450) BeadChip (Illumina, San Diego, Calif.), which interrogates DNA methylation status of >450,000 CpGs and >99% of all genes. We performed bisulfite conversion on 1 μg of genomic DNA from each sample using the EZ-96 DNA Methylation Kit (Zymo Research, Irvine, Calif.) according to the manufacturer's instructions and as described previously³² and hybridized to HM450 BeadChips. These were subsequently scanned using the Illumina HiScanSQ system. Raw intensity data were exported from Illumina GenomeStudio (version 2011.1).

### Gene Expression

Gene expression was measured using QuantSeq 3′ mRNA sequencing³⁷. QuantSeq library preparation: Normalized quantities of RNA were converted into cDNA by using QuantSeq 3′mRNA-Seq Reverse (REV) Library Prep Kit (Lexogen) according to manufacturer's instruction to generate compatible library for Illumina sequencing. cDNA libraries were assessed using TapeStation (Agilent Technologies, USA) before 100 bp single end sequencing using Illumina HiSeq 2500 system at UCLA Neuroscience Genomics Core, based on standard protocols.

### Selection of Stress Genes

Using the search terms in PubMed, ‘psychosocial stress AND stress-response genes’, 28 abstracts were downloaded and a gene list was manually curated from the full text. Additional papers were referred to wherever necessary. Enrichment of a gene set in a list compared to the global background was performed using hypergeometric test³⁸ in ‘stats’ package and Fisher's Exact test.

### Statistical Methods and Bioinformatic Analyses

DNA Methylation

Data was normalized using functional normalization³⁹, in order to preserve large tissue-related differences. Of the 485,577 CpG probes on the array, we filtered out probes with high detection p values (n=13326, p<0.01), cross reactive probes (probes with probes with at least 50 nucleotide homology [29], n=26058), probes with a SNP and repeat regions within 10 base pairs of the target CpG [30], n=15168) and probes on X and Y chromosomes (n=10703), leaving 420257 probes for analysis. Bata values were converted to M-values before running differential methylation analysis. Batch effects were visualized using hierarchical clustering of 1000 most variant methylation probes. Since we did not observe clustering of batches, no batch effect correction was applied. Cellular abundances were estimated in the PBMCs and compared between IBS and HCs using ‘The epigenetic clock’ software⁴⁰ which uses method and R code described by Houseman⁴¹. Since the distribution of various cell types was not significantly different between IBS and HCs, except CD8T (slightly elevated in IBS, mean IBS=0.077%, mean HCs=0.046%, p=0.02), no further correction was applied. Differentially methylated positions (DMPs) and differentially methylated regions (DMRs) were tested using “minfi”⁴² and “DMRcate”⁴³ respectively. Biomarker discovery was performed using random forest classification⁴⁴. A FDR corrected p value <0.05 (equivalent to p<1.0E-07) was considered statistically significant. For suggestive DMPs, an arbitrary threshold of p<5.0E-05 was used. We used a cutoff of p<0.001 to identify DMRs and for gene ontology (GO) analysis. Additional analyses have been detailed in the Supplementary materials.

Biomarker Discovery

Random Forest classification⁴⁵ was used to train the data to identify a set of biomarkers capable of discriminating IBS against HCs. We selected a combination of markers with minimum classification error rate using wrapper method as described in https://www.analyticsvidhya.com/blog/2016/12/introduction-to-feature-selection-methods-with-an-example-or-how-to-select-the-right-variables/ (published December 2016 at the site analyticsvidhya-dot-com as “Introduction to Feature Selection Methods With An Example or How to Select the Right Variables”. Briefly, 109 IBS and 36 HCs were divided into a balanced set of 40 IBS and 36 HCs. These 76 samples were divided into training and test datasets (⅔^{rd} and ⅓^{rd}, respectively). Starting from a set of probes/features that were associated with IBS (P<0.05), we sorted the features based on the variable importance scores for the training data set and tested the accuracy and error rate of the classification using 500 tress, by adding 50 most important features, incrementally. For the selected set of probes with least error rate, we calculated the Area under the Curve (AUC), positive and negative predictive values (PPV and NPV, respectively) using pROC package⁴⁶, and selected cutoffs based on Youden's index⁴⁷ and to maximize sensitivity without significantly compromising specificity. We then tested this on another test cohort comprising of non-overlapping IBS samples.

IBS Subtype Analysis

The presence of methylation-based subtypes within IBS was tested using consensus clustering in ‘ConsensusClusterPlus’ package⁴⁸, using algorithms such as ‘pam’, ‘heirarchical’, dianaHook and ‘kmeans’ on the 5000 most variant probes. The association of clusters with clinical features was tested using linear regression controlling for age and sex variables. Since BMI correlated with age, it was not included in the model.

Gene Expression Analysis

Count data were extracted from Lexogen QuantSeq data analysis pipeline, “bluebee” (https://www.bluebee.com/lexogen/). Differentially expressed genes were identified using “DESeq2”⁴⁹ after controlling for batch effects (lanes).

Gene Ontology (GO) Term Enrichment Analysis

Functional annotation was performed using GO term enrichment analysis to highlight the most relevant GO terms associated with a given gene list, using The Database for Annotation, Visualization and Integrated Discovery (DAVID) bioinformatics resources tool. v6.8⁵⁰.

### Results

Table 1 shows the clinical characteristic of the study population. Age, body mass index (BMI) and proportion of women were not significantly different between IBS (N=109) and HCs (N=36). PBMCs were extracted from 108 IBS patients (36 IBS-C, 36 IBS-D and 36 IBS-M) and 36 HCs. One hundred and two IBS patients (36 IBS-C, 35 IBS-D and 31 IBS-M) and 36 HCs underwent sigmoidoscopy with sigmoid colon biopsies. There was a 77% overlap (n=106) between subjects that contributed PBMCs (n=145) and colonic mucosal biopsies (n=138). Gene expression and methylation data were available on 97% of colonic mucosal biopsies. A small number of patients were taking anxiolytics (n=8) or antidepressants (n=4).

This Table shows clinical characteristics of IBS patients and healthy controls used for DNA methylation analysis in PBMCs and colon biopsies. Range of scores are mentioned in the parentheses.

| | PBMCs | | | | Colon | | | |
|---|---|---|---|---|---|---|---|---|
| | | | Healthy | | | | Healthy | |
| | | | Controls | | | | Controls | |
| Clinical characteristic | IBS (N = 109) | | (N = 36) | | IBS (N = 102) | | (N = 36) | |
| Age [mean(SD)] | 30.88 | (10.83) | 32.97 | (9.38) | 33.14 | (11.46) | 33.69 | (7.69) |
| BMI Age [mean(SD)] | 25.1 | (4.77) | 26.67 | (4.24) | 25.27 | (4.70) | 26.54 | (4.17) |
| Female % | 65 | | 52.78 | | 65.69 | | 55.56 | |
| Bowel Habits (IBS-C, | 33.03, | | — | | 35.29, | | — | |
| IBS-D, | 33.03, | | | | 34.31, | | | |
| IBS-M)% | 33.94 | | | | 30.39 | | | |
| Overall Severity [mean(SD)] (0-20) | 9.52 | (4.32) | — | | 9.54 | (4.34) | — | |
| Abdominal Pain [mean(SD)] (0-20) | 9.11 | (4.28) | — | | 9.37 | (4.12) | — | |
| Bloating [mean(SD)] (0-20) | 11.06 | (4.77) | 0.82 | (1.51) | 11.34 | (4.79) | — | |
| Usual Severity [mean(SD)] (1-5) | 3.21 | (0.66) | 1.2 | (0.45) | 3.18 | (0.66) | — | |
| ACE Score [mean(SD)] (0-8) | 1.91 | (1.79) | 1.46 | (1.77) | 2.06 | (1.88) | 1.56 | (1-8) |
| VSI Score [mean(SD)] (0-75) | 37.21 | (15.69) | 3.66 | (6.92) | 36.17 | (15.71) | 2.83 | (6.46) |
| PSS Score [mean(SD)] (0-40) | 16.14 | (8.03) | 9.83 | (7.06) | 15.34 | (8.17) | 8.8 | (6.28) |
| PHQ-15 Score [mean(SD)] (0-30) | 10.57 | (4.57) | 1.59 | (1.83) | 10.62 | (4.93) | 1.37 | (1.44) |
| IBS-SSS [mean(SD)] (0-500) | 228.04 | (93.74) | — | | 232.66 | (90.56) | — | |
| HAD Anxiety [mean(SD)] (0-21) | 7.33 | (4.28) | 3.09 | (2.91) | 7.01 | (4.59) | 2.72 | (2.63) |
| HAD Depression [mean(SD)] (0-21) | 3.39 | (3.21) | 1.86 | (2.45) | 3.45 | (3.55) | 1.28 | (2.13) |

Abbreviations: BMI, body mass index; IBS-C, IBS constipation subtype, IBS-D, diarrhea subtype, IBS-M, mixed subtype; ACE, adverse childhood events; VSI, visceral sensitivity index; PSS, perceived stress score; PHQ-15, patient health questionnaire, somatization score; IBS-SSS, IBS symptom severity score; HAD, hospital anxiety depression scale.

### DNA Methylation Profile of IBS Patients Compared to HCs in PBMCs

Methylation Differences Between IBS Vs HCs

We found 7DMPs (p<5.0E-05) that were hypermethylated in IBS compared to HCs; however, no significant DMPs were found at FDR<0.05. GO functional annotation of genes harboring 179 CpGs with a p:0.001, revealed ‘cell adhesion’ as one of the top terms associated with the list (Table 2). Protocadherin 17 (PCDH17), implicated in cell adhesion was one of the top differentially methylated genes. We found 12 promoter associated DMRs (FDR<0.05) to behypermethylated in IBS compared to HCs. We found DMRs in Guanine nucleotide-binding protein G(s) subunit alpha (GNAS), with 5consecutive CpGs, and PCDH17, with 3CpGs, genes hypermethylated in IBS compared to healthy controls. As shown in Table 3, 5 of the 12DMRs in PBMCs were glycoproteins, associated with functions such as ‘cell adhesion’ (cell-cell connections in brain), ‘calcium signaling’ and ‘oxidation-reduction’ among others.

TABLE 2
Gene ontology terms associated with differentially methylated
positions (DMAs) in IBS compared to HCs in PBMCs and colon.

| | | | Methylation | | |
|---|---|---|---|---|---|
| | | | status | Enrichment | |
| GO term | Count | Genes | (hyper, hypo) | p | FDR |
| PBMCs | | | | | |
| Cell-cell adherens | 23 | ATIC, ERC1, EPS8L1, GIPC1, | 20, 3 | 2.70E−04 | 0.380 |
| junction | | MKL2, CADM3, CLIC1, | | | |
| | | CKAP5, DLG1, FLOT2, | | | |
| | | FNBP1L, HSPA1B, ITGB1, | | | |
| | | PCBP1, RTN4, RSL1D1, | | | |
| | | STK24, SNX2, SPTBN2, | | | |
| | | SND1, STX5, TES, ZC3HAV1 | | | |
| Pleckstrin homology- | 28 | BBSS, CDC42BPA, DAB1, | 24, 4 | 2.10E−04 | 0.340 |
| like domain | | EPS8L1, EPS8L3, MCF2L, | | | |
| | | MCF2L2, RASA3, ARHGEF17, | | | |
| | | SH2B1, TBC1D2B, WBP2, | | | |
| | | APBA1, APBA2, ANKS1B, | | | |
| | | DGKD, ELMO1, EPB41, | | | |
| | | FAM43B, MAPK8IP2, PLCD3, | | | |
| | | PLEKHG1, SPTBN2, SPTBN4, | | | |
| | | SPATA13, SKAP2, TECPR1, | | | |
| | | VAV2 | | | |
| Thrombospondin, | 12 | ADAMTS19, ADAMTS2, | 12, 0 | 3.10E−06 | 0.005 |
| type 1 repeat | | ADAMTS5, ADAMTS8, | | | |
| | | ADAMTSL3, RSPO4, SSPO, | | | |
| | | PAPLN, SEMA5A, SPON2, | | | |
| | | THBS1, UNC5A | | | |
| Colon | | | | | |
| mRNA transport | 5 | SUPT6H, THOC3, THOC7, | 4, 1 | 7.80E−03 | 9.500 |
| | | LRPPRC, NUP205 | | | |
| Cul3-RING ubiquitin | 4 | KLHL14, KLHL36, KBTBD11, | 2, 2 | 1.70E−02 | 19.00 |
| ligase complex | | KCTD2 | | | |
| Lysosome | 5 | NPC1, GALC, HGSNAT, | 4, 1 | 3.00E−02 | 35.00 |
| | | LITAF, MAN2B1 | | | |
| ion transport domain | 4 | ITPR2, KCNMA1, KCNG3, | 3, 1 | 4.80E−02 | 49.00 |
| | | KCNQ3 | | | |

Table 3: This Table shows differentially methylated regions (DMRs) between IBS and healthy controls in the promoter of genes listed, along with the number of CpG sites, mean beta fold change between IBS and HCs in PBMCs as well as colon, false discovery rate (FDR) calculated as adjusted p-value from the CpGs constituting the significant region, and gene ontology (GO) functional term.

| | | | CpGs | | | |
|---|---|---|---|---|---|---|
| Gene | Name | Chr | (n) | FC | FDR | GO Functional Term |
| PBMCs | | | | | | |
| HEXDC | Hexosaminidase | chr17 | 5 | 0.013 | 1.89E−06 | carbohydrate metabolic |
| | | | | | | process |
| RP11 | Retinitis pigmentosa | chr1 | 5 | 0.035 | 8.55E−06 | mRNA splicing |
| | 11 | | | | | |
| PCDH17 | Protocadherin 17 | chr13 | 3 | 0.012 | 1.40E−05 | Glycoprotien, cell adhesion |
| MATN4 | Matrilin 4 | chr20 | 9 | 0.025 | 2.33E−05 | Glycoprotein, extracellular |
| | | | | | | matrix |
| GNAS | Guanine nucleotide- | chr20 | 18 | 0.018 | 8.79E−05 | Glycoprotein, cAMP |
| | binding protein G(s) | | | | | dependent Calcium signaling |
| | subunit alpha | | | | | |
| DECR2 | 2,4-dienoyl CoA | chr16 | 5 | 0.055 | 8.79E−05 | oxidation reduction |
| | reductase 2 | | | | | |
| DNAI2 | Dynein Axonemal | chr17 | 3 | 0.029 | 0.0002 | Microtubule motor activity |
| | Intermediate Chain 2 | | | | | |
| SIGLEC15 | Sialic acid binding Ig | chr18 | 2 | 0.040 | 0.0006 | Glycoprotein, function not |
| | like lectin 15 | | | | | known |
| CYP1A1 | Cytochrome P450 | chr15 | 2 | 0.016 | 0.0008 | Glycoprotein, oxidation |
| | family 1 subfamily A | | | | | reduction |
| | member 1 | | | | | |
| USP29 | Ubiquitin specific | chr19 | 8 | 0.018 | 0.0008 | peptidase |
| | peptidase 29 | | | | | |
| Unannotated | Unannotated | chr6 | 3 | 0.014 | 0.0003 | Unannotated |
| Unannotated | Unannotated | chr16 | 4 | 0.028 | 0.0008 | Unannotated |
| Colon | | | | | | |
| DGUOK | Deoxyguanosine | chr2 | 8 | −0.017 | 2.16E−11 | negative regulation of neuron |
| | kinase | | | | | projection development |
| STPG2 | Sperm-tail PG-rich | chr4 | 9 | −0.015 | 4.85E−07 | NA |
| | repeat containing 2 | | | | | |
| CAT | Catalase | chr11 | 13 | 0.02 | 6.65E−06 | Antioxidant activity |
| GALC | Galactosylceramidase | chr14 | 8 | 0.017 | 0.000101 | carbohydrate metabolic |
| | | | | | | process |
| SLC38A4 | Solute carrier family | chr12 | 12 | 3.02 | 0.000123 | Ion transport |
| | 38, member 4 | | | | | |
| STC2 | Stanniocalcin 2 | chr5 | 3 | 4.40 | 0.000146 | Response to oxidative stress; |
| | | | | | | calcium ion homeostasis |
| COX4I2 | Cytochrome c oxidase | chr20 | 8 | 2.80 | 0.000256 | Oxidation-reduction |
| | subunit IV isoform 2 | | | | | |

GO, gene ontology; chr, chromosome; FC, fold change; FDR, false detection rate.

Methylation of Stress-Related Genes in IBS Compared to HCs

The PubMed IDs for all the stress-related genes investigated are shown in U.S. provisional patent application No. 62/678,618, filed May 31, 2018. Of the 2108 CpG sites in 86 unique stress-related genes, 88 CpG sites were associated with IBS (p<0.05), which indicated a potential enrichment of stress-related genes in IBS-associated DMPs (Hypergeometric test (dhyper) p=0.002, Fisher test p=0.02, OR=1.29, 95% CI=1.03-1.59). Seventy of the 88 CpGs were hypermethylated in IBS patients compared to HCs. These included a CpG site in the 1F promoter of NR3C1 gene and five in the promoter of HDAC4 gene. Compared to HCs, IBS also had two hypermethylated CpG sites (cg06640763, p=0.005 and cg12243858, p=0.031) in the SCO-spondin (SSPO) gene, which was associated with IBS in our previous study³².

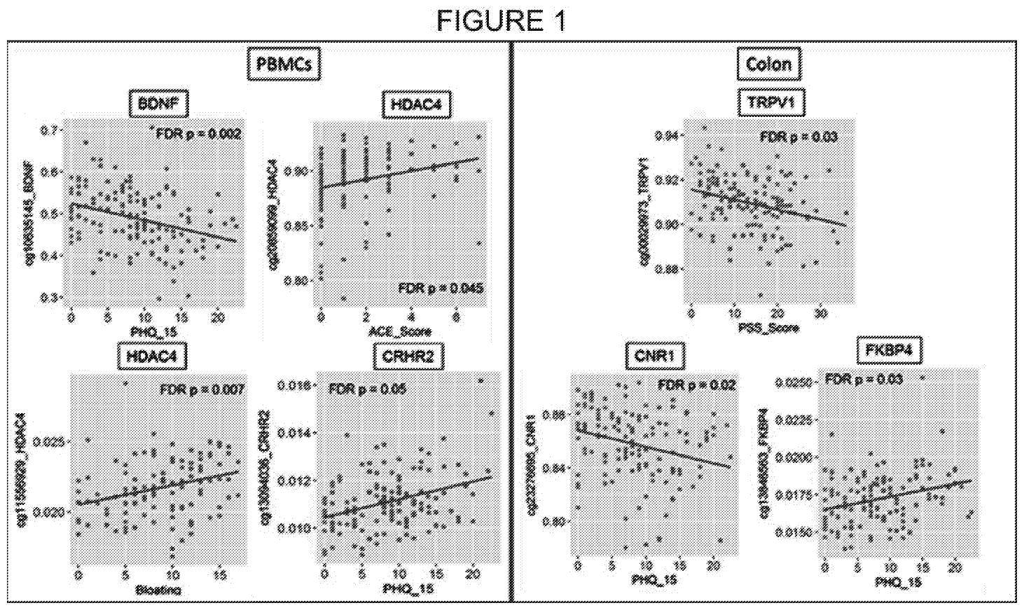

We found significant correlations between methylation of 5 CpG sites in 3 stress-related genes and clinical variables (FDR<0.05, FIG. 1. Hypermethylation of a CpG site in the BDNF gene, which plays an important role in synaptic plasticity, was associated with lower PHQ-15 (somatic symptom severity) scores. In contrast, hypermethylation of corticotropin-releasing factor receptor 2 (CRHR2), a principal neuroregulator of the HPA axis, was associated with higher PHQ-15 scores. Hypermethylation of the HDAC4 gene was associated with higher abdominal pain, bloating and ACE scores.

DNA Methylation-Based Biomarker for IBS

Of the 13,698 IBS associated CpG sites (p<0.05), we selected 550 features that discriminated IBS from HCs. The overall classification error for the training set (out-of-bag (OOB) estimate of error rate) was 9.8%. Once the classifier was trained and tested (cross-validation) on one set of IBS and HCs, we cross-validated the classifier in a second group of IBS patients. FIG. 2 shows the ROC curve to assess the performance of the biomarkers and average scores (methylation beta values for those markers) that were obtained from the cross-validation data. The area under the ROC curve (AUC) was 0.92 for the IBS vs HC group. A cutoff of 0.55, gave a maximum sensitivity 77% for the highest specificity 91% with a PPV of 91% and a NPV of 79% (Table 4).

TABLE 4
Random forest classification error rates
for training and test data sets.

| | Cutoff 1 | Cutoff 2 | Cutoff 3 | Cutoff 4 |
|---|---|---|---|---|
| Threshold | 0.45 | 0.502 | 0.552 | 0.565 |
| Sensitivity | 100 | 84.61 | 76.92 | 69.23 |
| Specificity | 66.67 | 75 | 31.67 | 100 |
| PPV | 76.47 | 78.57 | 90.91 | 100 |
| NPV | 100 | 81.82 | 78.57 | 75 |

Legend: This Table shows sensitivity, specificity, negative predictive value (NPV) and positive predictive value (PPV) for various cutoffs (threshold, local maximas). A cutoff of 0.552 gave a maximum Sensitivity at a high Specificity as was chosen as an optimal cutoff.

Cross-validation in the second data set comprising of IBS patients only resulted in a 16% misclassification with 11/69 IBS patients being misclassified HCs (Table 5). This rate was similar to that observed in the first test data set Seven out of the 11 misclassified IBS patients were men (Fisher's exact p=0.08). The misclassified group was also associated with numerically lower mean bloating scores compared to the correctly classified group (9 vs 11, p=0.16). 271 genes associated with 417 hypermethylated CpGs out of the 550 markers were enriched in GO categories including ‘cell-membrane’ and ‘bicellular tight junction assembly’ and included several genes associated with ion transport, such as KCNJ9, SLC9A2, CACNA1H, KCNQ1, TRPM2 and SLC9A1133. Genes associated with hypomethylated CpGs were associated with GO terms such as, ‘zinc finger’, and marginally associated with the term ‘Rho guanyl-nucleotide exchange factor activity’. Supplementary Table 1.4 of U.S. provisional patent application No. 62/678,618, filed May 31, 2018, lists of all CpG sites that were used in the biomarker discovery panel.

TABLE 5
Random forest classification misclassification
rates for training and test data sets.

| | Total N (IBS, HC) | Misclassification | |
|---|---|---|---|
| Training | 51 (27, 24) | 5/51 | (9.8%) |
| Test 1 | 25 (13, 12) | 5/25 | (20%) |
| Test 2 (IBS | 69 | 11/69 | (16%) |
| only) | | | |

Legend: Table 5 shows classification error rates for all the three data sets used for the analysis. The third data set consisted on IBS subjects only. HC, healthy controls.

Methylation Differences Between IBS Bowel Habit Subtypes

There were no DMPs with FDR<0.05 between bowel habit subtypes. However, we found significant DMRs between bowel habit subtypes and HCs which are listed in Table 6. Twenty-one of the 24 DMRs between IBS-C vs HCs were hypermethylated in IBS-C and included genes such as Homeobox protein HOXA5, which is a transcription factor, and plays a role during embryonic development, and GNAS, associated with cyclic adenosine monophosphate (cAMP) mediated signaling. The 12 DMRs associated with IBS-D compared to HCs included genes such as RNF135, which is associated with innate immune defense against viruses⁵¹, among others. IBS-M was associated with 59 DMRs (55/59 hyper methylated) which included PCDH17, associated with cell adhesion and CYP1A1 associated with drug metabolism. Compared to IBS-D, IBS-C was associated with a hypomethylation of genes including HOXA5 and HOXA6. A promoter associated DMR in Nuclear Receptor Subfamily 4Group A Member 2 (NR4A2) gene, which is involved in generation and maintenance of dopaminergic neurons⁵², was hypomethylated in IBS-C compared to IBS-D and HCs (FDR <0.05).

TABLE 6
IBS bowel habit subtype associated differentially methylated regions in PBMCs.

| | | | | | | | Gene |
|---|---|---|---|---|---|---|---|
| Chromosome | Start | End | Width | No. cpgs | FDR | MeanBetaFC | Symbol |
| IBS-D vs HC | | | | | | | |
| chr17 | 29297391 | 29297873 | 483 | 13 | 1.32E−05 | 0.031 | RNF135 |
| chr12 | 47219626 | 47220092 | 467 | 12 | 1.36E−08 | 0.053 | SLC38A4 |
| chr15 | 74218418 | 74218921 | 504 | 11 | 1.83E−04 | 0.022 | LOXL1 |
| chr13 | 31506685 | 31507139 | 455 | 8 | 8.18E−05 | −0.036 | TEX26 |
| chr19 | 9785295 | 9785906 | 612 | 7 | 2.42E−04 | 0.036 | ZNF562 |
| chr20 | 25039210 | 25039812 | 603 | 7 | 1.32E−05 | 0.021 | ACSS1 |
| chr11 | 1.22E+08 | 1.22E+08 | 484 | 6 | 2.24E−04 | −0.025 | MIR125B1 |
| chr17 | 72270028 | 72270444 | 417 | 5 | 1.61E−04 | 0.026 | DNAI2 |
| chr1 | 3105151 | 3105326 | 176 | 4 | 2.38E−04 | 0.086 | NA |
| chr2 | 7171869 | 7172097 | 229 | 4 | 9.32E−05 | 0.030 | NA |
| chr4 | 44727930 | 44728051 | 122 | 3 | 2.32E−04 | 0.005 | GNPDA2 |
| chr16 | 450837 | 450970 | 134 | 3 | 7.00E−04 | 0.062 | DECR2 |
| IBS-C vs HC | | | | | | | |
| chr7 | 27183133 | 27185282 | 2150 | 47 | 2.13E−09 | −0.020 | HOXA5 |
| chr20 | 57427017 | 57427973 | 957 | 29 | 5.99E−07 | 0.019 | GNAS |
| chr6 | 31539539 | 31540461 | 923 | 19 | 5.51E−06 | 0.037 | LTA |
| chr19 | 57630034 | 57630742 | 709 | 16 | 7.62E−06 | 0.021 | USP29 |
| chr14 | 24640947 | 24642317 | 1371 | 14 | 6.69E−09 | −0.024 | REC8 |
| chr17 | 41277974 | 41278712 | 739 | 14 | 5.72E−06 | 0.034 | BRCA1 |
| chr22 | 22901267 | 22902237 | 971 | 12 | 3.32E−05 | 0.018 | PRAME |
| chr19 | 51219975 | 51220537 | 563 | 10 | 2.72E−06 | 0.014 | SHANK1 |
| chr15 | 91473059 | 91473569 | 511 | 9 | 3.55E−06 | 0.039 | UNC45A |
| chr20 | 43935222 | 43935551 | 330 | 9 | 3.24E−06 | 0.032 | MATN4 |
| chr5 | 23506738 | 23507656 | 919 | 9 | 2.13E−09 | 0.044 | PRDM9 |
| chr3 | 1.39E+08 | 1.39E+08 | 273 | 7 | 3.36E−04 | 0.020 | PRR23A |
| chr5 | 8457548 | 8458392 | 845 | 7 | 2.52E−08 | 0.051 | RP11 |
| chr12 | 1.31E+08 | 1.31E+08 | 566 | 5 | 2.67E−04 | 0.047 | PIWIL1 |
| chr15 | 40571997 | 40572794 | 798 | 5 | 2.12E−05 | 0.020 | ANKRD63 |
| chr17 | 80393124 | 80393666 | 543 | 5 | 2.13E−09 | 0.017 | HEXDC |
| chr19 | 55660514 | 55660625 | 112 | 5 | 4.22E−04 | 0.072 | TNNT1 |
| chr2 | 1.19E+08 | 1.19E+08 | 422 | 5 | 2.17E−04 | 0.056 | HTR5BP |
| chr2 | 1.57E+08 | 1.57E+08 | 426 | 5 | 8.31E−05 | −0.028 | NR4A2 |
| chr8 | 1.45E+08 | 1.45E+08 | 340 | 5 | 6.52E−04 | 0.034 | ZNF707 |
| chr10 | 3824387 | 3824687 | 301 | 4 | 1.40E−04 | 0.042 | KLF6 |
| chr16 | 302852 | 303192 | 341 | 4 | 8.26E−05 | 0.041 | ITFG3 |
| chr15 | 75019283 | 75019376 | 94 | 3 | 5.57E−04 | 0.028 | CYP1A1 |
| chr5 | 42924215 | 42924552 | 338 | 3 | 1.46E−05 | 0.056 | NA |
| IBS-M vs HC | | | | | | | |
| chr20 | 36148133 | 36150061 | 1929 | 44 | 1.18E−09 | 0.015 | NNAT |
| chr6 | 31126599 | 31127863 | 1265 | 23 | 8.95E−09 | −0.015 | TCF19 |
| chr11 | 7041194 | 7041987 | 794 | 18 | 1.32E−05 | 0.013 | ZNF214 |
| chr6 | 29944771 | 29945728 | 958 | 17 | 1.19E−04 | 0.011 | HCG9 |
| chr5 | 5139069 | 5140029 | 961 | 16 | 6.19E−05 | 0.020 | ADAMTS16 |
| chr22 | 45809244 | 45809952 | 709 | 15 | 1.41E−04 | 0.024 | RIBC2 |
| chr3 | 24536309 | 24537277 | 969 | 15 | 5.78E−05 | 0.015 | THRB |
| chr15 | 74218418 | 74219307 | 890 | 12 | 3.16E−07 | 0.022 | LOXL1 |
| chr17 | 6899085 | 6899577 | 493 | 11 | 4.17E−04 | 0.034 | ALOX12 |
| chr15 | 75018852 | 75019376 | 525 | 11 | 1.26E−07 | 0.038 | CYP1A1 |
| chr6 | 32063991 | 32064258 | 268 | 11 | 4.37E−04 | 0.047 | NA |
| chr10 | 60936232 | 60937048 | 817 | 11 | 2.56E−04 | 0.016 | PHYHIPL |
| chr6 | 84418724 | 84419360 | 637 | 11 | 5.73E−04 | 0.012 | SNAP91 |
| chr19 | 57630446 | 57630742 | 297 | 10 | 2.95E−04 | 0.019 | USP29 |
| chr2 | 74875227 | 74875932 | 706 | 9 | 1.71E−05 | 0.028 | M1AP |
| chr6 | 29943188 | 29943480 | 293 | 9 | 4.53E−04 | 0.011 | HCG9 |
| chr20 | 43935222 | 43935551 | 330 | 9 | 4.45E−08 | 0.031 | MATN4 |
| chr7 | 1.23E+08 | 1.23E+08 | 369 | 8 | 3.86E−04 | 0.010 | CADPS2 |
| chr20 | 25128681 | 25129184 | 504 | 8 | 4.43E−04 | 0.020 | RP4 |
| chr4 | 1107202 | 1107983 | 782 | 8 | 5.24E−06 | 0.032 | RNF212 |
| chr22 | 19949585 | 19950166 | 582 | 8 | 4.52E−06 | −0.028 | COMT |
| chr20 | 982623 | 983130 | 508 | 8 | 4.09E−04 | 0.012 | RSPO4 |
| chr5 | 8457548 | 8458392 | 845 | 7 | 5.04E−08 | 0.047 | RP11 |
| chr19 | 36246395 | 36246906 | 512 | 7 | 1.92E−05 | 0.045 | HSPB6 |
| chr3 | 1.39E+08 | 1.39E+08 | 273 | 7 | 8.65E−04 | 0.021 | PRR23A |
| chr8 | 52321814 | 52322341 | 528 | 7 | 1.16E−05 | 0.026 | PXDNL |
| chr14 | 1.06E+08 | 1.06E+08 | 879 | 7 | 1.92E−05 | 0.016 | IGHM |
| chr16 | 51184152 | 51184583 | 432 | 7 | 1.95E−04 | 0.014 | SALL1 |
| chr1 | 24229232 | 24230384 | 1153 | 6 | 1.18E−09 | 0.040 | RP11 |
| chr2 | 74729279 | 74729710 | 432 | 6 | 4.78E−04 | 0.031 | LBX2 |
| chr11 | 15095017 | 15095178 | 162 | 6 | 1.25E−04 | 0.020 | CALCB |
| chr3 | 1.39E+08 | 1.39E+08 | 301 | 6 | 1.29E−04 | 0.035 | PRR23B |
| chr4 | 57547347 | 57547999 | 653 | 6 | 7.20E−05 | −0.046 | HOPX |
| chr4 | 1.41E+08 | 1.41E+08 | 332 | 6 | 7.05E−04 | 0.016 | UCP1 |
| chr10 | 1.32E+08 | 1.32E+08 | 353 | 5 | 7.14E−04 | 0.032 | NA |
| chr16 | 3079423 | 3079953 | 531 | 5 | 2.24E−05 | 0.043 | NA |
| chr5 | 1.71E+08 | 1.71E+08 | 268 | 5 | 4.17E−04 | 0.053 | NA |
| chr17 | 80393124 | 80393666 | 543 | 5 | 5.80E−06 | 0.014 | HEXDC |
| chr13 | 58204365 | 58205111 | 747 | 5 | 1.26E−05 | 0.015 | PCDH17 |
| chr7 | 56297490 | 56297714 | 225 | 5 | 4.62E−04 | 0.047 | CCNJP1 |
| chr1 | 2.48E+08 | 2.48E+08 | 181 | 5 | 8.51E−04 | 0.019 | TRIM58 |
| chr19 | 49828641 | 49828833 | 193 | 5 | 9.41E−04 | 0.016 | SLC6A16 |
| chr10 | 57389779 | 57390306 | 528 | 4 | 2.67E−04 | 0.016 | NA |
| chr11 | 14280742 | 14281053 | 312 | 4 | 5.40E−04 | 0.042 | NA |
| chr18 | 47825004 | 47825241 | 238 | 4 | 8.65E−04 | 0.024 | NA |
| chr10 | 1.01E+08 | 1.01E+08 | 398 | 4 | 3.15E−05 | 0.034 | NA |
| chr6 | 1.7E+08 | 1.7E+08 | 469 | 4 | 1.33E−04 | 0.015 | NA |
| chr19 | 39754498 | 39754974 | 477 | 4 | 4.15E−04 | 0.020 | IFNL4P1 |
| chr11 | 94883474 | 94883722 | 249 | 3 | 7.26E−04 | 0.037 | RP11 |
| chr13 | 79234251 | 79234435 | 185 | 3 | 1.99E−04 | −0.081 | RNF219 |
| chr19 | 45885800 | 45885940 | 141 | 3 | 7.81E−04 | 0.029 | PPP1R13L |
| chr10 | 49812686 | 49812963 | 278 | 3 | 4.28E−04 | 0.030 | ARHGAP22 |
| chr11 | 14402556 | 14402815 | 260 | 3 | 8.65E−04 | 0.009 | NA |
| chr18 | 43418829 | 43419102 | 274 | 2 | 4.38E−04 | 0.047 | SIGLEC15 |
| chr4 | 1.16E+08 | 1.16E+08 | 15 | 2 | 1.52E−04 | 0.030 | UGT8 |
| chr7 | 27231491 | 27231819 | 329 | 2 | 8.73E−04 | 0.049 | RP1 |
| chr8 | 1.41E+08 | 1.41E+08 | 56 | 2 | 2.85E−04 | 0.029 | NA |
| chr15 | 84322946 | 84323154 | 209 | 2 | 6.99E−04 | 0.021 | ADAMTSL3 |
| chr7 | 1.2E+08 | 1.2E+08 | 27 | 2 | 7.27E−04 | 0.016 | KCND2 |
| IBS-C vs IBS-D | | | | | | | |
| chr7 | 27182493 | 27185512 | 3020 | 50 | 3.98E−17 | −0.024 | HOXA5 |
| chr6 | 31690725 | 31692300 | 1576 | 34 | 3.98E−17 | −0.016 | C6orf25 |
| chr20 | 57582581 | 57583709 | 1129 | 21 | 4.58E−07 | −0.020 | CTSZ |
| chr6 | 31539539 | 31540461 | 923 | 19 | 4.80E−06 | 0.041 | LTA |
| chr6 | 30079090 | 30079662 | 573 | 17 | 2.42E−04 | 0.029 | TRIM31 |
| chr10 | 1.25E+08 | 1.25E+08 | 1061 | 16 | 6.30E−08 | 0.026 | RP11 |
| chr7 | 27186993 | 27188770 | 1778 | 15 | 5.20E−06 | −0.022 | HOXA6 |
| chr5 | 1.26E+08 | 1.26E+08 | 798 | 13 | 1.69E−09 | 0.046 | C5orf63 |
| chr19 | 52390810 | 52391480 | 671 | 12 | 1.14E−04 | −0.038 | ZNF577 |
| chr1 | 75198403 | 75199117 | 715 | 10 | 1.93E−05 | 0.021 | CRYZ |
| chr11 | 94278324 | 94279068 | 745 | 10 | 7.14E−05 | −0.025 | FUT4 |
| chr2 | 1.57E+08 | 1.57E+08 | 1422 | 10 | 5.88E−08 | −0.028 | NR4A2 |
| chr6 | 74103959 | 74104868 | 910 | 10 | 1.49E−12 | 0.035 | DDX43 |
| chr15 | 91473059 | 91473569 | 511 | 9 | 3.25E−04 | 0.036 | UNC45A |
| chr16 | 53406901 | 53407808 | 908 | 9 | 1.30E−07 | −0.052 | RP11 |
| chr17 | 29297391 | 29297604 | 214 | 8 | 8.65E−05 | −0.039 | RNF135 |
| chr5 | 23507030 | 23507656 | 627 | 8 | 1.99E−04 | 0.032 | PRDM9 |
| chr6 | 42928056 | 42928303 | 248 | 8 | 8.74E−04 | −0.022 | GNMT |
| chr8 | 1.45E+08 | 1.45E+08 | 682 | 8 | 1.07E−04 | −0.026 | PLEC |
| chr17 | 73874443 | 73874790 | 348 | 7 | 9.45E−05 | −0.009 | TRIM47 |
| chr2 | 2.33E+08 | 2.33E+08 | 563 | 7 | 9.10E−06 | 0.036 | CHRND |
| chr20 | 30072314 | 30073576 | 1263 | 7 | 6.82E−07 | 0.047 | LINC00028 |
| chr5 | 8457548 | 8458392 | 845 | 7 | 8.96E−05 | 0.049 | RP11 |
| chr7 | 27197239 | 27198025 | 787 | 7 | 1.27E−05 | −0.016 | HOXA7 |
| chr11 | 504551 | 504937 | 387 | 6 | 4.69E−05 | −0.037 | RNH1 |
| chr4 | 1.47E+08 | 1.47E+08 | 472 | 6 | 5.61E−05 | 0.028 | RP11 |
| chr6 | 32808504 | 32808752 | 249 | 6 | 2.04E−04 | 0.021 | TAP2 |
| chr7 | 1.05E+08 | 1.05E+08 | 383 | 6 | 2.41E−05 | −0.031 | ATXN7L1 |
| chr3 | 52813652 | 52813920 | 269 | 6 | 2.42E−06 | −0.016 | ITIH1 |
| chr1 | 11795746 | 11795946 | 201 | 5 | 7.76E−04 | −0.028 | AGTRAP |
| chr12 | 6658378 | 6658945 | 568 | 5 | 1.54E−04 | −0.022 | IFFO1 |
| chr14 | 1.06E+08 | 1.06E+08 | 1004 | 5 | 5.20E−06 | 0.043 | TMEM121 |
| chr2 | 2.32E+08 | 2.32E+08 | 873 | 5 | 4.60E−07 | 0.039 | NCL |
| chr21 | 45773569 | 45774664 | 1096 | 5 | 1.14E−07 | −0.046 | TRPM2 |
| chr22 | 46285638 | 46285985 | 348 | 4 | 1.82E−04 | 0.056 | |
| chr5 | 1.41E+08 | 1.41E+08 | 402 | 4 | 4.72E−04 | −0.027 | PCDHGA12 |
| chr6 | 70991156 | 70991283 | 128 | 4 | 1.08E−04 | 0.024 | COL9A1 |
| chr8 | 53852030 | 53852274 | 245 | 4 | 4.93E−04 | −0.005 | NPBWR1 |
| chr6 | 31695027 | 31695415 | 389 | 4 | 3.77E−05 | −0.020 | DDAH2 |
| chr19 | 55874500 | 55874851 | 352 | 4 | 9.10E−05 | 0.017 | FAM71E2 |
| chr1 | 2.44E+08 | 2.44E+08 | 67 | 3 | 3.32E−04 | 0.038 | |
| chr11 | 1283875 | 1283970 | 96 | 3 | 4.08E−04 | 0.060 | |
| chr12 | 1.07E+08 | 1.07E+08 | 167 | 3 | 1.27E−05 | 0.039 | C12orf23 |
| chr16 | 88846234 | 88846723 | 490 | 3 | 9.67E−06 | −0.044 | |
| chr3 | 1.33E+08 | 1.33E+08 | 138 | 3 | 7.19E−05 | 0.035 | BFSP2 |
| chr5 | 42924215 | 42924552 | 338 | 3 | 7.96E−05 | 0.058 | |
| chr5 | 79553267 | 79553606 | 340 | 3 | 7.82E−05 | −0.029 | SERINC5 |
| chr7 | 1.55E+08 | 1.55E+08 | 285 | 3 | 1.24E−04 | 0.025 | |
| chr8 | 37698900 | 37699360 | 461 | 3 | 5.10E−04 | −0.037 | |
| chr12 | 1.33E+08 | 1.33E+08 | 99 | 2 | 1.50E−04 | −0.024 | |
| chr19 | 56002240 | 56002446 | 207 | 2 | 3.34E−05 | 0.020 | |
| chr2 | 10344980 | 10345003 | 24 | 2 | 4.23E−05 | −0.037 | |
| chr2 | 70312492 | 70312615 | 124 | 2 | 5.71E−05 | 0.066 | PCBP1 |
| chr5 | 1.57E+08 | 1.57E+08 | 128 | 2 | 7.20E−04 | 0.040 | AC008694.3 |

Legend: The Table shows differentially methylated regions (DMRs) associated IBS bowel habit (IBS-C, constipation; IBS-D, diarrhea; IBS-M, mixed) subtypes compared to healthy controls (HCs) and between IBS-C and IBS-D, in PBMCs. FDR, false detection rate, MeanBetaFC, average methylation beta value fold change.

DNA Methylation Based Subtypes within IBS

Clustering of 5000 most variant DNA methylation probes in PBMCs did not result in significant methylation-based subgroups within IBS.

### DNA Methylation Profile of IBS Patients Compared to HCs in Colonic Mucosa

Methylation Differences Between IBS Vs HCs

Sigmoid colonic mucosal biopsies showed no significant DMPs between IBS and HCs after correcting for multiple tests. Among the top hypermethylated sites in IBS compared to HCs were 15 suggestive DMPs (p<5.0E-05) that included CpG sites in KRIT1 and NPHP4 genes which are associated with ‘cell-adhesion’ and ‘cell-cell junctions’, respectively. Additionally, genes associated with GO terms such as ‘osmotic stress’ (TSC22D2), ‘response to stress’ (TP53TG1) and ‘oxidation-reduction process’ (BLVRB) were hypomethylated in IBS. mRNA transport, ion-transport (specifically, potassium ion transport) and cell-adhesion were among the top GO terms associated with the 231 sites differentially methylated at p<0.001, as shown in Table 2.

We found a significant association (FDR<0.05) of DMRs in the promoter regions of 7 genes in IBS compared to HCs. These DMRs were associated with genes involved in calcium ion homeostasis (stanniocalcin 2, STC2), transport of neutral amino acids and sodium ions (Sodium-coupled neutral amino acid transporter 4, SLC38A4) and myelin membrane lipid break down Galactocerebrocide (GALC). Stanniocalcinin (STC2) gene, had 3 consecutive CpGs, and Sodium-coupled neutral amino acid transporter 4 (SLC38A4) gene had 7 CpGs, hyper-methylated in IBS compared to healthy controls in colon. All the promoters DMRs were hypermethylated in IBS compared to HCs (Table 3) except the ones in Deoxyguanosine Kinase (DGUOK) and Sperm tail PG-rich repeat containing (STPG2).

Methylation of stress-related genes in IBS compared to HCs

Although no enrichment of differentially methylated stress-related genes was seen in the colonic mucosa, we observed significant correlations with IBS clinical traits. Increased methylation of cg00029973 in the promoter region of transient receptor potential vanilloid 1 (TRPV1), which is associated with hyperalgesia, was associated with lower PSS (perceived stress, FDR<0.05, FIG. 1). Additionally, promoter CpGs in cannabinoid receptor 1 (CNR1) and FK506 Binding Protein 4 (FKBP4) were negatively and positively correlated with PHQ-15 (somatic symptom severity) score, respectively.

Methylation differences between IBS bowel habit subtypes

Although there were no DMPs with FDR<0.05 between bowel habit subtypes, there were promoter DMRs associated with IBS bowel habits compared to HCs, which are listed in Table 7. Methylation differences between IBS-C and HCs included unc-45 myosin chaperone A (UNC45A, hypermethylated, 9 CpG sites), which acts as a regulator of the progesterone receptor chaperoning pathway, MIR4458HG (hypermethylated, 7 CpG sites), long non coding RNA: AL645941.1 (hypomethylated 6CpG sites), 5-hydroxytryptamine receptor 5B3,pseudogene (HTR5BP, hypermethylated, 5CpG sites) and STPG2 (hypomethylated, 4CpG sites).

TABLE 7
IBS bowel habit subtype associated differentially methylated regions in the colon.

| Chromosome | Start | End | Width | No. cpgs | Minfdr | Meanbetafc | Gene Symbol |
|---|---|---|---|---|---|---|---|
| IBS-C vs HC | | | | | | | |
| chr15 | 91473059 | 91473569 | 511 | 9 | 1.26E−08 | 0.044 | UNC45A |
| chr5 | 8457548 | 8458392 | 845 | 7 | 1.26E−08 | 0.055 | RP11-480D4.1 |
| chr6 | 32904889 | 32905320 | 432 | 6 | 2.21E−04 | −0.019 | HLA-DMB |
| chr2 | 1.19E+08 | 1.19E+08 | 422 | 5 | 8.40E−06 | 0.052 | HTR5BP |
| chr4 | 99064603 | 99064904 | 302 | 4 | 6.16E−04 | −0.026 | STPG2 |
| IBS-D vs HC | | | | | | | |
| chr6 | 29521220 | 29521604 | 385 | 16 | 9.41E−04 | −0.031 | OR2I1P |
| chr11 | 34460298 | 34460789 | 492 | 8 | 9.41E−04 | 0.026 | CAT |
| chr7 | 1.58E+08 | 1.58E+08 | 212 | 3 | 9.41E−04 | 0.040 | NA |
| IBS-M vs HC | | | | | | | |
| chr11 | 34460107 | 34461028 | 922 | 13 | 1.78E−05 | 0.029 | CAT |
| chr14 | 88459216 | 88459963 | 748 | 8 | 7.24E−06 | 0.023 | GALC |
| chr2 | 74153795 | 74154363 | 569 | 8 | 1.78E−05 | −0.019 | DGUOK |
| chr6 | 32120584 | 32120878 | 295 | 8 | 9.97E−05 | 0.033 | PRRT1 |
| chr8 | 1.44E+08 | 1.44E+08 | 601 | 8 | 1.76E−04 | 0.058 | C8orf31 |
| chr5 | 1.1E+08 | 1.1E+08 | 454 | 6 | 9.32E−05 | 0.083 | TMEM232 |
| chr10 | 530836 | 531320 | 485 | 5 | 2.32E−04 | −0.045 | NA |
| chr8 | 24151472 | 24151625 | 154 | 3 | 7.47E−04 | −0.066 | ADAM28 |
| IBS-C vs IBS-D | | | | | | | |
| chr6 | 74104388 | 74104868 | 481 | 6 | 1.48E−04 | 0.053 | DDX43 |
| chr6 | 31276504 | 31276669 | 166 | 4 | 4.13E−04 | 0.039 | XXbac- |
| | | | | | | | BPG248L24.10 |
| chr12 | 1.33E+08 | 1.33E+08 | 257 | 3 | 4.44E−04 | −0.042 | GALNT9 |
| chr5 | 8457818 | 8458089 | 272 | 3 | 4.86E−04 | 0.033 | RP11-480D4.1 |

Legend: The Table shows differentially methylated regions (DMRs) associated IBS bowel habit (IBS-C, constipation; IBS-D, diarrhea; IBS-M, mixed) subtypes compared to healthy controls (HCs) and between IBS-C and IBS-D in colon. FDR, false detection rate, MeanBetaFC, average methylation beta value fold change.

DMRs associated with IBS-D compared to HCs included Olfactory Receptor Family 2 Subfamily I Member 1Pseudogene (ORI1P, hypomethylated at 16CpG sites in IBS-D) and Catalase, which converts hydrogen peroxide to water and molecular oxygen (CAT, hypermethylated at 8CpG sites in IBS-D).We found DMRs in 8genes, including, CAT, GALC, DGUOK, Proline-ich transmembrane protein 1 (PRRT1), Transmembrane protein 232 (TMEM232) and A disintegrin and metalloprotease domain (ADAM28), associated with IBS-M compared to HCs.

DNA Methylation Based Subtypes within IBS

Clustering of 5000 most variant CpGs in the colonic mucosa within IBS revealed 3 methylation-based clusters (Cluster 1[N=26], Cluster 2[N=44], Cluster 3[N=32]). Compared to the other two clusters, Cluster 1was comprised predominantly of men and was associated with higher age, BMI, abdominal pain, and overall symptom severity (ANOVA p <0.05, FIG. 3). Even after controlling for age and sex, Cluster 1 remained significantly associated with abdominal pain and overall symptom severity (p=0.034 and p=0.006, respectively). A methylation signature of 2964 CpG sites (FDR<0.05, mean difference >3%) differentiated the two clusters showing divergent IBS symptoms (Cluster 1 vs 3). GO analysis revealed an enrichment of ion channel and neurotransmitter transport genes in the differentially methylated sites (FDR 0.05). Table shows the list of genes, hypermethylated in Cluster 1compared to Cluster 3that have been studied in the context of IBS.

TABLE 8
Genes hyper-methylated in Cluster 1 vs Cluster 3 that were
previously associated with IBS or IBS endophenotypes

| Gene | | | | |
|---|---|---|---|---|
| Symbol | Gene Name | MD (%) | FDR | Function |
| VIP | vasoactive intestinal peptide | 5.8 | 9.00E−04 | Along with mast cells, regulate |
| | | | | passage of colonic bacteria |
| TRPV4 | transient receptor potential | 5.7 | 2.11E−06 | Permeability/cell-cell junctions |
| | cation channel, subfamily V, | | | |
| | member 4 | | | |
| HTR7 | 5-hydroxytryptamine (serotonin) | 5.6 | 1.64E−11 | Nominally significant SNP |
| | receptor 7, adenylate cyclase- | | | associations |
| | coupled | | | |
| HTR5A | 5-hydroxytryptamine (serotonin) | 5.9 | 1.24E−06 | Nominally significant SNP |
| | receptor 5A, G protein-coupled | | | associations |
| SNCAIP | synuclein, alpha interacting | 6 | 1.37E−06 | Differentially methylated in pilot |
| | protein | | | methylation data in PBMCs of IBS |
| DRD1 | dopamine receptor D1 | 6.5 | 2.50E−10 | cAMP activation behavioral |
| | | | | response |
| GRID2IP | In multiple Geneids | 6.8 | 1.70E−02 | IBS GWAS |
| FGF12 | fibroblast growth factor 12 | 6.9 | 1.41E−06 | Bile acid biosynthesis |
| FGF10 | fibroblast growth factor 10 | 7 | 2.63E−06 | Bile acid biosynthesis |
| FGF3 | fibroblast growth factor 3 | 8.6 | 6.00E−04 | Bile acid biosynthesis |
| FGF4 | fibroblast growth factor 4 | 11 | 4.00E−04 | Bile acid biosynthesis |
| GUCY1B3 | guanylate cyclase 1, soluble, | 8.6 | 4.00E−02 | Guanylate cyclase activity cGMP |
| | beta 3 | | | biosynthesis |
| GUCA1A | guanylate cyclase activator 1A | 5.6 | 3.00E−03 | Guanylate cyclase activity cGIMP |
| | | | | biosynthesis |
| CDH4 | cadherin 4, type 1, R-cadherin | 22.8 | 1 00E−04 | Permeability/cell-cell junctions |
| CDH11 | cadherin 11, type 2, OB- | 10.9 | 2.00E−04 | Permeability/cell-cell junctions |
| | cadherin | | | |

Legend: This Table lists all the genes that were hyper-methylated in Cluster 1 compared to Cluster 3 within IBS which have been associated with IBS previously, along with their suggested function and/or relevance to IBS. MD, mean difference in methylation between clusters; FDR, false detection rate.

Identification of Co-Methylation Modules in IBS with WGCNA Associated with Clinical Traits

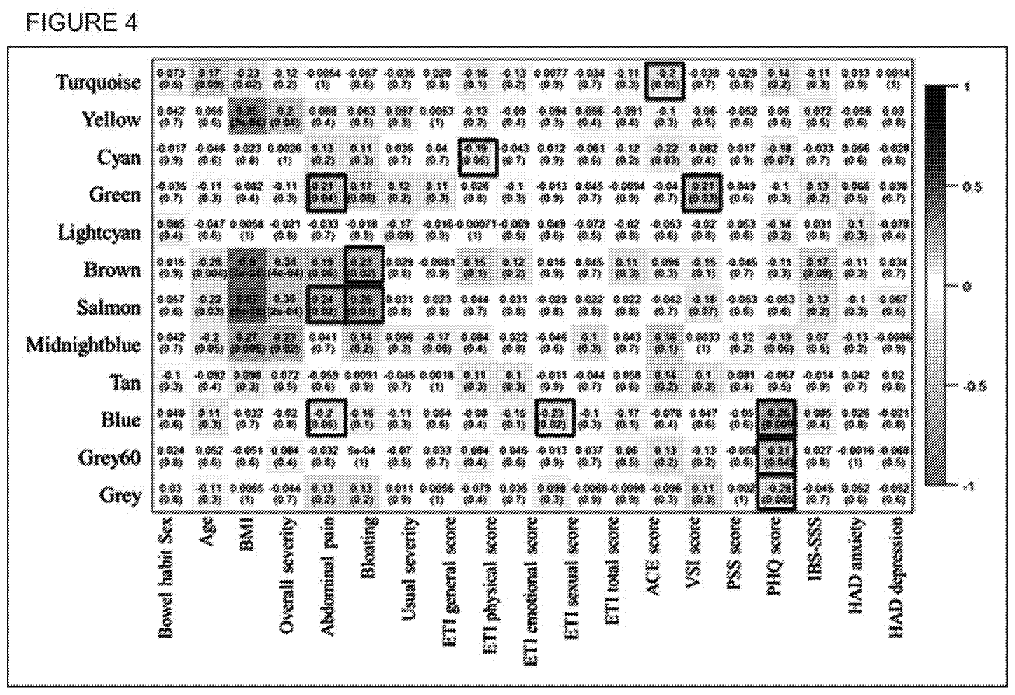

Next, we wanted to identify genes with similar methylation patterns and their association with clinical traits. Using WGCNA, we identified consensus modules grouped by methylation probes that are co-methylated in IBS and HCs. Twelve modules were significantly correlated with one or more clinical traits (FIG. 4). Module-trait associations along with the GO terms associated with the genes in the modules are listed in Table 9.

TABLE 9
Co-methylation modules associated with clinical features of IBS.

| | Associated Clinical | | |
|---|---|---|---|
| Module | Trait | Associated GO term | FDR % |
| Turquoise | ACE | Guanine-nucleotide releasing factor | 4.00E−03 |
| | | Cell-cell adherens junction | 1.9 |
| | | Sodium transport | 0.5 |
| Cyan | ACE | Protein sumoylation | 15.1 |
| | | Transmembrane helix | 23.2 |
| | | Olfactory Transduction | 33.7 |
| Green | VSI_Score | Vascular smooth muscle contraction | 4.75 |
| | | Synapse | 5.38 |
| Brown | Abdominal Pain | Cadherin, N-terminal; Cell adhesion | 1.08E−06 |
| | | Lipoprotein | 3.6 |
| | | Cell junciton | 2.3 |
| | | Ion channel | 2.6 |
| Salmon | Abdominal Pain | Synapse | 8.10E−09 |
| | | Domain: Cadherin 5 | 1.86E−05 |
| | | Ionotropic glutamate receptor activity | 3.57E−04 |
| | | Sensory perception of pain | 1.09E+00 |
| Blue | PHQ-15 | Mitochondrial inner membrane | 2.19E+00 |
| | | Lipid biosynthesis | 5.70E+00 |

Legend: This Table shows co-methylation modules associated with the clinical features of IBS and the associated gene ontology (GO) terms at false detection rate (FDR) <5%. ACE, adverse childhood events; VSI, visceral sensitivity index, PHQ; patent health questionnaire.

Brown and Salmon modules were associated with age, BMI, overall severity and abdominal pain and bloating and associated with GO terms such as ‘Cadherin’, ‘cell adhesion’ and ‘sensory perception of pain’ among others. However, when age was included as covariate in the model, the brown module or salmon modules were not associated with overall severity, abdominal pain or bloating. Glutaminergic synapse signaling pathway, which plays an important role in excitatory synaptic transmission and in pain, was among the most significant pathways associated with 522 genes in Salmon module and Glutamate Ionotropic Receptor Kainate Type Subunit 2 (GR/K2) showed highest intra-modular connectivity.

Gene Expression Changes in Colonic Mucosa Associated with IBS

There were three differentially expressed genes between IBS and HCs, and one differentially expressed gene between IBS-C vs HCs (FDR <0.05). These included; mitochondrially encoded NADH:ubiquinone oxidoreductase core subunit 2 pseudogene 28 (MTND2P28), which was significantly downregulated in overall IBS and IBS-C compared to HCs, and coronin, actin binding protein, 1A (CORO1A), significantly downregulated in IBS compared to HCs. Functional annotation clustering of 172 genes with a p<0.005 between IBS-D and HCs suggested association of terms including ‘Immunity’ and ‘inflammatory response’. Between IBS-C and HCs, 159 differentially expressed genes (p<0.005) were associated with terms including, ‘lectin or carbohydrate binding’ and ‘calmodulin binding’.

### Correlation Between DNA Methylation and Gene Expression

Correlation Between Methylation and Expression in all Subjects

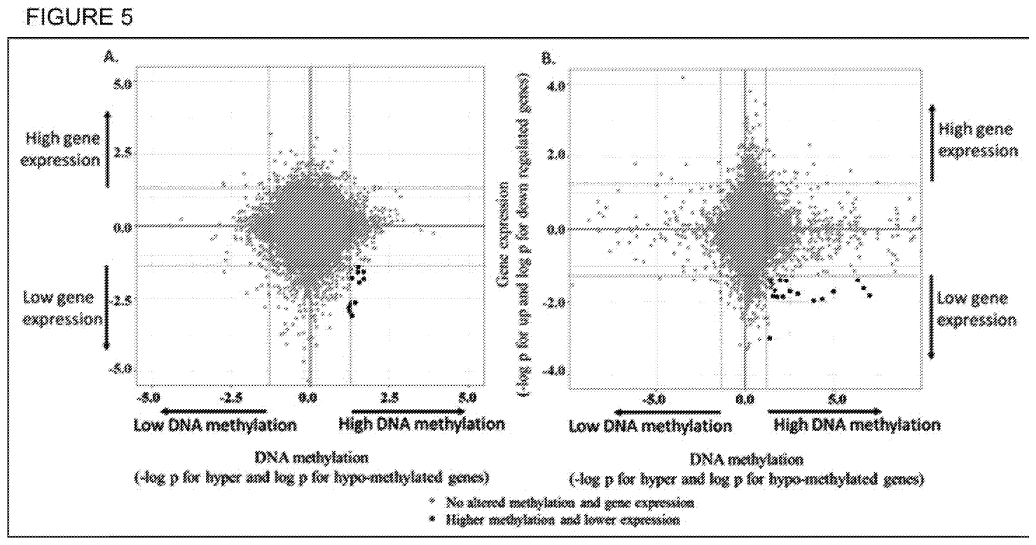

Spearman correlation of methylation for the all annotated CpG sites, and gene expression (probe with highest absolute fold change between IBS and HCs) identified methylation-related silencing in 160 genes (FDR<0.05). These genes (Table 10) included those involved in cell adhesion, such as the intestinal stem cell marker olfactomedin 4 (OLFM4) and Claudin 8 (CLDN8), and ion binding proteins and transporters such as solute carrier family 28 A, member 2 (SLC28A2) and solute carrier family 22 A member 18 antisense (SLC22A18AS). Using Starburst plot for investigating significant alterations associated with IBS compared to HCs in DNA methylation and gene expression, we identified 8 genes (FIG. 5A) that were significantly hypermethylated and downregulated in IBS compared to HCs (p<0.05) of which included transcription factors such as, elongation factor E2F Transcription Factor 3 (E2F3) and Homeobox protein Hox-D11 (HOXD11).

TABLE 10
DNA methylation-related silencing of gene expression in colonic mucosa

| | | | Spearman's | | |
|---|---|---|---|---|---|
| CpG site | GeneSymbol | Gene Name | Rho | P | adjp |
| cg24932628 | OLFM4 | olfactomedin 4 | −0.574 | <2.2e−16 | <2.2e−16 |
| cg01789267 | SLC28A2 | solute carrier family 28 | −0.722 | 9.99E−23 | 1.17E−17 |
| | | (concentrative nucleoside | | | |
| | | transporter), member 2 | | | |
| cg19305227 | SLC28A2 | solute carrier family 28 | −0.710 | 1.07E−21 | 8.39E−17 |
| | | (concentrative nucleoside | | | |
| | | transporter), member 2 | | | |
| cg01433189 | SLC28A2 | solute carrier family 28 | −0.707 | 1.88E−21 | 1.10E−16 |
| | | (concentrative nucleoside | | | |
| | | transporter), member 2 | | | |
| cg20995778 | NLRP2 | NLR family, pyrin domain | −0.602 | 1.77E−14 | 8.31E−10 |
| | | containing 2 | | | |
| cg01444765 | C21orf33 | In multiple Geneids | −0.555 | 4.23E−12 | 1.65E−07 |
| cg12582008 | OLFM4 | olfactomedin 4 | −0.529 | 3.37E−11 | 1.13E−06 |
| cg07222908 | SLC28A2 | solute carrier family 28 | −0.500 | 8.71E−10 | 2.27E−05 |
| | | (concentrative nucleoside | | | |
| | | transporter), member 2 | | | |
| cg04511125 | THNSL2 | threonine synthase-like 2 | −0.477 | 6.64E−09 | 1.44E−04 |
| cg07952391 | THNSL2 | threonine synthase-like 2 | −0.464 | 1.84E−08 | 3.59E−04 |
| cg19649172 | INPP5A | inositol polyphosphate-5- | −0.468 | 2.02E−08 | 3.64E−04 |
| | | phosphatase, 40 kDa | | | |
| cg24977027 | THNSL2 | threonine synthase-like 2 | −0.451 | 5.15E−08 | 8.05E−04 |
| cg02621376 | MCOLN2 | mucolipin 2 | −0.454 | 5.83E−08 | 8.55E−04 |
| cg15056348 | INPP5A | inositol polyphosphate-5- | −0.453 | 6.28E−08 | 8.67E−04 |
| | | phosphatase, 40 kDa | | | |
| cg08479294 | PDPR | pyruvate dehydrogenase | −0.440 | 1.54E−07 | 1.72E−03 |
| | | phosphatase regulatory | | | |
| | | subunit | | | |
| cg04739485 | MLXIP | MLX interacting protein | −0.439 | 1.68E−07 | 1.79E−03 |
| cg06376402 | INPP5A | inositol polyphosphate-5- | −0.438 | 1.77E−07 | 1.81E−03 |
| | | phosphatase, 40 kDa | | | |
| cg01013890 | MTSS1 | metastasis suppressor 1 | −0.429 | 2.55E−07 | 2.41E−03 |
| cg01413809 | MCOLN2 | mucolipin 2 | −0.433 | 2.56E−07 | 2.41E−03 |
| cg21907914 | ZNF677 | zinc finger protein 677 | −0.428 | 2.74E−07 | 2.45E−03 |
| cg12417955 | INPP5A | inositol polyphosphate-5- | −0.432 | 2.82E−07 | 2.45E−03 |
| | | phosphatase, 40 kDa | | | |
| cg22153463 | MCOLN2 | mucolipin 2 | −0.431 | 2.96E−07 | 2.48E−03 |
| cg18059223 | NLRP2 | NLR family, pyrin domain | −0.426 | 3.11E−07 | 2.51E−03 |
| | | containing 2 | | | |
| cg07819160 | MPST | mercaptopyruvate | −0.429 | 3.41E−07 | 2.67E−03 |
| | | sulfurtransferase | | | |
| cg19026320 | CLDN8 | claudin 8 | −0.424 | 3.78E−07 | 2.86E−03 |
| cg16571786 | RNF157 | ring finger protein 157 | −0.422 | 4.20E−07 | 3.03E−03 |
| cg05321225 | ASPG | asparaginase | −0.422 | 4.27E−07 | 3.03E−03 |
| cg22501243 | MLXIP | MLX interacting protein | −0.420 | 6.40E−07 | 4.17E−03 |
| cg26445292 | CACNB4 | calcium channel, voltage- | −0.406 | 1.25E−06 | 7.00E−03 |
| | | dependent, beta 4 subunit | | | |
| cg08047629 | GDPD3 | glycerophosphodiester | −0.405 | 1.35E−06 | 7.30E−03 |
| | | phosphodiesterase domain | | | |
| | | containing 3 | | | |
| cg09893465 | INPP5A | inositol polyphosphate-5- | −0.408 | 1.38E−06 | 7.30E−03 |
| | | phosphatase, 40 kDa | | | |
| cg16690845 | INPP5A | inositol polyphosphate-5- | −0.408 | 1.40E−06 | 7.30E−03 |
| | | phosphatase, 40 kDa | | | |
| cg02836764 | INPP5A | inositol polyphosphate-5- | −0.407 | 1.44E−06 | 7.37E−03 |
| | | phosphatase, 40 kDa | | | |
| cg18655522 | ACTN1 | actinin, alpha 1 | −0.406 | 1.58E−06 | 7.88E−03 |
| cg24175803 | ZNF257 | zinc finger protein 257 | −0.400 | 1.89E−06 | 9.06E−03 |
| cg11378575 | B3GNT7 | UDP-GIcNAc: betaGal beta- | −0.398 | 2.56E−06 | 1.14E−02 |
| | | 1,3-N- | | | |
| | | acetylglucosaminyltransferase | | | |
| | | 7 | | | |
| cg19321695 | TSPAN1 | tetraspanin 1 | −0.397 | 2.65E−06 | 1.14E−02 |
| cg05750788 | CORO1B | In multipie Geneids | −0.397 | 2.68E−06 | 1.14E−02 |
| cg07762788 | CDH1 | cadherin 1, type 1, E-cadherin | −0.397 | 2.80E−06 | 1.17E−02 |
| | | (epithelial) | | | |
| cg03966685 | CYP2W1 | cytochrome P450, family 2, | −0.392 | 2.99E−06 | 1.19E−02 |
| | | subfamily W, polypeptide 1 | | | |
| cg08443357 | SDCBP2 | syndecan binding protein | −0.395 | 3.00E−06 | 1.19E−02 |
| | | (syntenin) 2 | | | |
| cg09274988 | SDCBP2 | syndecan binding protein | −0.395 | 3.00E−06 | 1.19E−02 |
| | | (syntenin) 2 | | | |
| cg02335376 | GDPD3 | glycerophosphodiester | −0.392 | 3.08E−06 | 1.20E−02 |
| | | phosphodiesterase domain | | | |
| | | containing 3 | | | |
| cg12253469 | MPST | mercaptopyruvate | −0.394 | 3.19E−06 | 1.23E−02 |
| | | sulfurtransferase | | | |
| cg13878456 | IKZF3 | IKAROS family zinc finger 3 | −0.394 | 3.35E−06 | 1.25E−02 |
| cg14558114 | THNSL2 | threonine synthase-like 2 | −0.389 | 3.69E−06 | 1.35E−02 |
| cg04350136 | C6orf136 | chromosome 6 open reading | −0.388 | 3.93E−06 | 1.42E−02 |
| | | frame 136 | | | |
| cg00500048 | WFDC2 | WAP four-disulfide core | −0.391 | 4.01E−06 | 1.42E−02 |
| | | domain 2 | | | |
| cg11630148 | INPP5A | inositol polyphosphate-5- | −0.389 | 4.34E−06 | 1.52E−02 |
| | | phosphatase, 40 kDa | | | |
| cg24429836 | LDHD | lactate dehydrogenase D | −0.386 | 4.39E−06 | 1.52E−02 |
| cg27015174 | ADAL | adenosine deaminase-like | −0.389 | 4.47E−06 | 1.52E−02 |
| cg15336893 | CORO1B | In multiple Geneids | −0.388 | 4.79E−06 | 1.56E−02 |
| cg06574960 | B3GNT7 | UDP-GIcNAc: betaGal beta- | −0.387 | 5.05E−06 | 1.62E−02 |
| | | 1,3-N- | | | |
| | | acetylglucosaminyltransferase | | | |
| | | 7 | | | |
| cg03096107 | ZG16B | zymogen granule protein 16B | −0.383 | 5.35E−06 | 1.67E−02 |
| cg07926491 | STAT6 | signal transducer and | −0.385 | 5.61E−06 | 1.71E−02 |
| | | activator of transcription 6, | | | |
| | | interleukin-4 induced | | | |
| cg15225991 | INPP5A | inositol polyphosphate-5- | −0.385 | 5.63E−06 | 1.71E−02 |
| | | phosphatase, 40 kDa | | | |
| cg11262906 | MCOLN2 | mucolipin 2 | −0.385 | 5.69E−06 | 1.71E−02 |
| cg16173067 | SDCBP2 | syndecan binding protein | −0.384 | 5.82E−06 | 1.72E−02 |
| | | (syntenin) 2 | | | |
| cg04400540 | ATP6V0B | ATPase, H+ transporting, | −0.384 | 5.89E−06 | 1.72E−02 |
| | | lysosomal 21 kDa, V0 subunit | | | |
| | | b | | | |
| cg08491964 | TCF4 | transcription factor 4 | −0.384 | 5.93E−06 | 1.72E−02 |
| cg00924278 | ADI1 | acireductone dioxygenase 1 | −0.384 | 6.10E−06 | 1.75E−02 |
| cg05007126 | SDCBP2 | syndecan binding protein | −0.383 | 6.23E−06 | 1.75E−02 |
| | | (syntenin) 2 | | | |
| cg04420917 | LGALS4 | lectin, galactoside-binding, | −0.383 | 6.27E−06 | 1.75E−02 |
| | | soluble, 4 | | | |
| cg20965743 | ASPG | asparaginase | −0.379 | 6.75E−06 | 1.84E−02 |
| cg20656871 | ILKAP | integrin-linked kinase- | −0.381 | 7.33E−06 | 1.98E−02 |
| | | associated serine/threonine | | | |
| | | phosphatase | | | |
| cg04975920 | TSPAN1 | tetraspan in 1 | −0.379 | 7.93E−06 | 2.11E−02 |
| cg23464743 | HLA-DQB1 | major histocompatibility | −0.379 | 8.15E−06 | 2.15E−02 |
| | | complex, class II, DQ beta 1 | | | |
| cg04833731 | GDPD3 | glycerophosphodiester | −0.376 | 8.32E−06 | 2.15E−02 |
| | | phosphodiesterase domain | | | |
| | | containing 3 | | | |
| cg10548968 | CAPN5 | calpain 5 | −0.378 | 8.36E−06 | 2.15E−02 |
| cg14957346 | JUP | junction plakoglobin | −0.377 | 9.13E−06 | 2.25E−02 |
| cg10164272 | ANKRD11 | ankyrin repeat domain 11 | −0.377 | 9.19E−06 | 2.25E−02 |
| cg06230247 | MPST | mercaptopyruvate | −0.377 | 9.21E−06 | 2.25E−02 |
| | | sulfurtransferase | | | |
| cg21524538 | SDCBP2 | syndecan binding protein | −0.375 | 1.02E−05 | 2.42E−02 |
| | | (syntenin) 2 | | | |
| cg05590982 | NUPR1 | nuclear protein, transcriptional | −0.375 | 1.02E−05 | 2.42E−02 |
| | | regulator, 1 | | | |
| cg12126656 | INPP5A | inositol polyphosphate-5- | −0.375 | 1.04E−05 | 2.43E−02 |
| | | phosphatase, 40 kDa | | | |
| cg21521758 | RARRES2 | retinoic acid receptor | −0.372 | 1.07E−05 | 2.47E−02 |
| | | responder (tazarotene | | | |
| | | induced) 2 | | | |
| cg09004241 | B3GNT7 | UDP-GIcNAc: betaGal beta- | −0.374 | 1.09E−05 | 2.47E−02 |
| | | 1,3-N- | | | |
| | | acetylglucosaminyltransferase | | | |
| | | 7 | | | |
| cg26603685 | SULT1A1 | sulfotransferase family, | −0.374 | 1.10E−05 | 2.48E−02 |
| | | cytosolic, 1A, phenol- | | | |
| | | preferring, member 1 | | | |
| cg16019620 | MEP1A | meprin A, alpha (PABA | −0.371 | 1.11E−05 | 2.48E−02 |
| | | peptide hydrolase) | | | |
| cg03775632 | CALD1 | caldesmon 1 | −0.373 | 1.15E−05 | 2.51E−02 |
| cg22984282 | HLA-DQB1 | major histocompatibility | −0.373 | 1.16E−05 | 2.51E−02 |
| | | complex, class II, DQ beta 1 | | | |
| cg03449125 | CAPN5 | calpain 5 | −0.373 | 1.16E−05 | 2.51E−02 |
| cg01987333 | NEU4 | sialidase 4 | −0.372 | 1.19E−05 | 2.53E−02 |
| cg15120754 | ZEB2 | zinc finger E-box binding | −0.372 | 1.21E−05 | 2.54E−02 |
| | | homeobox 2 | | | |
| cg04066453 | JUP | junction plakoglobin | −0.372 | 1.22E−05 | 2.54E−02 |
| cg14807945 | ZEB2 | zinc finger E-box binding | −0.370 | 1.37E−05 | 2.75E−02 |
| | | homeobox 2 | | | |
| cg07217151 | ALG14 | ALG14, UDP-N- | −0.367 | 1.38E−05 | 2.75E−02 |
| | | acetylglucosaminyltransferase | | | |
| | | subunit | | | |
| cg27481594 | ADAL | adenosine deaminase-like | −0.370 | 1.38E−05 | 2.75E−02 |
| cg18335068 | ZNF677 | zinc finger protein 677 | −0.367 | 1.40E−05 | 2.75E−02 |
| cg02449202 | MEP1A | meprin A, alpha (PABA | −0.367 | 1.41E−05 | 2.75E−02 |
| | | peptide hydrolase) | | | |
| cg25755892 | PLA2G2A | phospholipase A2, group IIA | −0.368 | 1.50E−05 | 2.90E−02 |
| | | (platelets, synovial fluid) | | | |
| cg15975151 | ELMO1 | engulfment and cell motility 1 | −0.366 | 1.51E−05 | 2.90E−02 |
| cg03604278 | CIDEC | cell death-inducing DFFA-like | −0.364 | 1.61E−05 | 3.03E−02 |
| | | effector c | | | |
| cg05055712 | ZG16B | zymogen granule protein 16B | −0.364 | 1.69E−05 | 3.12E−02 |
| cg10807101 | GSTM3 | glutathione S-transferase mu | −0.363 | 1.77E−05 | 3.24E−02 |
| | | 3 (brain) | | | |
| cg01656853 | FUT2 | fucosyltransferase 2 (secretor | −0.363 | 1.78E−05 | 3.24E−02 |
| | | status included) | | | |
| cg21727129 | HLA-G | major histocompatibility | −0.362 | 1.81E−05 | 3.25E−02 |
| | | complex, class I, G | | | |
| cg22399646 | NEU4 | sialidase 4 | −0.365 | 1.83E−05 | 3.25E−02 |
| cg20299254 | WFDC2 | WAP four-disulfide core | −0.364 | 1.89E−05 | 3.30E−02 |
| | | domain 2 | | | |
| cg09616556 | AMN | amnion associated | −0.364 | 1.90E−05 | 3.30E−02 |
| | | transmembrane protein | | | |
| cg20980592 | MEP1A | meprin A, alpha (PABA | −0.361 | 1.94E−05 | 3.34E−02 |
| | | peptide hydrolase) | | | |
| cg06382664 | RELT | RELT tumor necrosis factor | −0.361 | 1.95E−05 | 3.34E−02 |
| | | receptor | | | |
| cg10146112 | INPP5A | inositol polyphosphate-5- | −0.363 | 2.04E−05 | 3.47E−02 |
| | | phosphatase, 40 kDa | | | |
| cg01140244 | INPP5A | inositol polyphosphate-5- | −0.362 | 2.14E−05 | 3.53E−02 |
| | | phosphatase, 40 kDa | | | |
| cg05532325 | B3GNT7 | UDP-GIcNAc: betaGal beta- | −0.360 | 2.32E−05 | 3.68E−02 |
| | | 1,3-N- | | | |
| | | acetylglucosaminyltransferase | | | |
| | | 7 | | | |
| cg02558476 | ZG16B | zymogen granule protein 16B | −0.358 | 2.33E−05 | 3.68E−02 |
| cg09249657 | RBM47 | RNA binding motif protein 47 | −0.360 | 2.34E−05 | 3.68E−02 |
| cg02855996 | REPIN1 | replication initiator 1 | −0.358 | 2.36E−05 | 3.68E−02 |
| cg04847795 | HHLA3 | HERV-H LTR-associating 3 | −0.357 | 2.42E−05 | 3.75E−02 |
| cg12911952 | SLC22A18AS | AS solute carrier family 22 | −0.357 | 2.45E−05 | 3.78E−02 |
| | | (organic cation transporter), | | | |
| | | member 18 antisense | | | |
| cg23382741 | PPP1R14D | protein phosphatase 1, | −0.357 | 2.47E−05 | 3.78E−02 |
| | | regulatory (inhibitor) subunit | | | |
| | | 14D | | | |
| cg04660111 | FUT2 | fucosyltransferase 2 (secretor | −0.357 | 2.49E−05 | 3.79E−02 |
| | | status included) | | | |
| cg07889936 | C6orf136 | chromosome 6 open reading | −0.356 | 2.54E−05 | 3.82E−02 |
| | | frame 136 | | | |
| cg06894011 | CKB | creatine kinase, brain | −0.359 | 2.55E−05 | 3.82E−02 |
| cg24886788 | PPP1R14D | protein phosphatase 1, | −0.356 | 2.56E−05 | 3.82E−02 |
| | | regulatory (inhibitor) subunit | | | |
| | | 14D | | | |
| cg02729303 | FAM134B | family with sequence | −0.358 | 2.61E−05 | 3.86E−02 |
| | | similarity 134, member B | | | |
| cg10249814 | MSI2 | musashi RNA-bindirtg protein | −0.358 | 2.63E−05 | 3.86E−02 |
| | | 2 | | | |
| cg06427867 | CLDN8 | claudin 8 | −0.356 | 2.63E−05 | 3.86E−02 |
| cg25404339 | ZNF677 | zinc finger protein 677 | −0.355 | 2.70E−05 | 3.87E−02 |
| cg23811268 | WDR35 | WD repeat domain 35 | −0.355 | 2.73E−05 | 3.87E−02 |
| cg18718349 | C6orf136 | chromosome 6 open reading | −0.355 | 2.74E−05 | 3.87E−02 |
| | | frame 136 | | | |
| cg11327659 | RARRES2 | retinoic acid receptor | −0.355 | 2.78E−05 | 3.89E−02 |
| | | responder (tazarotene | | | |
| | | induced) 2 | | | |
| cg16519477 | FAF1 | Fas (TNFRSF6) associated | −0.354 | 2.85E−05 | 3.94E−02 |
| | | factor 1 | | | |
| cg19792268 | TMTC1 | transmembrane and | −0.354 | 2.97E−05 | 4.00E−02 |
| | | tetratricopeptide repeat | | | |
| | | containing 1 | | | |
| cg02241363 | NPTX1 | neuronal pentraxin I | −0.354 | 2.99E−05 | 4.01E−02 |
| cg03202060 | HLA-DQB1 | major histocompatibility | −0.355 | 3.09E−05 | 4.11E−02 |
| | | complex, class II, DQ beta 1 | | | |
| cg11397548 | DNALI1 | dynein, axonemal, light | −0.353 | 3.10E−05 | 4.11E−02 |
| | | intermediate chain 1 | | | |
| cg06723057 | NUPR1 | nuclear protein, transcriptional | −0.355 | 3.15E−05 | 4.11E−02 |
| | | regulator, 1 | | | |
| cg08229522 | GON4L | gon-4-like | −0.354 | 3.20E−05 | 4.14E−02 |
| cg18207141 | MRPS18B | In multiple Geneids | −0.354 | 3.22E−05 | 4.15E−02 |
| cg23620049 | LIPH | lipase, member H | −0.354 | 3.24E−05 | 4.15E−02 |
| cg19426266 | ZEB2 | zinc finger E-box binding | −0.354 | 3.26E−05 | 4.16E−02 |
| | | homeobox 2 | | | |
| cg23010585 | CORO1B | In multiple Geneids | −0.354 | 3.30E−05 | 4.18E−02 |
| cg07378067 | AMN | amnion associated | −0.354 | 3.32E−05 | 4.18E−02 |
| | | transmembrane protein | | | |
| cg01370334 | ADI1 | acireductone dioxygenase 1 | −0.354 | 3.33E−05 | 4.18E−02 |
| cg11740099 | SECTM1 | secreted and transmembrane | −0.353 | 3.38E−05 | 4.22E−02 |
| | | 1 | | | |
| cg12620005 | TTC39A | tetratricopeptide repeat | −0.351 | 3.45E−05 | 4.28E−02 |
| | | domain 39A | | | |
| cg13174051 | INPP5A | inositol polyphosphate-5- | −0.353 | 3.54E−05 | 4.35E−02 |
| | | phosphatase, 40 kDa | | | |
| cg05990080 | CTDSPL | CTD (carboxy-terminal | −0.352 | 3.56E−05 | 4.35E−02 |
| | | domain, RNA polymerase II, | | | |
| | | polypeptide A) small | | | |
| | | phosphatase-like | | | |
| cg08818284 | NUPR1 | nuclear protein, transcriptional | −0.352 | 3.68E−05 | 4.46E−02 |
| | | regulator, 1 | | | |
| cg07150062 | ASPG | asparaginase | −0.350 | 3.73E−05 | 4.46E−02 |
| cg24926276 | LRG1 | leucine-rich alpha-2- | −0.349 | 3.79E−05 | 4.46E−02 |
| | | glycoprotein 1 | | | |
| cg19033308 | ID1 | inhibitor of DNA binding 1, | −0.351 | 3.83E−05 | 4.46E−02 |
| | | dominant negative helix-loop- | | | |
| | | helix protein | | | |
| cg09898695 | SPINT1 | serine peptidase inhibitor, | −0.349 | 3.84E−05 | 4.46E−02 |
| | | Kunitz type 1 | | | |
| cg23202887 | BCL11B | B-cell CLL/lymphoma 11B | −0.348 | 3.96E−05 | 4.53E−02 |
| | | (zinc finger protein) | | | |
| cg01972843 | CECR1 | cat eye syndrome | −0.350 | 3.97E−05 | 4.53E−02 |
| | | chromosome region, | | | |
| | | candidate 1 | | | |
| cg02719634 | SLC22A18AS | AS solute carrier family 22 | −0.348 | 3.98E−05 | 4.53E−02 |
| | | (organic cation transporter), | | | |
| | | member 18 antisense | | | |
| cg09596336 | ZEB2 | zinc finger E-box binding | −0.350 | 4.00E−05 | 4.53E−02 |
| | | homeobox 2 | | | |
| cg24024214 | BTNL8 | butyrophilin-like 8 | −0.348 | 4.02E−05 | 4.54E−02 |
| cg05991820 | ECHDC3 | enoyl CoA hydratase domain | −0.347 | 4.23E−05 | 4.68E−02 |
| | | containing 3 | | | |
| cg17593512 | RBM47 | RNA binding motif protein 47 | −0.349 | 4.36E−05 | 4.76E−02 |
| cg07565422 | PGPEP1 | In multiple Geneids | −0.348 | 4.45E−05 | 4.83E−02 |
| cg02155558 | ADAL | adenosine deaminase-like | −0.348 | 4.52E−05 | 4.86E−02 |
| cg10761315 | ASPG | asparaginase | −0.345 | 4.69E−05 | 4.95E−02 |
| cg15589126 | B3GNT7 | UDP-GIcNAc: betaGal beta- | −0.347 | 4.74E−05 | 4.95E−02 |
| | | 1,3-N- | | | |
| | | acetylglucosaminyltransferase | | | |
| | | 7 | | | |
| cg09235217 | B3GNT7 | UDP-GIcNAc: betaGal beta- | −0.347 | 4.80E−05 | 4.96E−02 |
| | | 1,3-N- | | | |
| | | acetylglucosaminyltransferase | | | |
| | | 7 | | | |
| cg08099797 | LIPH | lipase, member H | −0.347 | 4.84E−05 | 4.98E−02 |
| cg14547644 | ZSCAN23 | zinc finger and SCAN domain | −0.344 | 4.93E−05 | 4.99E−02 |
| | | containing 23 | | | |
| cg15729154 | SLC22A18AS | AS solute carrier family 22 | −0.344 | 4.95E−05 | 4.99E−02 |
| | | (organic cation transporter), | | | |
| | | member 18 antisense | | | |
| cg05461841 | ZG16B | zymogen granule protein 16B | −0.344 | 4.96E−05 | 4.99E−02 |

Legend: This Table shows the list of genes (n=160) whose methylation was significantly negatively correlated with gene expression in colonic mucosa of all subjects including IBS and healthy controls. FDR, false detection rate.

Integration of methylation and gene expression data for methylation based Clusters 1 and 3within IBS identified 25 genes whose methylation was significantly higher and gene expression significantly lower (p<0.05) in Cluster 1 (which was associated with higher abdominal pain) compared to Cluster 3,as shown in the starburst plot (FIG. 5B).These genes were enriched in functional clusters such as calcium ion binding, transcription and cell junction (Table 11).

TABLE 11
Significant DNA methylation and gene expression alterations
between Cluster 1 and Cluster 3 within IBS

| Gene | | | | | | Associated |
|---|---|---|---|---|---|---|
| Symbol | Gene name | FDR_M | MD_M | P_GE | FC_GE | GO terms |
| CDH11 | cadherin 11 | 0.000 | 0.109 | 0.036 | −1.553 | Calcium ion |
| | | | | | | binding |
| CLGN | calmegin | 0.001 | 0.058 | 0.013 | −2.240 | Calcium ion |
| | | | | | | binding |
| DOC2A | double C2 domain alpha | 0.015 | 0.110 | 0.001 | −3.013 | Calcium ion |
| | | | | | | binding, cell |
| | | | | | | junction |
| NELL2 | neural EGFL like 2 | 0.163 | 0.111 | 0.025 | −2.432 | Calcium ion |
| | | | | | | binding |
| DTX1 | deltex E3 ubiquitin ligase 1 | 0.000 | 0.068 | 0.032 | −1.926 | Transcription, |
| | | | | | | DNA-templated |
| ILF2 | interleukin enhancer binding | 0.222 | 0.008 | 0.041 | −1.208 | Transcription, |
| | factor 2 | | | | | DNA-templated |
| LYL1 | LYL1, basic helix-loop-helix | 0.035 | 0.085 | 0.035 | −1.805 | Transcription, |
| | family member | | | | | DNA-templated |
| NCOA7 | nuclear receptor coactivator 7 | 0.257 | 0.057 | 0.001 | −1.470 | Transcription, |
| | | | | | | DNA-templated |
| NFIX | nuclear factor I X | 0.003 | 0.110 | 0.040 | −1.346 | Transcription, |
| | | | | | | DNA-templated |
| ZNF677 | zinc finger protein 677 | 0.000 | 0.036 | 0.005 | −1.873 | Transcription, |
| | | | | | | DNA-templated |
| ZNF829 | zinc finger protein 677 | 0.206 | 0.055 | 0.043 | −1.739 | Transcription, |
| | | | | | | DNA-templated |
| DPM2 | dolichyl-phosphate | 0.015 | 0.014 | 0.049 | −1.385 | Membrane |
| | mannosyltransferase subunit | | | | | |
| | 2, regulatory | | | | | |
| DSTYK | dual serine/threonine and | 0.169 | 0.037 | 0.046 | −1.323 | Membrane, cell |
| | tyrosine protein kinase | | | | | junction |
| GJA4 | gap junction protein alpha 4 | 0.111 | 0.131 | 0.042 | −2.332 | Membrane, cell |
| | | | | | | junction |
| PPP2R2B | protein phosphatase 2 | 0.001 | 0.078 | 0.032 | −2.218 | Membrane |
| | regulatory subunit B | | | | | |
| BLOC1S3 | biogenesis of lysosomal | 0.042 | 0.046 | 0.036 | −1.564 | NA |
| | organelles complex-1 | | | | | |
| C1orf35 | chromosome 1 open reading | 0.001 | 0.048 | 0.042 | −1.445 | NA |
| | frame 35 | | | | | |
| COL14A1 | collagen, type XIV, alpha 1 | 0.002 | 0.038 | 0.003 | −1.576 | NA |
| DYRK4 | dual-specificity tyrosine-(Y)- | 0.274 | 0.053 | 0.010 | −1.696 | NA |
| | phosphorylation | | | | | |
| FAM160B2 | family with sequence similarity | 0.089 | 0.164 | 0.016 | −1.474 | NA |
| | 160 | | | | | |
| KIF15 | kinesin family member 15 | 0.051 | 0.123 | 0.033 | −1.583 | NA |
| MOAP1 | modulator of apoptosis 1 | 0.130 | 0.009 | 0.030 | −1.602 | NA |
| NACAD | NAC alpha domain containing | 0.052 | 0.054 | 0.038 | −2.063 | NA |
| RBM17 | RNA binding motif protein 17 | 0.158 | 0.081 | 0.032 | −1.317 | NA |
| SNORD89 | small nucleolar RNA, C/D box | 0.187 | 0.270 | 0.011 | −2.126 | NA |
| | 89 | | | | | |

Legend:Thetableshowsthegenesthatwerehyper-methylatedanddown-regulated in Cluster 1 compared to Cluster 3; FDR_M, FDR corrected p value for methylation differences between Cluster 1 and Cluster 3; MD_M, Mean methylation differences between Cluster 1 and Cluster 3; P_GE, P value for gene expression (GE) differences between Cluster 1 compared to Cluster 3; FC_GE, GE fold change between Cluster 1 and Cluster 3; GO, Gene Ontology.

Correlation Between Methylation Patterns in PBMCs and Colon

Of the 13,400 and 17,800 suggestive DMPs in PBMCs and colon respectively, in IBS patients, we found 543 (˜5%) genes that were differentially methylated both in PBMCs and colon. GO analysis of these genes suggested association of terms including ‘microtubule’ and ‘cell-cell adherends junction’.

### Discussion

This is the first study to our knowledge that comprehensively investigated genome-wide DNA methylation patterns in IBS and HCs in PBMCs and colon to identify IBS-specific methylation patterns. The main findings of this study are 1) IBS is associated with differentially methylated regions in genes associated with cell adhesion and ion transport in PBMCs and colon; 2) Enrichment of differentially methylated CpG in stress-related genes in PBMCs and the association of methylation of stress-related genes in the colon and PBMCs with extraintestinal clinical features suggests a role for these genes in linking stress with symptoms in IBS patients; 3) We identified a set of CpG sites that can potentially serve as a biomarker for the diagnosis of IBS; 4) There are methylation-based subtypes in the colonic mucosa of IBS patients which identify IBS endophenotypes; and 5) Methylation of certain CpG sites in cell-adhesion and ion-transport genes was seen in PBMCs and colon and may be important in the pathophysiology of IBS.

### REFERENCES

1. Longstreth G F, et al. Gastroenterology 2006; 130:1480-1491.
2. Lovell R M, Ford A C. Clin Gastroenterol Hepatol Off Clin Pract J Am Gastroenterol Assoc 2012; 10:712-721.e4.
3. Spiller R, et al. Gut 2007; 56:1770-1798.
4. Drossman D A, Hasler W L. Gastroenterology 2016; 150:1257-1261.
5. Chang L. Gastroenterology 2011; 140:761-765.
6. Bradford K, et al. Clin Gastroenterol Hepatol Off Clin Pract J Am Gastroenterol Assoc 2012:10:385-390.e1-3.
7. Park S H, et al. Neurogastroenterol Motil Off J Eur Gastrointest Motil Soc 2016; 28:1252-1260.
8. Esteller M. N Engl J Med 2008; 358:1148-1159.
9. Denk F, McMahon S B. Neuron 2012; 73:435-444.
10. Radley J J, et al. Stress Amst Neth 2011; 14:481-497.
11. Bird A. Perceptions of epigenetics. Nature 2007:447:396-398.
12. Gudsnuk K, Champagne F A. ILAR J 2012; 53:279-288.
13. Roth T L. Dev Psychopathol 2013; 25:1279-1291.
14. Bjomsson H T, et al. Trends Genet TIG 2004; 20:350-358.
15. Deaton A M, Bird A. Genes Dev 2011; 25:1010-1022.
16. Yang X, et al. Cancer Cell 2014:26:577-590.
17. Sproul D, Meehan R R. Brief Funct Genomics 2013; 12:174-190.
18. Lim Y C, et al. PloS One 2017; 12:e0181155.
19. Weaver I C G, et al. Nat Neurosci 2004; 7:847-854.
20. Prusator D K, et al. Pain 2017:158:296-305.
21. Hong S, et al. Gastroenterology 2015; 148:148-157.e7.
22. Palma-Gudiel H, et al. Epigenetics 2015; 10:893-902.
23. Waltes R, et al. Am J Med Genet B Neuropsychiatr Genet 2016; 171:650-675.
24. Meer D van der, et al. Transl Psychiatry 2017; 7:e1145.
25. Gerritsen L, et al. Neuropsychopharmacol Off Publ Am Coll Neuropsychopharmacol 2017; 42:2446-2455.
26. Zheleznyakova G Y, et al. Behav Brain Funct BBF 2016; 12:17.
27. Sailaja B S, et al. Proc Nati Acad Sci USA 2012; 109:E3687-3695.
28. Camilleri M, et al. Expert Rev Gastroenterol Hepatol 2017; 11:303-316.
29. Kim H, et al. J Genet Genomics Yi Chuan Xue Bao 2018; 45:87-97.
30. Suva M L, et al. Science 2013; 339:1567-1570.
31. Jenuwein T, Allis C D. Science 2001; 293:1074-1080.
32. Mahurkar S, et al. Neurogastroenterol Motil Off J Eur Gastrointest Motil Soc 2016; 28:410-422.
33. Sheehan D V, et al. J Clin Psychiatry 1998; 59 Suppl 20:22-33; quiz 34-57.
34. Munakata J, et al. Gastroenterology 1997; 112:55-63.
35. Zigmond A S, Snaith R P. Acta Psychiatr Scand 1983; 67:361-370.
36. Cohen S, et al. J Health Soc Behav 1983; 24:385-396.
37. Moll P, et al. Nat Methods 2014. Available at: https://www.nature.com/articles/nmeth.f.376 [Accessed Apr. 9, 2018].
38. N. L. Johnson, S. Kotz, A. W. Kemp. Univariate Discrete Distributions. Second Edition. New York: Wiley; 1992.
39. Fortin J-P, et al. Genome Biol 2014; 15:503.
40. Horvath S. Genome Biol 2013:14:R115.
41. Houseman E A, et al. BMC Bioinformatics 2012; 13:86.
42. Aryee M J, et al. Bioinforma Oxf Engl 2014; 30:1363-1369.
43. Peters T J, et al. Epigenetics Chromatin 2015; 8:6.
44. Liaw A, Wiener M. Classification and Regression by randomForest. R News 2002; 2:18-22.
45. Breiman L. Random forests. Mach Leam 2001; 45:5-32.
46. Robin X, et al. BMC Bioinformatics 2011; 12:77.
47. Youden W J. Cancer 1950; 3:32-35.
48. Wilkerson M D, Hayes D N. Bioinforma Oxf Engl 2010:26:1572-1573.
49. Love M I, et al. Genome Biol 2014; 15:550.
50. Huang D W, et al. Nat Protoc 2009:4:44-57.
51. Davis M E, Gack M U. Virology 2015; 479-480:52-65.
52. Han Y-F, Cao G-W. World J Gastroenterol 2012; 18:6865-6873.

### Supplementary Methods:

Bioinformatic and Statistical Analysis:

DNA methylation array. All the analyses were performed using R statistical analysis software using packages including but not limited to “lumi”, “methylumi” and “limma”. The oligomer probe designs of HM450 arrays follow the Infinium I and II chemistries, in which locus-specific base extension follows hybridization to a methylation-specific oligomer. The level of DNA methylation at each CpG locus was scored as beta (s) value calculated as (M/(M+U)), ranging from 0 to 1, with 0 indicating no DNA methylation and 1 indicating fully methylated DNA. Data was normalized using functional normalization, in order to preserve large tissue-related differences. Of the 485,577 CpG probes on the array, we filtered out probes with high detection p values (n=13326, p<0.01), cross reactive probes (probes with probes with at least 50 nucleotide homology [29], n=26058), probes with a SNP and repeat regions within 10 base pairs of the target CpG¹, n=15168), probes with no cg ids (n=65) and probes on X and Y chromosomes (n=10703), leaving 420257 probes for analysis. Bata values were converted to M-values using beta2M function from minfi package. Since no batch effects were observed on the clustering of 1000 most variant methylation probes, no further correction was applied. Abundance measures for various cell types including, plasma blasts, CD8+CD28-CD45RA-T cells, naive CD8 T cells, and naive CD4 T cells CD8 T cells, CD4 T cells, natural killer cells, B cells, monocytes and granulocytes was estimated in PBMCs of IBS patients and healthy controls using ‘The epigenetic clock’ software² which uses method and R code described by Houseman et al³. None of the estimated cell proportions were different between the two groups, therefore no adjustments were made.

The term ‘hyper-methylation’ was used when there was an increased DNA methylation in IBS patients (or bowel habit subtypes) compared to controls and, the term ‘hypo-methylation’ was used when we observed a decreased DNA methylation in IBS patients (or bowel habit subtypes) compared to controls. Differentially methylated regions (DMRs) were investigated using ‘DMRCate’ package⁴. Significant DMRs were defined based on three criteria. First, a DMR should contain more than one probe. Second, regional information can be combined from probes within 1000 basepairs (bp), which were defaults in DMRcate. Third, the region showed a p value <0.001 (as there was no significant DMP at FDR<0.05). A significant DMR can be detected even if there is no genome-wide significant DMP in the region⁵. For statistical significance, we set a threshold of FDR p<0.05 for significant DMPs and p<1.0×10⁻⁵, an arbitrary threshold, for suggestive DMPs, and p<0.001 for finding associated gene ontology (GO) terms. For genes with a priori hypotheses, for example, stress-related genes, a p<0.05 was used.

DNA methylation β-values for the selected probes on IBS and control samples were represented graphically by plotting heatmaps, generated using the R package ‘heatmap.plus’ (https://CRAN.R-project.org/package=heatmap.plus). DMRs were visualized using “Gviz” package.

WGCNA. The WGCNA⁷ R software was applied to methylation data after selecting the most variable methylation probe per gene, identified using ‘collapseRows’ function. Modules were identified for IBS and healthy controls separately using ‘blockwiseConsensusModules’ function using a soft power of 10. Each module was assigned a color, and a Module Eigengene (ME) corresponding to its first principal component, was calculated. The ME was correlated to IBS clinical traits, including, Age, Sex, BMI, overall severity of symptoms, abdominal pain, bloating, usual severity, early trauma inventory (ETI) physical, emotional, sexual, and total scores, to assess the significance of module-trait association (eigengene significance), adverse childhood effects (ACE) score, visceral sensitivity index (VSI), perceived stress score (PSS), somatization of symptoms measured by PHQ score, IBS symptom severity score (IBS-SSS), anxiety and depression scores.

Gene expression. QuantSeq library preparation: Normalized quantities of RNA were converted into cDNA by using QuantSeq 3′mRNA-Seq Reverse (REV) Library Prep Kit (Lexogen) according to manufacturer's instruction to generate compatible library for Illumina sequencing. cDNA libraries were assessed using TapeStation (Agilent Technologies, USA) before 100 bp single end sequencing using Illumina HiSeq 2500 system at UCLA Neuroscience Genomics Core based on standard protocols.

Quality trimming was performed to remove adapter sequences and polyA tails with parameter setting by following the quantseq data analysis user guide from Lexogen [1].

One probe with highest methylation difference between IBS and healthy controls was chosen and correlated with the gene expression probe with highest IBS-healthy control differences per gene, using ‘spearman correlation’, to identify highly negatively correlated, epigenetically silences genes in IBS.

### REFERENCES

1. Byun H-M, et al. Hum Mol Genet 2009:18:4808-4817.
2. Horvath S. Genome Biol 2013; 14:R115.
3. Houseman E A, et al. BMC Bioinformatics 2012:13:86.
4. Peters T J, et al. Epigenetics Chromatin 2015; 8:6.
5. Anon. Epigenome-wide association study of chronic obstructive pulmonary disease and lung function in Koreans.-PubMed-NCBI. Available at https://www.ncbi.nlm.nih.gov/pubmed/?term=Epigenome-wide+association+study+of+chronic+obstructive+pulmonary+disease+ and+lung+function+in +Koreans [Accessed Apr. 9, 2018].
6. Hahne F, Ivanek R. Methods Mol Biol Clifton N.J. 2016; 1418:335-351.
7. Langfelder P, Horvath S. BMC Bioinformatics 2008; 9:559.

### Example 2: DNA Methylation-Based Biomarkers in Blood for Differential Diagnosis of IBS from IBD

This Example demonstrates that methylation-based biomarkers that discriminate between IBS and IBD can be used for diagnosing IBS and ruling out IBD.

Irritable bowel syndrome (IBS) is a highly prevalent, chronic gastrointestinal (GI) disorder characterized by abdominal pain associated with diarrhea and/or constipation¹. Symptoms of IBS such as abdominal pain and bowel habit changes significantly overlap with other gastrointestinal (GI) conditions such as, inflammatory bowel disease (IBD)², a chronic relapsing inflammatory disorder, and celiac disease (CD)³, which is an autoimmune disorder characterized by intolerance to gluten. A few diagnostic biomarkers have been proposed in IBS⁴, however they perform modestly in predicting IBS. Moreover, there are no biomarkers that can distinguish IBS from IBD.

Diagnosis of IBS is a diagnosis of exclusion and in most cases additional tests are ordered, including stool studies to exclude infectious etiologies, IBD serologic panel, upper endoscopy and colonoscopy, abdominal CT scan, ultrasound, and breath test (to exclude small bacterial overgrowth), to rule out other conditions. DNA methylation marks have been proposed as diagnostic biomarkers in cancer⁵⁻⁷, however, they have not been explored in diagnosing IBS. Nonetheless, epigenetic marks can potentially serve as diagnostic biomarkers and also lend insight into the overlapping or divergent pathophysiological mechanisms of IBS and the diseases that mimic IBS symptoms. Therefore, the present study was aimed at investigating methylation-based biomarkers that discriminate between IBS and IBD, and can be used for diagnosing IBS and ruling out IBD.

### METHODS

Processed matrix data (normalized beta values) from HM450 Illumina array data on peripheral blood leukocytes (PBLs) of IBD patients was downloaded from GEO database (accession: GSE32148). Quantile DNA methylation data on IBS subjects was generated on the HM450 Illumina array on the peripheral blood mononuclear cells and quantile normalized. From the larger IBS data set (N=109), a smaller sample of age matched Rome III positive IBS patients was chosen to obtain a balanced set of IBS (N=28) and IBD (N=28) patients. Probes showing significant batch effects (38%) were identified and eliminated using BEClear® package in R. Additionally, batch effects were corrected by adding an offset calculated using the group mean differences between healthy controls from the two groups. Differences between methylation profiles of IBS and IBD were tested using ‘limma’ after controlling for the Age variable.

Random forest classification was used to identify probes as described at https://www.analyticsvidhya.com/blog/2016/12/introduction-to-feature-selection-methods-with-an-example-or-how-to-select-the-right-variables/ (Dec. 1, 2016). Briefly, starting from a set of probes/features that were associated with IBD vs IBS (FDR<0.01), we sorted the features based on the variable importance scores for the training data set and tested the accuracy and error rate of the classification using 500 tress, by adding 50 most important features, incrementally. For the selected set of probes with least error rate, we calculated the Area under the Curve (AUC), positive and negative predictive values (PPV and NPV, respectively) using pROC package⁹, and selected cutoffs based on Youden's index¹⁰.

### Results

Twenty eight IBS (67% women, age (average (standard deviation))=25.9 (12.86)) and 28 IBD (46% women, age (average (standard deviation))=22.75 (18.32) years; UC=11, CD=17) were studied. At an FDR<0.01, 3133 CpG sites were different between IBS and IBD.

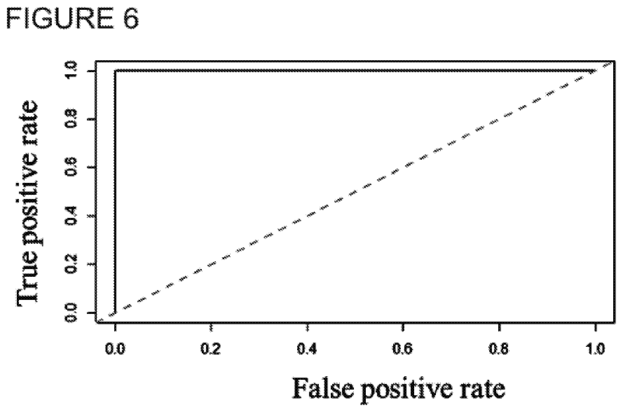

Random forest classification using the methylation profile of 3133 CpG sites identified 100 probes which classified IBS and IBD with least error rate. The overall classification error (out-of-bag (OB) estimate of error rate) was 0% for these probes. FIG. 6 shows the ROC curve to assess the performance of the biomarkers. The area under the ROC curve (AUC) was 1 (p=6.72e-11) for the IBS vs IBD group. As shown in the Table 12, a cutoff of 0.484, resulted in maximum sensitivity 100% for the highest specificity 100% with a PPV of 100% and a NPV of 100%. Table 12 shows the threshold and performance scores for the 100 selected probes that discriminate IBS from IBD. NPV, negative predictive value; PPV, positive predictive value; AUC, area under the curve.

TABLE 12
Performance of DNA-methylation-based markers
in discriminating IBS from IBD.

| Parameter | Value |
|---|---|
| Threshold | 0.484 |
| Sensitivity | 100 |
| Specificity | 100 |
| PPV | 100 |
| NPV | 100 |
| AUC | 1 |
| P value | 6.72e−11 |

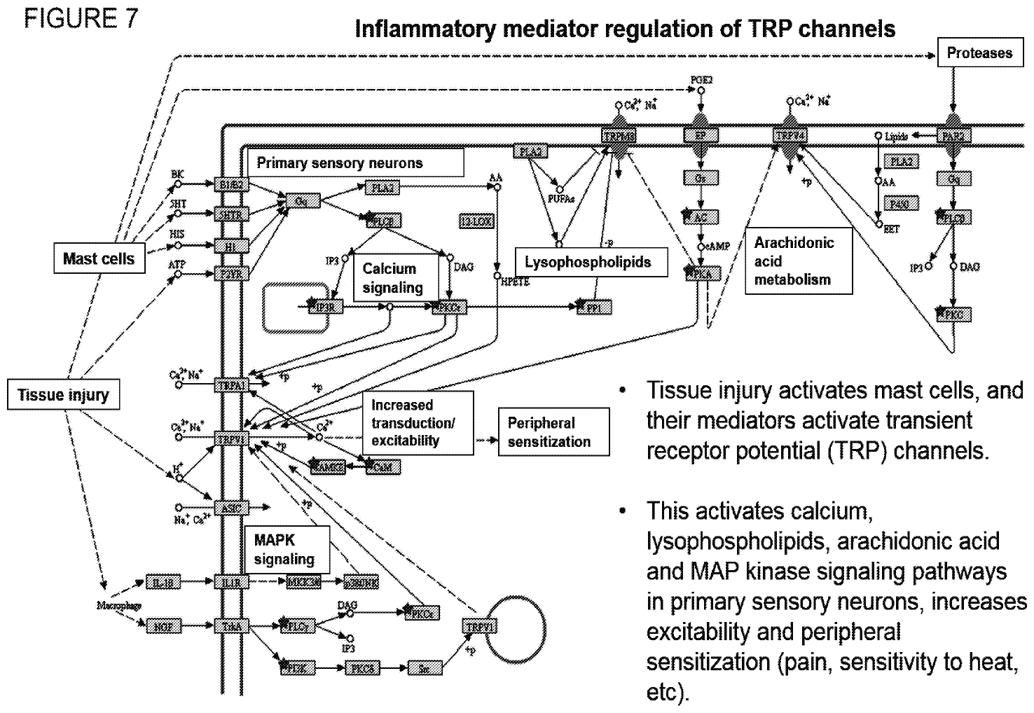

Gene ontology (GO) and pathway analysis of the genes associated with the 3133 genes describes above identified ‘cell junction’ and ‘Inflammatory mediator regulation of TRP channels’ (FDR=1.2% and 13%, respectively) as some of the pathways associated with the gene list. Table 13 shows the differentially methylated genes in IBS vs IBD that were enriched in the ‘inflammatory mediator regulation of the TRP channels’ pathway. Most genes were associated with innate or adaptive immunity. A schematic of the pathway is shown in FIG. 7.

TABLE 13
Differentially methylated genes between IBS and IBD enriched in ‘inflammatory
mediator regulation of TRP channels’ pathway.

| | | | Diff | | Gene | | |
|---|---|---|---|---|---|---|---|
| CpG | p | FDR | (IBS − IBD) | Location | Symbol | Gene name | Gene Function |
| cg03100801 | 5.52E−09 | 5.87E−06 | 0.012713 | Promoter | PLCB4 | phospholipase C, | calcium ion |
| | | | | | | beta 4 | binding |
| cg04162034 | 2.65E−08 | 2.10E−05 | −0.00162 | Promoter | PIK3CD | phosphatidylinositol- | B cell |
| | | | | | | 4,5-bisphosphate 3- | activation/adaptive |
| | | | | | | kinase, catalytic | immune response |
| | | | | | | subunit delta | |
| cg04518808 | 4.17E−07 | 0.000192 | 0.003312 | Promoter | PRKCG | protein kinase C, | neurotrophin TRK |
| | | | | | | gamma | receptor signaling |
| | | | | | | | pathway/regulation |
| | | | | | | | of response to |
| | | | | | | | food |
| cg02073763 | 6.17E−07 | 0.000254 | −0.0025 | Body | PRKCE | protein kinase C, | TRAM-dependent |
| | | | | | | epsilon | toll-like receptor 4 |
| | | | | | | | signaling/innate |
| | | | | | | | immune response |
| cg24523591 | 6.92E−07 | 0.000279 | 0.002529 | Promoter | PIK3CA | phosphatidylinositol- | adaptive immune |
| | | | | | | 4,5-bisphosphate 3- | response/T cell |
| | | | | | | kinase, catalytic | receptor signaling |
| | | | | | | subunit alpha | pathway |
| cg20477591 | 1.47E−06 | 0.000482 | −0.00995 | 3′UTR | PIK3R1 | phosphoinositide-3- | T cell receptor |
| | | | | | | kinase, regulatory | signaling pathway |
| | | | | | | subunit 1 (alpha) | |
| cg09257092 | 1.54E−06 | 0.000496 | 0.001849 | Promoter | ITPR2 | inositol 1,4,5- | calcium ion |
| | | | | | | trisphosphate | transmembrane |
| | | | | | | receptor, type 2 | transporter |
| | | | | | | | activity/innate |
| | | | | | | | immune response |
| cg24904943 | 3.85E−06 | 0.000942 | −0.00784 | Body | ADCY2 | adenylate cyclase 2 | adenylate cyclase |
| | | | | | | | activity/innate |
| | | | | | | | immune response |
| cg12743970 | 1.54E−05 | 0.002268 | 0.001242 | Body | PRKCA | protein kinase C, | activation of |
| | | | | | | alpha | adenylate cyclase |
| | | | | | | | activity/innate |
| | | | | | | | immune response |
| cg07397481 | 2.17E−05 | 0.002781 | 0.008682 | Body | PLCG2 | phospholipase C, | innate immune |
| | | | | | | gamma 2 | response/T cell |
| | | | | | | (phosphatidylinositol- | receptor signaling |
| | | | | | | specific) | pathway |
| cg06153873 | 2.94E−05 | 0.003379 | −0.00083 | Body | ADCY9 | adenylate cyclase 9 | adenylate cyclase |
| | | | | | | | activity/innate |
| | | | | | | | immune response |
| cg01776691 | 4.39E−05 | 0.004303 | 0.003935 | Body | CAMK2B | calcium/calmoduiin- | interferon-gamma- |
| | | | | | | dependent protein | mediated signaling |
| | | | | | | kinase II beta | pathway |
| cg01937808 | 8.30E−05 | 0.006212 | 0.017939 | Body | ADCY9 | adenylate cyclase 9 | adenylate cyclase |
| | | | | | | | activity/innate |
| | | | | | | | immune response |
| cg01661235 | 0.000103 | 0.007089 | 0.003343 | Body | PRKCB | protein kinase C, | positive regulation |
| | | | | | | beta | of NF-kappaB |
| | | | | | | | transcription factor |
| cg12245996 | 0.000108 | 0.007305 | −0.00279 | Promoter | PRKCQ | protein kinase C, | positive regulation |
| | | | | | | theta | of interleukin-4 |
| | | | | | | | production |
| cg17119568 | 0.000113 | 0.007488 | −0.00065 | Promoter | PRKACA | protein kinase, | innate immune |
| | | | | | | cAMP-dependent, | response |
| | | | | | | catalytic, alpha | |
| cg17244462 | 0.000133 | 0.008218 | −0.03554 | Promoter | CAMK2D | calcium/calmodulin- | ion channel |
| | | | | | | dependent protein | binding |
| | | | | | | kinase II delta | |
| cg01579928 | 0.000152 | 0.008841 | 0.001593 | Promoter | CALM1 | calmodulin 1 | G-protein coupled |
| | | | | | | (phosphorylase | receptor signaling |
| | | | | | | kinase, delta) | pathway |
| cg09793776 | 0.000166 | 0.009283 | 0.001351 | Body | PPP1CA | protein phosphatase | transforming |
| | | | | | | 1, catalytic subunit, | growth factor beta |
| | | | | | | alpha isozyme | receptor signaling |
| | | | | | | | pathway |

Legend: FDR, false detection rate; MeanDiff, difference between mean beta values of IBS and IBD. Methylation-Hyper, hypermethylated in IBS compared to IBD; Hypo, hypomethylated in IBS compared to IBD.

### Discussion

We identified a set of DNA methylation-based biomarkers in PBMCs that discriminated IBS from IBD. An important difference between IBS and IBD is the presence of tissue damage and inflammation in IBD which is absent in IBS. Gene ontology terms associated with the differentially methylated genes, such as those related to inflammation support the importance and ability of these probes in differentiating the diseases. In particular the association of inflammatory mediator regulation of TRP channels was interesting, since the TRP channels can be modulated indirectly by inflammatory mediators such as PGE2, bradykinin, ATP, NGF, and proinflammatory cytokines that are generated during tissue injury. While the noxious heat receptor TRPV1 is sensitized (that is, their excitability can be increased) by post-translational modifications upon activation of G-protein coupled receptors (GPCRs) or tyrosine kinase receptors, the receptors for inflammatory mediators, the same action appears to mainly desensitize TRPM8, the main somatic innocuous cold sensor¹¹. This sensitization could allow the receptor to become active at body temperature, so it not only contributes toward thermal hypersensitivity but also is possibly a substrate for ongoing persistent pain¹².

Although extensive correction for technical and known variation such as batch effect and age was performed, some of the observed differences could potentially be attributed to the systematic differences in patient population. IBD group was mostly pediatric (21% adults). In addition, the methylation profiling was performed on peripheral blood leukocytes which included PBMCs as well as granulocytes compared to just PBMCs that were used for the study involving IBS and controls. Given these limitations, the suggested biomarkers warrant further validation in the PBMCs of adult IBD patients, and other GI disease controls including celiac disease and colon cancer.

### REFERENCES

1. Longstreth G F, et al. Gastroenterology 2006; 130:1480-1491.
2. Hoekman D R, et al. Eur J Gastroenterol Hepatol 2017:29:1086-1090.
3. Makharia A, et al. Nutrients 2015; 7:10417-10426.
4. Camilleri M, et al. Expert Rev Gastroenterol Hepatol 2017; 11:303-316.
5. Kim H, et al. J Genet Genomics Yi Chuan Xue Bao 2018; 45:87-97.
6. Suv\dot{a} M L, et al. Science 2013; 339:1567-1570.
7. Jenuwein T, Allis C D. Science 2001; 293:1074-1080.
8. Akulenko R, et al. PloS One 2016; 11:e0159921.
9. Robin X, et al. BMC Bioinformatics 2011; 12:77.
10. Youden W J. Cancer 1950; 3:32-35.
11. Dhaka A, et al. Annu Rev Neurosci 2006; 29:135-161.
12. Levine J D, Alessandri-Haber N. Biochim Biophys Acta 2007; 1772:989-1003.

### Example 3: DNA Methylation Based Biomarkers for Discriminating IBS from Healthy Controls

This Example demonstrates a further analysis of IBS methylation profiles.

K-Fold Cross Validation of 550 Biomarkers of IBS Vs Healthy Controls (HCs)

We performed repeated K-fold cross validation with 90% training and 10% test split and 10 repeats. The final sensitivity and specificity, which are means of sensitivity and specificity for all the repeats, was 81% and 91%, respectively, which was comparable or slightly better than our previous validation. To further ensure that there was no model overfitting, we randomly shuffled the disease status labels of the subjects and reran the K-fold cross validation. As expected the sensitivity and specificity of the shuffled labels was very low (51% and 60%) suggesting that the markers are specifically discriminate IBS vs. HCs.

Selection of a Smaller Set of Biomarkers Discriminating IBS Vs HCs Using WGCNA

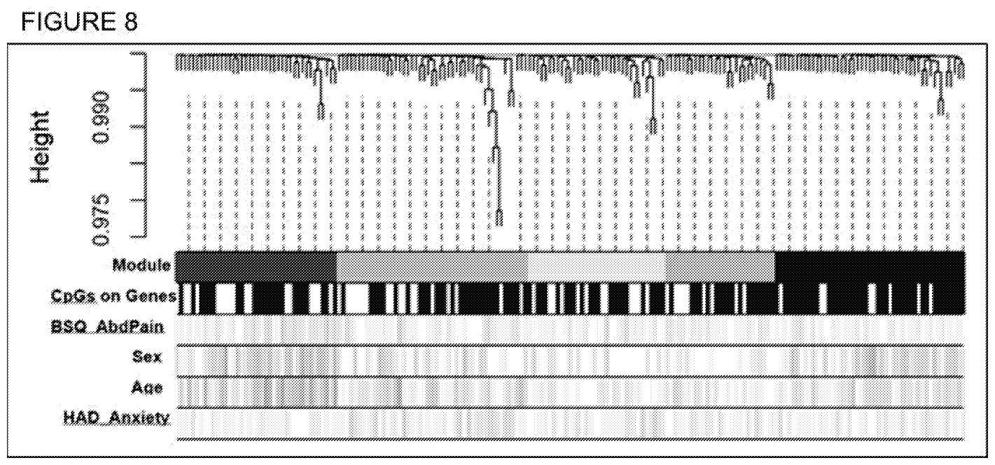

We firstly used the 550 biomarkers to construct a weighted gene co-expression network (WGCNA)¹, and then to identify gene modules with markers that were highly correlated (co-methylated) and associated with clinical features of IBS. Main aim of this analysis was to identify a smaller set of markers² that can discriminate IBS from HCs. We identified 5 modules with markers that were highly correlated at the methylation level but also with other phenotypes such as abdominal pain, Age, Sex and hospital anxiety depression (HAD) score (FIG. 8, Table 14). Majority of the markers were present on the gene (represented by black lines on the 1^{st} bar in FIG. 8) compared to the ones outside the gene (represented by black lines on the 1st bar in FIG. 8).

There was a statistically significant association of ‘Brown’ module with Age, Sex and Abdominal pain. ‘Blue’ module was associated with Age and Sex (p<0.05). Table 14 shows the bicorrelation of modules between methylation and clinical traits.

TABLE 14
Bicorrelation between methylation modules and traits in IBS.

| | Abdominal | Abdominal | | | | | Anxiety | Anxiety |
|---|---|---|---|---|---|---|---|---|
| | pain bicor | pain p | Sex r | Sex p | Age bicor | Age p | bicor | p |
| MEblue | 0.003 | 0.975 | 0.335 | 0.0005 | −0.256 | 0.007 | −0.016 | 0.873 |
| MEbrown | 0.196 | 0.042 | 0.345 | 0.002 | 0.415 | 0.0001 | 0.013 | 0.897 |
| MEgreen | −0.027 | 0.785 | 0.000 | 0.687 | 0.053 | 0.587 | 0.067 | 0.493 |
| MEgrey | 0.023 | 0.811 | 0.018 | 0.173 | 0.077 | 0.424 | −0.009 | 0.928 |
| MEturquoise | 0.067 | 0.493 | 0.0001 | 0.318 | −0.032 | 0.744 | 0.163 | 0.092 |
| MEyellow | −0.065 | 0.501 | 0.0001 | 0.494 | −0.062 | 0.521 | 0.104 | 0.282 |

Ten biomarkers that were highly correlated were chosen from each module. There were over 355 markers whose expression was not correlated with others, leading to a total of 405 unique non-overlapping markers, which was tested for accuracy of prediction of IBS status.

The training data set consisted of 54 samples, which was trained using a 90:10 split of training: test data and sampled 10 times. It was again tested on unseen samples (N=22). Training and testing using K-fold validation gave a sensitivity and specificity of 92% and 90% respectively, using the 405 biomarkers (Table 15).

TABLE 15
Sensitivity and specificity of the selected biomarkers.

| Parameter | Value |
|---|---|
| Sensitivity | 0.92 |
| Specificity | 0.90 |
| Pos Pred Value | 0.92 |
| Neg Pred Value | 0.90 |
| P value | 0.0002906 |
| Accuracy (95% CI) | 0.91 (0.7084, 0.9888) |

The entire list of 405 markers is given in the Table 16.

### REFERENCES

1. Langfelder P, Horvath S. BMC Bioinformatics. 2008 Dec. 29; 9:559. PMCID: PMC2631488
2. Yuan L, et al. Front Genet [Internet]. 2018 Aug. 15 [cited 2019 May 28]; 9. Available from: https://www.ncbi.nlm.nih.gov/pmc/articles/PMC6104177/PMCID: PMC6104177

### Example 4: Analysis of DNA Methylation Based Biomarkers in PBMCS for IBD Vs HCs

We identified 200 differentially methylated CpG sites between IBD (N=28, UC=11, CD=17) and HCs (N=20) from the published data set1. We calculated the relative importance score for differentially methylated sites using random forest classification2, separately for ulcerative colitis (UC) and Crohn's disease (CD). We ordered the markers by their importance scores. We chose the top 50 probes and incrementally tested optimal number of probes that resulted in minimum classification error. To validate the probes, a 10 fold cross validation was repeated 10 times and average sensitivity and specificity was recorded. We identified 50 markers that discriminated UC from healthy controls (97% accuracy) and 50 markers that discriminated CD from healthy controls (100% accuracy).

The full list of UC and CD biomarkers is presented in Tables 17 and 18). Gene ontology (GO) terms associated with the biomarkers of UC included “oxidation-reduction process”. GO terms associated with CD biomarkers included “metal ion binging”.

Therefore, the IBD disease vs. control panel included 200 non-overlapping set of markers that discriminated IBS from IBD (N=100), UC from healthy controls (N=50) and CD from healthy controls (N=50).

### REFERENCES

1. Harris R A, et al. Inflamm Bowel Dis. 2012 December; 18(12):2334-2341. PMCID: PMC3812910
2. Breiman L. Random forests. Mach Learn. 2001; 45(1):5-32.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

TABLE 16

| | | | | Relation | UCSC Ref | UCSC Ref Gene |
|---|---|---|---|---|---|---|
| ID | chr | pos | strand | to Island | Gene Name | Accession |
| cg00067600 | 3 | 34109141 | − | OpenSea | | |
| cg00157199 | 20 | 29551622 | + | Island | | |
| cg00181968 | 1 | 179712204 | + | Island | FAM163A | NM_173509 |
| cg00223950 | 1 | 19536222 | + | Island | UBR4 | NM_020765 |
| cg00242358 | 3 | 47619041 | − | N_Shore | CSPG5 | NM_006574 |
| cg00293191 | 16 | 73126120 | − | OpenSea | HTA | NR_027756 |
| cg00367396 | 6 | 42694557 | − | N_Shore | | |
| cg00467202 | 16 | 30759573 | + | Island | PHKG2 | NM_000294 |
| cg00519002 | 16 | 56659338 | − | Island | MT1E | NM_175617 |
| cg00632914 | 5 | 178359659 | + | OpenSea | ZFP2 | NM_030613 |
| cg00649515 | 1 | 44456953 | − | N_Shore | CCDC24 | NM_152499 |
| cg00656860 | 15 | 68116607 | + | Island | LBXCOR1 | NM_001031807 |
| cg00660106 | 10 | 118885753 | − | Island | | |
| cg00760321 | 1 | 32671490 | + | Island | IQCC; | NM_018134; |
| | | | | | IQCC | NM_001160042 |
| cg00807237 | 7 | 934156 | − | N_Shore | C7orf20 | NM_015949 |
| cg00859655 | 11 | 7694524 | + | N_Shore | CYB5R2 | NM_016229 |
| cg01029238 | 15 | 76136129 | − | Island | UBE2Q2; | NM_173469; |
| | | | | | UBE2Q2 | NM_001145335 |
| cg01048272 | 17 | 80169878 | − | N_Shore | CCDC57 | NM_198082 |
| cg01050736 | 6 | 32710583 | − | OpenSea | HLA-DQA2 | NM_020056 |
| cg01184449 | 2 | 42396170 | + | N_Shore | EML4; | NM_019063; |
| | | | | | EML4 | NM_001145076 |
| cg01225095 | 12 | 32909323 | + | S_Shore | YARS2 | NM_001040436 |
| cg01395533 | 3 | 66024640 | + | Island | MAGI1; | NM_004742; |
| | | | | | MAGI1; | NM_015520; |
| | | | | | MAGI1 | NM_001033057 |
| cg01399317 | 8 | 144810339 | − | Island | FAM83H | NM_198488 |
| cg01405445 | 19 | 36246406 | − | Island | HSPB6 | NM_144617 |
| cg01437917 | 17 | 4688569 | + | N_Shore | | |
| cg01456989 | 11 | 61897937 | + | OpenSea | INCENP; | NM_020238; |
| | | | | | INCENP | NM_001040694 |
| cg01524860 | 6 | 99290284 | + | Island | | |
| cg01673669 | 15 | 60298900 | + | S_Shore | | |
| cg01678799 | 15 | 43481741 | + | S_Shelf | CCNDBP1; | NM_012142; |
| | | | | | CCNDBP1; | NR_027513; |
| | | | | | CCNDBP1; | NR_027514; |
| | | | | | CCNDBP1 | NM_037370 |
| cg01763636 | 12 | 132568565 | − | N_Shore | EP400NL | NR_003290 |
| cg01837362 | 12 | 34492938 | − | N_Shore | | |
| cg01915076 | 12 | 49393228 | + | Island | DDN | NM_015086 |
| cg01917669 | 21 | 47056698 | + | N_Shore | | |
| cg01947695 | 16 | 34378069 | + | OpenSea | | |
| cg02141498 | 16 | 86912611 | − | Island | | |
| cg02156314 | 10 | 31607685 | − | Island | ZEB1; | NR_024286; |
| | | | | | ZEB1; | NM_030751; |
| | | | | | ZEB1; | NR_024287; |
| | | | | | ZEB1; | NR_024285; |
| | | | | | LOC220930 | NR_024284 |
| cg02159111 | 2 | 239361488 | − | N_Shore | | |
| cg02229605 | 14 | 61119122 | + | S_Shelf | | |
| cg02247356 | 6 | 150390274 | − | Island | ULBP3 | NM_024518 |
| cg02253142 | 15 | 52048211 | + | S_Shelf | TMOD2; | NM_014548; |
| | | | | | TMOD2 | NM_001142885 |
| cg02259284 | 3 | 105085701 | + | N_Shore | ALCAM | NM_001627 |
| cg02305687 | 5 | 33937182 | − | Island | RXFP3 | NM_016568 |
| cg02336461 | 17 | 57697246 | − | Island | CLTC; | NM_004859; |
| | | | | | CLTC | NM_004859 |
| cg02386875 | 17 | 28927378 | + | Island | LRRC37B2 | NR. 015341 |
| cg02451502 | 13 | 22486732 | + | OpenSea | | |
| cg02601685 | 17 | 55055297 | − | Island | SCPEP1 | NM_021626 |
| cg02619599 | 5 | 1385632 | + | N_Shore | | |
| cg02627599 | 6 | 148880600 | + | N_Shore | | |
| cg02704931 | 15 | 57998412 | − | N_Shore | GCOM1; | NM_001018090; |
| | | | | | GRINL1A; | NM_015532; |
| | | | | | GRINL1A; | NM_001018102; |
| | | | | | GCOM1; | NM_001018091; |
| | | | | | GRINL1A | NR_027390 |
| cg02792794 | 12 | 109592449 | − | Island | ACACB | NM_001093 |
| cg02797036 | 7 | 44084886 | − | S_Shore | DBNL; | NM_001122956; |
| | | | | | DBNL; | NM_001014436; |
| | | | | | DBNL | NM_014063 |
| cg02847037 | 13 | 22033729 | + | Island | ZDHHC20 | NM_153251 |
| cg02859655 | 19 | 3648642 | − | Island | PIP5K1C | NM_012398 |
| cg02930078 | 6 | 17706856 | + | Island | NUP153 | NM_005124 |
| cg02953397 | 1 | 1238678 | + | N_Shelf | ACAP3 | NM_030649 |
| cg03051116 | 10 | 30720703 | − | N_Shore | | |
| cg03163246 | 4 | 74921023 | + | OpenSea | PPBPL2 | NR_026769 |
| cg03296248 | 13 | 110918516 | + | Island | COL4A1 | NM_001845 |
| cg03420242 | 16 | 49893122 | − | Island | | |
| cg03422070 | 11 | 2722082 | + | Island | KCNQ1OT1; | NR_002728; |
| | | | | | KCNQ1; | NM_000218; |
| | | | | | KCNQ1 | NM_181798 |
| cg03440762 | 5 | 174158195 | + | N_Shore | | |
| cg03521085 | 4 | 1219008 | + | Island | CTBP1; | NM_001328; |
| | | | | | CTBP1 | NM_001012614 |
| cg03565674 | 11 | 1507321 | − | OpenSea | HCCA2 | NM_053005 |
| cg03585974 | 20 | 2800511 | + | N_Shore | | |
| cg03622371 | 11 | 73000436 | − | OpenSea | P2RY6; | NM_176797; |
| | | | | | P2RY6; | NM_176796; |
| | | | | | P2RY6; | NM_004154; |
| | | | | | P2RY6 | NM_176798 |
| cg03742273 | 1 | 2772664 | + | Island | | |
| cg03830093 | 17 | 8731697 | − | OpenSea | PIK3R6 | NM_001010855 |
| cg03909886 | 10 | 42606808 | − | Island | | |
| cg03933810 | 17 | 4634545 | − | N_Shore | MED11 | NM_001001683 |
| cg04001333 | 14 | 76045348 | + | S_Shore | FLVCR2 | NM_017791 |
| cg04049508 | 16 | 55535988 | − | OpenSea | MMP2; | NM_001127891; |
| | | | | | MMP2 | NM_004530 |
| cg04080595 | 1 | 2985649 | + | Island | PRDM16; | NM_022114; |
| | | | | | FLJ42875; | NR_015440; |
| | | | | | PRDM16; | NM_199454; |
| | | | | | FLJ42875 | NR_024371 |
| cg04094193 | 4 | 69216103 | − | S_Shore | YTHDC1; | NM_133370; |
| | | | | | YTHDC1 | NM_001031732 |
| cg04162647 | 14 | 99734598 | − | N_Shore | BCL11B; | NM_022898; |
| | | | | | BCL11B | NM_138576 |
| cg04177091 | 16 | 1030166 | + | Island | | |
| cg04267807 | 7 | 83278462 | + | OpenSea | SEMA3E | NM_012431 |
| cg04335449 | 3 | 127842704 | − | Island | RUVBL1 | NM_003707 |
| cg04400533 | 3 | 185788716 | + | OpenSea | ETV5 | NM_004454 |
| cg04430996 | 16 | 591437 | − | Island | SOLH | NM_005632 |
| cg04433035 | 17 | 36105064 | + | N_Shore | HNF1B; | NM_001165923; |
| | | | | | HNF1B; | NM_000458; |
| | | | | | HNF1B; | NM_001165923; |
| | | | | | HNF1B | NM_000458 |
| cg04444415 | 3 | 193272869 | + | OpenSea | ATP13A4 | NM_032279 |
| cg04893513 | 16 | 902736 | − | N_Shore | | |
| cg05019671 | 5 | 77943786 | + | N_Shore | LHFPL2 | NM_005779 |
| cg05116255 | 10 | 37413782 | − | N_Shore | ANKRD30A | NM_052997 |
| cg05158074 | 12 | 111284585 | − | OpenSea | CCDC63 | NM_152591 |
| cg052l3661 | 5 | 1227968 | + | S_Shore | SLC6A18 | NM_182632 |
| cg05232576 | 20 | 2777921 | − | N_Shelf | CPXM1 | NM_019609 |
| cg05241015 | 2 | 1748876 | + | Island | PXDN | NM_012293 |
| cg05263760 | 6 | 12845743 | − | OpenSea | PHACTR1 | NM_030948 |
| cg05384646 | 15 | 30206862 | + | OpenSea | | |
| cg05431257 | 2 | 20370211 | − | Island | GDF7 | NM_182828 |
| cg05498024 | 10 | 28917278 | − | OpenSea | | |
| cg05583103 | 19 | 36157607 | − | OpenSea | UPK1A | NM_007000 |
| cg05592911 | 11 | 68722879 | − | OpenSea | | |
| cg05627987 | 19 | 51220286 | + | Island | SHANK1 | NM_016148 |
| cg05872236 | 18 | 47825225 | + | Island | | |
| cg06018240 | 7 | 150768388 | − | OpenSea | SLC4A2 | NM_003040 |
| cg06046805 | 5 | 58299024 | − | OpenSea | PDE4D; | NM_001165899; |
| | | | | | PDE4D; | NM_001104631; |
| | | | | | PDE4D | NM_006203 |
| cg06138505 | 3 | 127317209 | + | Island | MCM2 | NM_004526 |
| cg06225979 | 18 | 32957337 | − | Island | ZNF396 | NM_145756 |
| cg06287140 | 12 | 122468180 | + | OpenSea | BCL7A; | NM_001024808; |
| | | | | | BCL7A | NM_020993 |
| cg06289444 | 6 | 169631196 | + | N_Shore | THBS2 | NM_003247 |
| cg06297356 | 16 | 1012735 | + | S_Shore | LMF1 | NM_022773 |
| cg06395280 | 2 | 242295608 | − | Island | FARP2 | NM_014808 |
| cg06419850 | 17 | 4688683 | + | Island | VMO1; | NM_001144941; |
| | | | | | VMO1; | NM_001144940; |
| | | | | | VMO1; | NM_001144939; |
| | | | | | VMO1 | NM_182566 |
| cg06466839 | 11 | 129062277 | − | OpenSea | RIGS | NM. 001142685 |
| cg06514855 | 17 | 21220141 | + | Island | | |
| cg06525670 | 13 | 30528708 | + | OpenSea | | |
| cg06704589 | 13 | 113097900 | + | OpenSea | | |
| cg06745507 | 15 | 44085196 | − | Island | SERF2 | NM_001018108 |
| cg06765785 | 11 | 2020391 | + | S_Shore | H19 | NR_002196 |
| cg06938878 | 11 | 15094364 | − | N_Shore | CALCB | NM_000728 |
| cg06940110 | 2 | 118616430 | − | N_Shore | | |
| cg06944693 | 11 | 130299298 | + | S_Shore | ADAMTS8 | NM_007037 |
| cg06980252 | 7 | 137605253 | + | OpenSea | CREB3L2 | NM_194071 |
| cg07004386 | 14 | 70040391 | − | N_Shore | | |
| cg07327178 | 5 | 8457729 | + | Island | | |
| cg07337446 | 6 | 46517898 | + | OpenSea | CYP39A1 | NM_016593 |
| cg07464206 | 11 | 15960015 | − | Island | | |
| cg07470512 | 20 | 32255052 | − | Island | NECAB3; | NM_031231; |
| | | | | | NECAB3; | NM_031232; |
| | | | | | C20orf134; | NM_001024675; |
| | | | | | C20orf134 | NM_001024675 |
| cg07592012 | 17 | 79977307 | + | N_Shelf | STRA13 | NM_144998 |
| cg07632934 | 15 | 25422945 | − | OpenSea | SNORD115-9; | NR_003301; |
| | | | | | SNORD115-5; | NR_003297; |
| | | | | | SNORD115-12; | NR_003304; |
| | | | | | SNORD115-10 | NR_003302 |
| cg07657332 | 10 | 54643873 | + | OpenSea | | |
| cg07664272 | 4 | 138774829 | + | OpenSea | | |
| cg07665718 | 19 | 16244068 | − | OpenSea | HSH2D; | NM_032855; |
| | | | | | RAB8A | NM_005370 |
| cg07679834 | 19 | 19841125 | + | N_Shelf | ZNF14 | NM_021030 |
| cg07695826 | 18 | 51884330 | − | Island | C18orf54 | NM_173529 |
| cg07802917 | 3 | 123603311 | + | Island | MYLK; | NM_053025; |
| | | | | | MYLK; | NM_053026; |
| | | | | | MYLK; | NM_053028; |
| | | | | | MYLK | NM_053027 |
| cg08031958 | 21 | 47009726 | − | N_Shore | | |
| cg08134324 | 16 | 342012 | − | S_Shore | AXIN1; | NM_003502; |
| | | | | | AXIN1 | NM_181050 |
| cg08162465 | 1 | 87668671 | − | OpenSea | | |
| cg08211853 | 2 | 3658933 | + | OpenSea | COLEC11; | NM_024027; |
| | | | | | COLEC11 | NM_199235 |
| cg08220120 | 5 | 138727711 | − | Island | LOC389333; | NM_001161546; |
| | | | | | LOC389333 | NM_001161546 |
| cg08440920 | 16 | 54963401 | − | Island | | |
| cg08475853 | 4 | 9492758 | + | N_Shelf | | |
| cg08494157 | 16 | 1429719 | + | Island | UNKL | NM_023076 |
| cg08569350 | 7 | 1069812 | + | S_Shore | C7orf50; | NM_001134395; |
| | | | | | C7orf50; | NM_032350; |
| | | | | | C7orf50 | NM_001134396 |
| cg08627624 | 10 | 70031114 | − | OpenSea | | |
| cg08686879 | 5 | 131409634 | + | OpenSea | CSF2 | NM_000758 |
| cg08704196 | 16 | 34787392 | − | Island | | |
| cg08791849 | 13 | 42846648 | − | Island | AKAP11 | NM_016248 |
| cg08395260 | 11 | 82756563 | − | OpenSea | RAB30 | NM_014488 |
| cg08909157 | 9 | 215561 | − | S_Shore | DOCK8; | NM_203447; |
| | | | | | C9orf66; | NM_152569; |
| | | | | | C9orf66 | NM_152569 |
| cg08994789 | 17 | 28903642 | − | Island | LRRC37B2 | NR_015341 |
| cg09100014 | 16 | 85937496 | + | OpenSea | IRF8 | NM_002163 |
| cg09235723 | 11 | 65495317 | − | OpenSea | | |
| cg09266113 | 1 | 54356345 | − | S_Shore | YIPF1 | NM_018982 |
| cg09290175 | 22 | 39688265 | + | Island | | |
| cg09298014 | 14 | 97058864 | + | N_Shore | | |
| cg09330551 | 19 | 51334432 | + | S_Shelf | KLK15; | NM_017509; |
| | | | | | KLK15; | NM_138563; |
| | | | | | KLK15 | NM_138564 |
| cg09379188 | 8 | 59036394 | − | OpenSea | FAM110B | NM_147189 |
| cg09423312 | 7 | 1163549 | + | OpenSea | C7orf50; | NM_001134395; |
| | | | | | C7orf50; | NM_032350; |
| | | | | | C7orf50 | NM_001134396 |
| cg09440812 | 11 | 66610597 | − | Island | RCE1; | NM_005133; |
| | | | | | RCE1; | NM_001032279; |
| | | | | | C11orf80 | NM_024650 |
| cg09618694 | 3 | 56591190 | − | Island | CCDC66; | NM_001012506; |
| | | | | | CCDC66; | NM_001141947; |
| | | | | | CCDC66; | NR_024460; |
| | | | | | CCDC66; | NM_001141947; |
| | | | | | CCDC66 | NM_001012506 |
| cg09622957 | 1 | 150521190 | − | N_Shore | ADAMTSL4; | NM_019032; |
| | | | | | ADAMTSL4 | NM_025008 |
| cg09894655 | 2 | 132430151 | − | N_Shore | | |
| cg09928766 | 19 | 51815373 | − | Island | IGLON5 | NM_001101372 |
| cg09955084 | 16 | 413813 | + | S_Shelf | | |
| cg09962377 | 16 | 522007 | − | OpenSea | RAB11FIP3 | NM_014700 |
| cg10052164 | 11 | 1750939 | − | OpenSea | HCCA2 | NM_053005 |
| cg10081621 | 15 | 94808566 | + | OpenSea | | |
| cg10113910 | 16 | 34579002 | + | OpenSea | | |
| cg10140536 | 19 | 57630596 | + | Island | USP29 | NM_020903 |
| cg10194829 | 8 | 21900177 | + | OpenSea | FGF17 | NM_003867 |
| cg10212537 | 14 | 93799559 | − | Island | BTBD7; | NM_018167; |
| | | | | | BTBD7; | NM_001002860; |
| | | | | | KIAA1409 | NM_020818 |
| cg10290200 | 7 | 128481295 | − | N_Shore | FLNC; | NM_001127487; |
| | | | | | FLNC | NM_001458 |
| cg10290276 | 11 | 2291754 | − | Island | ASCL2; | NM_005170; |
| | | | | | ASCL2 | NM_005170 |
| cg10318443 | 17 | 685449 | + | Island | GLOD4; | NM_016080; |
| | | | | | RNMTL1 | NM_018146 |
| cg10334121 | 3 | 137728995 | − | Island | CLDN18; | NM_016369; |
| | | | | | CLDN18 | NM_001002026 |
| cg10436026 | 13 | 37453429 | − | OpenSea | SMAD9; | NM_001127217; |
| | | | | | SMAD9 | NM_005905 |
| cg10460130 | 2 | 242625978 | − | Island | DTYMK; | NM_001165031; |
| | | | | | DTYMK; | NM_012145; |
| | | | | | DTYMK | NR_033255 |
| cg10536462 | 4 | 870491 | − | N_Shore | GAK | NM_005255 |
| cg10682833 | 19 | 49614538 | + | N_Shelf | | |
| cg10791023 | 3 | 122005090 | − | OpenSea | CASR | NM_000388 |
| cg10915739 | 21 | 34405733 | + | Island | | |
| cg10984252 | 10 | 115439257 | − | Island | CASP7; | NM_033340; |
| | | | | | CASP7; | NM_033338; |
| | | | | | CASP7; | NM_001227; |
| | | | | | CASP7 | NM_033339 |
| cg10985158 | 1 | 201689688 | − | OpenSea | NAV1 | NM_020443 |
| cg11079278 | 13 | 46425210 | − | OpenSea | SIAH3 | NM_198849 |
| cg11165881 | 3 | 238048 | + | N_Shore | CHL1 | NM_006614 |
| cg11461030 | 1 | 46932239 | + | Island | | |
| cg11524248 | 5 | 149545907 | + | N_Shore | CDX1 | NM_001804 |
| cg11558474 | 9 | 74382743 | + | N_Shore | TMEM2; | NM_013390; |
| | | | | | TMEM2 | NM_001135820 |
| cg11591485 | 3 | 124839640 | + | OpenSea | SLC12A8 | NM_024628 |
| cg11628781 | 5 | 1089021 | + | Island | SLC12A7 | NM_006598 |
| cg11706080 | 2 | 12856970 | − | Island | TRIB2; | NM_021643; |
| | | | | | TRIB2 | NR_027303 |
| cg11710912 | 13 | 107217605 | − | N_Shelf | ARGLU1 | NM_018011 |
| Cfl11835619 | 12 | 56475064 | − | S_Shore | ERBB3; | NM_001005915; |
| | | | | | ERBB3 | NM_001982 |
| cg11902379 | 19 | 7681598 | − | N_Shore | KIAA1543; | NM_020902; |
| | | | | | KIAA1543 | NM_001080429 |
| cg11915641 | 2 | 103236553 | + | Island | SLC9A2 | NM_003048 |
| cg12039242 | 8 | 101962991 | + | Island | YWHAZ; | NM_001135701; |
| | | | | | YWHAZ; | NM_003406; |
| | | | | | YWHAZ; | NM_145690; |
| | | | | | YWHAZ; | NM_001135700; |
| | | | | | YWHAZ; | NM_001135699; |
| | | | | | YWHAZ | NM_001135702 |
| cg12039422 | 17 | 12325031 | + | OpenSea | | |
| cg12118784 | 19 | 47141551 | − | N_Shore | | |
| cg12129897 | 11 | 62559856 | + | S_Shore | NXF1; | NM_001081491; |
| | | | | | NXF1; | NM_006362; |
| | | | | | TMEM223 | NM_001080501 |
| cg12304381 | 7 | 57710785 | + | OpenSea | | |
| cg12346874 | 2 | 137523644 | − | Island | | |
| cg12373573 | 12 | 85430025 | − | OpenSea | TSPAN19; | NM_001100917; |
| | | | | | TSPAN19; | NM_001100917; |
| | | | | | LRRIQ1; | NM_001079910; |
| | | | | | LRRIQ1 | NM_032165 |
| cg12428906 | 11 | 18034757 | − | Island | SERGEF | NM_012139 |
| cg12451679 | 19 | 47617189 | − | S_Shore | ZC3H4 | NM_015168 |
| cg12463883 | 13 | 108237965 | − | OpenSea | FAM155A | NM_001080396 |
| cg12531953 | 8 | 56793075 | + | Island | LYN; | NM_002350; |
| | | | | | LYN | NM_001111097 |
| cg12594244 | 15 | 74218754 | + | Island | LOXL1 | NM_005576 |
| cg12712184 | 7 | 155790801 | − | OpenSea | | |
| cg12796916 | 11 | 67471698 | − | OpenSea | | |
| cg12798700 | 6 | 125622988 | + | Island | HDDC2 | NM_016063 |
| cg12851717 | 13 | 100517009 | − | OpenSea | CLYBL | NM_206808 |
| cg12905865 | 1 | 6545814 | + | S_Shore | PLEKHG5; | NM_020631; |
| | | | | | PLEKHG5; | NM_001042665; |
| | | | | | PLEKHG5; | NM_198681; |
| | | | | | PLEKHG5; | NM_001042664; |
| | | | | | PLEKHG5 | NM_001042663 |
| cg12922648 | 6 | 30877739 | + | S_Shore | GTF2H4 | NM_001517 |
| cg13092108 | 1 | 26857284 | − | S_Shore | RPS6KA1 | NM_002953 |
| cg13341153 | 10 | 120001043 | + | OpenSea | | |
| cg13357482 | 6 | 105628017 | + | S_Shore | POPDC3 | NM_022361 |
| cg13372743 | 8 | 52322134 | − | Island | PXDNL | NM_144651 |
| cg13627062 | 15 | 96888433 | − | N_Shore | | |
| cg13629887 | 12 | 12876682 | + | Island | | |
| cg13702942 | 5 | 178594374 | + | Island | ADAMTS2; | NM_021599; |
| | | | | | ADAMTS2 | NM_014244 |
| cg13707224 | 9 | 1043825 | − | N_Shore | | |
| cg13782176 | 10 | 6264761 | − | S_Shore | PFKFB3; | NM_004566; |
| | | | | | PFKFB3 | NM_001145443 |
| cg13788515 | 2 | 207139445 | − | Island | ZDBF2 | NM_020923 |
| cg13900773 | 12 | 130824015 | − | Island | PIWIL1 | NM_004764 |
| cg14016554 | 6 | 31696509 | + | Island | DDAH2 | NM_013974 |
| cg14025831 | 20 | 3873404 | + | S_Shelf | PANK2; | NM_153638; |
| | | | | | PANK2; | NM_024960; |
| | | | | | PANK2 | NM_153640 |
| cg14074117 | 16 | 1909714 | + | OpenSea | C16orf73; | NM_152764; |
| | | | | | C16orf73 | NM_001163560 |
| cg14076923 | 9 | 124081083 | + | OpenSea | GSN; | NM_198252; |
| | | | | | GSN; | NM_001127665; |
| | | | | | GSN; | NM_001127667; |
| | | | | | GSN; | NM_001127664; |
| | | | | | GSN; | NM_001127663; |
| | | | | | GSN; | NM_000177; |
| | | | | | GSN; | NM_001127666; |
| | | | | | GSN | NM_001127662 |
| cg14154547 | 9 | 137293309 | − | OpenSea | RXRA | NM_002957 |
| cg14162912 | 2 | 238600607 | − | Island | LRRFIP1; | NM_001137550; |
| | | | | | LRRFIP1; | NM_001137552; |
| | | | | | LRRFIP1; | NM_001137551; |
| | | | | | LRRFIP1; | NM_004735; |
| | | | | | LRRFIP1 | NM_001137553 |
| cg14220544 | 16 | 89627035 | + | Island | RPL13; | NM_033251; |
| | | | | | RPL13; | NM_000977; |
| | | | | | SNORD68 | NR_002450 |
| cg14343652 | 7 | 130789013 | − | N_Shore | FLJ43663; | NR_015431; |
| | | | | | FLJ43663 | NR_024153 |
| cg14487595 | 6 | 43597054 | + | Island | MAD2L1BP; | NM_001003690; |
| | | | | | GTPBP2 | NM_019096 |
| cg14577211 | 12 | 48744457 | − | Island | ZNF641 | NM_152320 |
| cg14777056 | 1 | 109647163 | − | S_Shelf | | |
| cg14913969 | 18 | 19749138 | − | Island | GATA6 | NM_005257 |
| cg14966562 | 5 | 32444709 | + | Island | ZFR | NM_016107 |
| cg15052246 | 19 | 10225060 | − | Island | PPAN-P2RY11; | NM_001040664; |
| | | | | | P2RY11 | NM_002566 |
| cg15054873 | 6 | 152464791 | − | OpenSea | SYNE1; | NM_182961; |
| | | | | | SYNE1; | NM_133650; |
| | | | | | SYNE1; | NM_015293; |
| | | | | | SYNE1 | NM_033071 |
| cg15216078 | 10 | 134567188 | + | S_Shore | INPP5A | NM_005539 |
| cg15407819 | 19 | 41062024 | − | S_Shore | SPTBN4; | NM_025213; |
| | | | | | SPTBN4 | NM_020971 |
| cg15438794 | 11 | 1012101 | − | S_Shore | AP2A2 | NM_012305 |
| cg15481583 | 1 | 64620825 | − | OpenSea | ROR1 | NM_005012 |
| cg15524381 | 1 | 161860677 | − | OpenSea | ATF6 | NM_007348 |
| cg15580309 | 1 | 46814106 | − | OpenSea | NSUN4 | NM_199044 |
| cg15616511 | 11 | 45887415 | + | OpenSea | CRY2; | NM_001127457; |
| | | | | | CRY2 | NM_021117 |
| cg15685943 | 1 | 115632272 | − | Island | TSPAN2 | NM_005725 |
| cg15696376 | 19 | 1605678 | − | Island | UQCR | NM_006830 |
| cg15726426 | 1 | 111216839 | + | Island | KCNA3 | NM_002232 |
| cg15784784 | 11 | 66628295 | − | S_Shore | PC; | NM_022172; |
| | | | | | PC; | NM_000920; |
| | | | | | PC | NM_001040716 |
| cg15785704 | 17 | 30669010 | + | Island | C17orf75 | NM_022344 |
| cg15812222 | 8 | 57184678 | − | OpenSea | | |
| cg15822656 | 7 | 30321030 | − | N_Shelf | | |
| cg15850851 | 2 | 43446987 | + | OpenSea | | |
| cg16103681 | 8 | 42752335 | − | Island | HOOK3; | NM_032410; |
| | | | | | RNF170; | NR_027669; |
| | | | | | RNF170; | NM_001160224; |
| | | | | | RNF170; | NM_001160223; |
| | | | | | RNF170; | NM_030954; |
| | | | | | RNF170; | NR_027668; |
| | | | | | RNF170 | NM_001160225 |
| cg16174234 | 19 | 33463305 | + | Island | C19orf40; | NM_152266; |
| | | | | | CCDC123 | NM_032816 |
| cg16352283 | 1 | 27338978 | + | Island | FAM46B | NM_052943 |
| cg16422137 | 16 | 31191328 | − | Island | FUS; | NM_001170937; |
| | | | | | FUS; | NR_028388; |
| | | | | | FUS; | NM_004960; |
| | | | | | FUS | NM_001170634 |
| cg16451027 | 5 | 112042859 | − | N_Shore | APC | NM_001127511 |
| cg16588163 | 3 | 190251464 | − | OpenSea | IL1RAP; | NM_001167929; |
| | | | | | IL1RAP; | NM_001167931; |
| | | | | | IL1RAP; | NM_001167930; |
| | | | | | IL1RAP; | NM_001167928; |
| | | | | | IL1RAP; | NM_134470; |
| | | | | | IL1RAP | NM_002182 |
| cg16626420 | 5 | 140737111 | + | S_Shore | PCDHGA2; | NM_018915; |
| | | | | | PCDHGA4; | NM_018917; |
| | | | | | PCDHGA1; | NM_018912; |
| | | | | | PCDHGB1; | NM_018922; |
| | | | | | PCDHGA3; | NM_018916; |
| | | | | | PCDHGA4 | NM_032053 |
| cg16673904 | 17 | 7521996 | − | S_Shelf | SHBG | NR_027463 |
| cg16769376 | 14 | 24685222 | + | S_Shelf | MDP1; | NM_138476; |
| | | | | | MDP1 | NM_138476 |
| cg16782174 | 2 | 128410803 | − | Island | LIMS2; | NM_001136037; |
| | | | | | LIMS2; | NM_017980; |
| | | | | | LIMS2; | NM_001161403; |
| | | | | | LIMS2 | NM_001161404 |
| cg16838749 | 2 | 241263067 | + | S_Shore | | |
| cg16880597 | 12 | 12420514 | + | Island | LRP6 | NM_002336 |
| cg16911220 | 12 | 52695728 | − | Island | KRT86 | NM_002284 |
| cg16924102 | 4 | 20044588 | − | OpenSea | | |
| cg16980637 | 19 | 51815359 | − | Island | IGLON5 | NM_001101372 |
| cg17232883 | 11 | 59318136 | − | OpenSea | | |
| cg17315639 | 8 | 80877113 | − | OpenSea | MRPS28 | NM_014018 |
| cg17354052 | 16 | 23568699 | + | Island | UBFD1; | NM_019116; |
| | | | | | EARS2; | NM_001083614; |
| | | | | | EARS2 | NR_003501 |
| cg17364250 | 6 | 31138925 | + | OpenSea | POU5F1 | NM_002701 |
| cg17413194 | 17 | 63053996 | + | S_Shore | GNA13 | NM_006572 |
| cg17453603 | 11 | 93904908 | − | OpenSea | PANX1 | NM_015368 |
| cg17515155 | 1 | 11714790 | − | Island | FBXO2; | NM_012168; |
| | | | | | FBXO44; | NM_183413; |
| | | | | | FBXO44; | NM_001014765; |
| | | | | | FBXO44; | NM_033182; |
| | | | | | FBXO44 | NM_183412 |
| cg17515966 | 1 | 6265894 | + | Island | RNF207 | NM_207396 |
| cg17552093 | 17 | 79875985 | + | Island | SIRT7 | NM_016538 |
| cg17710536 | 7 | 43918101 | − | OpenSea | URGCP; | NM_001077664; |
| | | | | | URGCP; | NM_001077663; |
| | | | | | URGCP | NM_017920 |
| cg17757376 | 7 | 91570087 | − | Island | AKAP9; | NM_147185; |
| | | | | | AKAP9 | NM_005751 |
| cg17794169 | 10 | 123358013 | + | Island | FGFR2; | NM_000141; |
| | | | | | FGFR2; | NM_001144917; |
| | | | | | FGFR2; | NM_001144919; |
| | | | | | FGFR2; | NM_022970; |
| | | | | | FGFR2 | NM_001144918 |
| cg17797815 | 10 | 119806972 | + | S_Shore | CASC2; | NR_026940; |
| | | | | | RAB11FIP2; | NM_014904; |
| | | | | | CASC2; | NR_026939; |
| | | | | | CASC2 | NR_026941 |
| cg17802216 | 6 | 169026352 | − | OpenSea | SMOC2; | NM_022138; |
| | | | | | SMOC2 | NM_001166412 |
| cg17859882 | 6 | 58287694 | − | Island | GUSBL2 | NR_003660 |
| cg17891977 | 10 | 133449715 | + | Island | | |
| cg17972631 | 1 | 9970653 | − | Island | CTNNBIP1; | NM_001012329; |
| | | | | | CTNNBIP1 | NM_020248 |
| cg18231048 | 12 | 2113801 | + | Island | DCP1B | NM_152640 |
| cg18259003 | 17 | 72347925 | − | Island | KIF19 | NM_153209 |
| cg18320766 | 19 | 55598782 | − | Island | EPS8L1; | NM_017729; |
| | | | | | EPS8L1 | NM_133180 |
| cg18371471 | 14 | 101029794 | − | OpenSea | BEGAIN; | NM_001159531; |
| | | | | | BEGAIN | NM_020836 |
| cg18386828 | 11 | 1860235 | + | OpenSea | TNNI2; | NM_001145841; |
| | | | | | TNNI2; | NM_001145829; |
| | | | | | TNNI2; | NM_003282; |
| | | | | | TNNI2 | NM_003282 |
| cg18512446 | 18 | 77192641 | − | N_Shore | NFATC1; | NM_006162; |
| | | | | | NFATC1; | NM_172388; |
| | | | | | NFATC1; | NM_172389; |
| | | | | | NFATC1; | NM_172387; |
| | | | | | NFATC1 | NM_172390 |
| cg18710162 | 17 | 33390808 | − | OpenSea | RFFL; | NM_001017368; |
| | | | | | RFFL | NM_057178 |
| cg18716164 | 19 | 30019647 | + | Island | VSTM2B | NM_001146339 |
| cg18789261 | 12 | 82752393 | + | Island | C12orf26; | NM_032230; |
| | | | | | CCDC59; | NR_033192; |
| | | | | | CCDC59 | NM_014167 |
| cg18862171 | 11 | 66314367 | − | Island | ACTN3; | NM_001104; |
| | | | | | ZDHHC24 | NM_207340 |
| cg18961589 | 10 | 133598669 | + | OpenSea | | |
| cg18965213 | 15 | 62989995 | + | Island | TLN2 | NM_015059 |
| cg19244855 | 19 | 3029152 | − | Island | TLE2; | NM_003260; |
| | | | | | TLE2; | NM_003260; |
| | | | | | TLE2; | NM_001144761; |
| | | | | | TLE2 | NM_001144762 |
| cg19312085 | 1 | 247553836 | − | OpenSea | | |
| cg19324027 | 2 | 74699582 | − | Island | MRPL53 | NM_053050 |
| cg19454239 | 13 | 28430925 | + | OpenSea | | |
| cg19555331 | 4 | 147561900 | + | Island | POU4F2 | NM_004575 |
| cg19571004 | 10 | 135340850 | − | N_Shore | CYP2E1 | NM_000773 |
| cg19623237 | 17 | 77818582 | + | Island | | |
| cg19734801 | 5 | 131281112 | − | Island | | |
| cg19788371 | 8 | 143086298 | + | OpenSea | | |
| cg19789047 | 5 | 141659747 | + | OpenSea | | |
| cg19811425 | 6 | 31778701 | − | S_Shelf | HSPA1L | NM_005527 |
| cg19837824 | 16 | 2097351 | + | N_Shore | TSC2; | NM_001077183; |
| | | | | | NTHL1; | NM_002528; |
| | | | | | TSC2; | NM_000548; |
| | | | | | TSC2 | NM_001114382 |
| cg19864138 | 3 | 62351484 | − | N_Shelf | | |
| cg19926434 | 14 | 66975296 | − | Island | GPHN; | NM_020806; |
| | | | | | GPHN | NM_001024218 |
| cg20061722 | 1 | 233463492 | − | N_Shore | KIAA1804 | NM_032435 |
| cg20295949 | 8 | 101170029 | + | N_Shore | SPAG1; | NM_003114; |
| | | | | | SPAG1 | NM_172218 |
| cg20303995 | 10 | 104159500 | − | Island | NFKB2; | NM_001077494; |
| | | | | | NFKB2; | NM_001077493; |
| | | | | | NFKB2 | NM_002502 |
| cg20316284 | 22 | 38794946 | + | Island | LOC400927 | NR_002821 |
| cg20349305 | 11 | 75919698 | + | Island | | |
| cg20543544 | 10 | 81003657 | − | Island | ZMIZ1 | NM_020338 |
| cg20567895 | 1 | 95149580 | + | OpenSea | | |
| cg20645601 | 7 | 2742371 | + | Island | AMZ1 | NM_133463 |
| cg20789760 | 15 | 36163070 | − | OpenSea | | |
| cg20808462 | 4 | 2243252 | − | Island | HAUS3 | NM_024511 |
| cg20853569 | 17 | 79247697 | − | OpenSea | SLC38A10; | NM_001037984; |
| | | | | | SLC38A10 | NM_138570 |
| cg20867633 | 1 | 204183116 | + | OpenSea | GOLT1A; | NM_198447; |
| | | | | | GOLT1A | NM_198447 |
| cg20899379 | 15 | 50839463 | + | OpenSea | USP50 | NM_203494 |
| cg20933713 | 1 | 175713219 | + | OpenSea | TNR | NM_003285 |
| cg21164242 | 4 | 4250161 | − | Island | TMEM128 | NM_032927 |
| cg21173623 | 9 | 139685156 | − | N_Shore | TMEM141 | NM_032928 |
| cg21211413 | 15 | 89348748 | − | S_Shore | ACAN; | NM_013227; |
| | | | | | ACAN | NM_001135 |
| cg21249093 | 9 | 73737300 | + | OpenSea | TRPM3 | NM_001007471 |
| cg21253590 | 7 | 102073120 | − | N_Shore | ORAI2; | NM_001126340; |
| | | | | | ORAI2 | NM_032831 |
| cg21306321 | 19 | 869024 | − | N_Shore | MED16 | NM_005481 |
| cg21333964 | 11 | 118955461 | + | N_Shore | HMBS | NM_000190 |
| cg21355487 | 10 | 49688893 | − | S_Shore | ARHGAP22 | NM_021226 |
| cg21476940 | 1 | 160854954 | + | OpenSea | ITLN1; | NM_017625; |
| | | | | | ITLN1 | NM_017625 |
| cg21479731 | 4 | 2464579 | − | Island | | |
| cg21492308 | 2 | 3750128 | − | N_Shore | ALLC | NM_018436 |
| cg21492942 | 20 | 44718714 | − | Island | NCOA5 | NM_020967 |
| cg21679294 | 11 | 102217990 | − | Island | BIRC2; | NM_001166; |
| | | | | | BIRC2 | NM_001166 |
| cg21768956 | 17 | 927652 | − | S_Shore | ABR; | NM_021962; |
| | | | | | ABR; | NM_001092; |
| | | | | | ABR | NM_001159746 |
| cg21817187 | 17 | 26711117 | − | N_Shore | SARM1 | NM_015077 |
| cg22084428 | 3 | 98242050 | + | S_Shore | CLDND1; | NM_001040183; |
| | | | | | CLDND1; | NM_019895; |
| | | | | | CLDND1; | NM_001040182; |
| | | | | | CLDND1; | NM_001040181; |
| | | | | | CLDND1; | NM_001040199; |
| | | | | | CLDND1 | NM_001040200 |
| cg22226904 | 1 | 27819060 | − | S_Shelf | | |
| cg22406758 | 11 | 631684 | − | S_Shelf | | |
| cg22606129 | 7 | 33714254 | + | OpenSea | | |
| cg22681344 | 19 | 585501 | + | Island | | |
| cg22684151 | 12 | 130572394 | + | OpenSea | | |
| cg22694153 | 1 | 46768912 | + | Island | LRRC41; | NM_006369; |
| | | | | | UQCRH | NM_006004 |
| cg22715760 | 15 | 44069288 | + | Island | ELL3; | NM_025165; |
| | | | | | ELL3 | NM_025165 |
| cg22776392 | 1 | 180923569 | + | Island | | |
| cg22789605 | 12 | 51419164 | − | N_Shore | SLC11A2 | NM_000617 |
| cg22876894 | 20 | 1783624 | − | N_Shore | | |
| cg23115907 | 6 | 137817260 | − | N_Shore | | |
| cg23186422 | 6 | 139013826 | − | Island | | |
| cg23199335 | 3 | 177315882 | − | OpenSea | | |
| cg23334306 | 14 | 33945217 | − | OpenSea | NPAS3; | NM_001164749; |
| | | | | | NPAS3; | NM_173159; |
| | | | | | NPAS3; | NM_001165893; |
| | | | | | NPAS3 | NM_022123 |
| cg23435567 | 3 | 113346763 | + | OpenSea | SIDT1 | NM_017699 |
| cg23442198 | 4 | 187126114 | + | OpenSea | CYP4V2 | NM_207352 |
| cg23476065 | 7 | 151106043 | − | N_Shore | WDR86 | NM_198285 |
| cg23574061 | 2 | 66418555 | + | OpenSea | | |
| cg23657185 | 6 | 99272559 | + | Island | | |
| cg23670415 | 3 | 57583409 | − | Island | ARF4 | NM_001660 |
| cg23703963 | 14 | 105953300 | − | Island | CRIP1; | NM_001311; |
| | | | | | CRIP1 | NM_001311 |
| cg23768117 | 17 | 79134486 | + | S_Shore | AATK | NM_001080395 |
| cg23840481 | 16 | 4666699 | − | Island | | |
| cg23864993 | 4 | 8621266 | + | OpenSea | CPZ; | NM_001014447; |
| | | | | | CPZ; | NM_003652; |
| | | | | | CPZ | NM_001014448 |
| cg23891190 | 1 | 1822194 | − | Island | GNB1 | NM_002074 |
| cg23912217 | 10 | 81839159 | − | S_Shore | LOC219347; | NR_027430; |
| | | | | | LOC219347; | NR_027431; |
| | | | | | LOC219347; | NR_027428; |
| | | | | | C10orf57; | NM_025125; |
| | | | | | LOC219347; | NR_027432; |
| | | | | | LOC219347 | NR_027429 |
| cg23956119 | 1 | 228135123 | + | Island | WNT9A | NM_003395 |
| cg23993005 | 10 | 91091209 | + | OpenSea | IFIT3; | NM_001549; |
| | | | | | IFIT3 | NM_001031683 |
| cg23996302 | 1 | 201140803 | + | S_Shore | | |
| cg24435747 | 1 | 24514557 | − | S_Shore | IL28RA; | NM_173065; |
| | | | | | IL28RA; | NM_173064; |
| | | | | | IL28RA | NM_170743 |
| cg24530147 | 3 | 138763894 | − | Island | PRR23C | NM_001134657 |
| cg24629455 | 1 | 3163970 | − | Island | PRDM16; | NM_022114; |
| | | | | | PRDM16 | NM_199454 |
| cg24819967 | io | 135190860 | − | N_Shore | | |
| cg24844295 | 17 | 78821928 | + | S_Shelf | RPTOR; | NM_001163034; |
| | | | | | RPTOR | NM-020761 |
| cg24872425 | 3 | 138725335 | − | Island | PRR23A | NM_001134659 |
| cg24997589 | 7 | 148680113 | + | OpenSea | | |
| cg25233139 | 13 | 112187396 | + | OpenSea | | |
| cg25297849 | 2 | 219922998 | + | Island | IHH | NM_002181 |
| cg25322489 | 7 | 157412694 | − | N_Shore | PTPRN2; | NM_002847; |
| | | | | | PTPRN2; | NM_130842; |
| | | | | | PTPRN2 | NM_130843 |
| cg25351036 | 4 | 48018582 | + | Island | NIPAL1 | NM_207330 |
| cg25426716 | 5 | 125695699 | + | OpenSea | GRAMD3 | NM_001146319 |
| cg25500553 | 17 | 7366116 | − | Island | ZBTB4; | NM_001128833; |
| | | | | | ZBTB4 | NM_020899 |
| cg25563983 | 22 | 17517329 | − | Island | CECR7 | NR_015352 |
| cg25593948 | 6 | 50786670 | + | N_Shore | TFAP2B | NM_003221 |
| cg25652859 | 20 | 57427412 | − | N_Shore | GNAS; | NM_080425; |
| | | | | | GNAS; | NM_001077490; |
| | | | | | GNAS; | NM_016592; |
| | | | | | GNASAS | NR_002785 |
| cg25694156 | 15 | 69745050 | − | Island | RPLP1; | NM_213725; |
| | | | | | RPLP1 | NM_001003 |
| cg25821072 | 12 | 129252706 | − | OpenSea | | |
| cg25877009 | 8 | 142288517 | − | OpenSea | | |
| cg25906638 | 10 | 10914374 | + | OpenSea | | |
| cg26080305 | 17 | 26696372 | + | N_Shore | SEBOX; | NM_001083896; |
| | | | | | VTN | NM_000638 |
| cg26198430 | 6 | 166796458 | + | Island | BRP44L; | NM_016098; |
| | | | | | BRP44L | NM_016098 |
| cg26282792 | 19 | 58554479 | − | Island | ZSCAN1 | NM_182572 |
| cg26350143 | 11 | 2985721 | + | OpenSea | SNORA54; | NR_002982; |
| | | | | | NAP1L4 | NM_005969 |
| cg26373171 | 1 | 210001600 | − | S_Shore | C1orf107 | NM_014388 |
| cg26395211 | 5 | 140044315 | − | Island | WDR55 | NM_017706 |
| cg26428054 | 12 | 49484058 | − | Island | DHH | NM_021044 |
| cg26589069 | 16 | 4983989 | − | N_Shelf | PPL | NMJS02705 |
| cg26697583 | 15 | 89992598 | − | OpenSea | | |
| cg26758396 | 20 | 6104274 | + | S_Shore | FERMT1 | NM_017671 |
| cg26809210 | 2 | 74056290 | + | Island | STAMBP; | NM_006463; |
| | | | | | STAMBP; | NM_201647; |
| | | | | | STAMBP; | NM_213622; |
| | | | | | STAMBP | NM_213622 |
| cg26810230 | 10 | 134948300 | + | S_Shelf | | |
| cg26829101 | 12 | 119592094 | − | Island | SRRM4 | NM_194286 |
| cg26865201 | 12 | 32112162 | − | Island | C12orf35 | NM_018169 |
| cg26945050 | 2 | 27346434 | − | Island | ABHD1 | NM_032604 |
| cg26953232 | 6 | 32942494 | − | S_Shore | BRD2; | NM_001113182; |
| | | | | | BRD2 | NM_005104 |
| cg27158481 | 20 | 25128681 | − | N_Shore | LOC284798; | NR_027093; |
| | | | | | LOC284798; | NR_027092; |
| | | | | | LOC284798 | NR_027091 |
| cg27229613 | 1 | 229569046 | + | Island | ACTA1 | NM_001100 |
| cg27574066 | 17 | 26687382 | + | S_Shelf | TMEM199 | NM_152464 |
| cg27648567 | 13 | 37393923 | − | Island | RFXAP | NM_000538 |

| | | | Nearest | | |
|---|---|---|---|---|---|
| | UCSC Ref | | Gene | | |
| ID | Gene Group | distance | Symbol | kME | Module |
| cg00067600 | | 197941 | PDCD6IP | −0.15989 | grey |
| cg00157199 | | 60255 | FRG1BP | 0.007622 | grey |
| cg00181968 | TSS200 | 0 | FAM163A | 0.758502 | brown |
| cg00223950 | Body | 0 | UBR4 | 0.331118 | grey |
| cg00242358 | Body | 0 | CSPG5 | −0.082 | grey |
| cg00293191 | TSS200 | 126 | HCCAT5 | −0.04019 | grey |
| cg00367396 | | 755 | ATP6V0CP3 | 0.266755 | grey |
| cg00467202 | TSS200 | 45 | PHKG2 | 0.31042 | grey |
| cg00519002 | TSS1500 | 0 | MT1A | 0.311306 | grey |
| cg00632914 | Body | 0 | ZFP2 | −0.32571 | grey |
| cg00649515 | TSS1500 | 109 | E34GALT2 | 0.350119 | grey |
| cg00656860 | TSS1500 | 0 | SKOR1 | −0.09901 | grey |
| cg00660106 | | 0 | SHTN1 | 0.082529 | grey |
| cg00760321 | Body; | 0 | IQCC | 0.586655 | grey |
| | 1stExon | | | | |
| cg00807237 | Body | 0 | GET4 | −0.18469 | grey |
| cg00859655 | 5′UTR | 0 | CYB5R2 | −0.22918 | grey |
| cg01029238 | 1stExon; | 0 | UBE2Q2 | 0.240392 | grey |
| | TSS1500 | | | | |
| cg01048272 | 5′UTR | 0 | CCDC57 | 0.046286 | grey |
| cg01050736 | Body | 0 | HLA-DQA2 | −0.36288 | grey |
| cg01184449 | TSS1500; | 318 | EML4 | −0.08742 | grey |
| | TSS1500 | | | | |
| cg01225095 | TSS1500 | 435 | YARS2 | 0.22811 | grey |
| cg01395533 | TSS200; | 130 | MAGI1 | 0.273987 | grey |
| | TSS200; | | | | |
| | TSS200 | | | | |
| cg01399317 | Body | 0 | FAM83H | −0.02048 | grey |
| cg01405445 | 3'UTR | 0 | HSPB6 | −0.07442 | grey |
| cg01437917 | | 9 | VMO1 | 0.09954 | grey |
| cg01456989 | Body; | 0 | INCENP | −0.11112 | grey |
| | Body | | | | |
| cg01524860 | | 3617 | POU3F2 | 0.179917 | grey |
| cg01673669 | | 0 | FOXB1 | 0.030525 | grey |
| cg01678799 | Body; | 0 | CCNDBP1 | 0.717013 | green |
| | Body; | | | | |
| | Body; | | | | |
| | 5′UTR | | | | |
| cg01763636 | TSS1500 | 261 | EP400NL | −0.06891 | grey |
| cg01837362 | | 311701 | ALG10 | 0.011934 | grey |
| cg01915076 | TSS200 | 139 | DDN | 0.237859 | grey |
| cg01917669 | | 6983 | PCBP3 | −0.3323 | grey |
| cg01947695 | | 25731 | UBE2MP1 | 0.0219 | grey |
| cg02141498 | | 297306 | FOXL1 | 0.665487 | brown |
| cg02156314 | TSS200; | 0 | ZEB1-AS1 | 0.214239 | grey |
| | TSS1500; | | | | |
| | TSS200; | | | | |
| | Body; | | | | |
| | Body | | | | |
| cg02159111 | | 596 | ASB1 | −0.43724 | grey |
| cg02229605 | | 2966 | SIX1 | 0.690841 | brown |
| cg02247356 | TSS200 | 0 | ULBP3 | 0.017214 | grey |
| cg02253142 | 5′UTR; | 0 | TMOD2 | −0.09105 | grey |
| | 5′UTR | | | | |
| cg02259284 | TSS200 | 0 | ALCAM | 0.20826 | grey |
| cg02305687 | 1stExon | 0 | RXFP3 | −0.07313 | grey |
| cg02336461 | 1stExon; | 0 | CLTC | 0.189139 | grey |
| | 5′UTR | | | | |
| cg02386875 | Body | 0 | LRRC37BP1 | 0.093213 | grey |
| cg02451502 | | 34432 | LINC00424 | 0.662022 | blue |
| cg02601685 | TSS200 | 169 | SCPEP1 | 0.457568 | grey |
| cg02619599 | | 7271 | SLC6A3 | −0.21197 | grey |
| cg02627599 | | 7415 | SASH1 | −0.04747 | grey |
| cg02704931 | Body; | 0 | GCOM1 | −0.15765 | grey |
| | TSS1500; | | | | |
| | TSS1500; | | | | |
| | Body; | | | | |
| | TSS1500 | | | | |
| cg02792794 | Body | 0 | ACACB | 0.26923 | grey |
| cg02797036 | Body; | 0 | DBNL | 0.423987 | grey |
| | Body; | | | | |
| | Body | | | | |
| cg02847037 | TSS1500 | 220 | ZDHHC20 | 0.382361 | grey |
| cg02859655 | Body | 0 | PIP5K1C | −0.09834 | grey |
| cg02930078 | TSS200 | 0 | NUP153 | 0.374405 | grey |
| cg02953397 | Body | 0 | ACAP3 | −0.37953 | grey |
| cg03051116 | | 2245 | MAP3K8 | −0.10977 | grey |
| cg03163246 | Body | 0 | PPBPP2 | −0.13173 | grey |
| cg03296248 | Body | 0 | COL4A1 | −0.35193 | grey |
| cg03420242 | | 1291 | ZNF423 | 0.113665 | grey |
| cg03422070 | TSS1500; | 0 | KCNQ1 | −0.04027 | grey |
| | Body; | | | | |
| | Body | | | | |
| cg03440762 | | 292 | MSX2 | −0.04997 | grey |
| cg03521085 | Body; | 0 | CTBP1 | −0.34216 | grey |
| | Body | | | | |
| cg03565674 | Body | 0 | MOB2 | −0.07584 | grey |
| cg03585974 | | 2706 | TMEM239 | 0.790346 | green |
| cg03622371 | 5′UTR; | 0 | P2RY6 | 0.713764 | turquoise |
| | 5′UTR; | | | | |
| | 5′UTR; | | | | |
| | 5′UTR | | | | |
| cg03742273 | | 66433 | TTC34 | −0.0304 | grey |
| cg03830093 | Body | 0 | PIK3R6 | −0.36237 | grey |
| cg03909886 | | 220504 | LOC441666 | −0.25394 | grey |
| cg03933810 | TSS200 | 176 | MED11 | 0.282653 | grey |
| cg04001333 | 1stExon | 0 | FLVCR2 | 0.15896 | grey |
| cg04049508 | Body; | 0 | MMP2 | −0.41714 | grey |
| | Body | | | | |
| cg04080595 | TSS200; | 91 | PRDM16 | 0.743815 | brown |
| | TSS1500; | | | | |
| | TSS200; | | | | |
| | TSS1500 | | | | |
| cg04094193 | TSS1500; | 278 | YTHDC1 | −0.08451 | grey |
| | TSS1500 | | | | |
| cg04162647 | Body; | 0 | BCL11B | 0.187773 | grey |
| | Body | | | | |
| cg04177091 | | 0 | LMF1 | 0.053863 | grey |
| cg04267807 | TSS200 | 0 | SEMA3E | 0.192916 | grey |
| cg04335449 | TSS200 | 0 | RUVBL1 | 0 304579 | grey |
| cg04400533 | Body | 0 | ETV5 | 0.036132 | grey |
| cg04430996 | 5′UTR | 0 | CAPN15 | −0.16048 | grey |
| cg04433035 | 5′UTR; | 0 | HNF1B | −0.02847 | grey |
| | 5′UTR; | | | | |
| | 1stExon; | | | | |
| | 1stExon | | | | |
| cg04444415 | TSS200 | 172 | ATP13A4 | −0.07553 | grey |
| cg04893513 | | 897 | LMF1 | 0.681133 | yellow |
| cg05019671 | 5′UTR | 0 | LHFPL2 | 0.333471 | grey |
| cg05116255 | TSS1500 | 1001 | ANKRD30A | −0.04675 | grey |
| cg05158074 | TSS1500 | 224 | CCDC63 | 0.20905 | grey |
| cg052l3661 | Body | 0 | SLC6A18 | −0.0188 | grey |
| cg05232576 | Body | 0 | CPXM1 | 0.66083 | yellow |
| cg05241015 | TSS1500 | 584 | PXDN | 0.618787 | brown |
| cg05263760 | Body | 0 | PHACTR1 | −0.09652 | grey |
| cg05384646 | | 92155 | TJP1 | 0.110293 | grey |
| cg05431257 | Body | 0 | GDF7 | 0.116982 | grey |
| cg05498024 | | 5236 | WAC | 0.107115 | grey |
| cg05583103 | TSS200 | 106 | UPK1A | −0.31632 | grey |
| cg05592911 | | 14809 | IGHMBP2 | 0.17003 | grey |
| cg05627987 | TSS200 | 90 | SHANK1 | 0.039995 | grey |
| cg05872236 | | 10532 | CXXC1 | 0.020722 | grey |
| cg06018240 | Body | 0 | SLC4A2 | −0.24614 | grey |
| cg06046805 | Body; | 0 | PDE4D | −0.25777 | grey |
| | Body; | | | | |
| | Body | | | | |
| cg06138505 | TSS200 | 0 | MCM2 | 0.292737 | grey |
| cg06225979 | TSS200 | 35 | ZNF396 | 0.062358 | grey |
| cg06287140 | Body; | 0 | BCL7A | −0.21 | grey |
| | Body | | | | |
| cg06289444 | Body | 0 | THBS2 | −0.16453 | grey |
| cg06297356 | Body | 0 | LMF1 | 0.667852 | yellow |
| cg06395280 | TSS200 | 54 | FARP2 | 0.408794 | grey |
| cg06419850 | 3′UTR; | 0 | VMO1 | 0.142728 | grey |
| | 3′UTR; | | | | |
| | 3′UTR; | | | | |
| | Body | | | | |
| cg06466839 | TSS200 | 183 | ARHGAP32 | 0.071715 | grey |
| cg06514855 | | 1589 | MAP2K3 | 0.007744 | grey |
| cg06525670 | | 4082 | LINC00544 | 0.19715 | grey |
| cg06704589 | | 8890 | SPACA7 | 0.035056 | grey |
| cg06745507 | Body | 0 | ELLS | 0.238576 | grey |
| cg06765785 | TSS1500 | 1325 | H19 | −0.01025 | grey |
| cg06938878 | TSS1500 | 780 | CALCB | −0.03159 | grey |
| cg06940110 | | 26476 | DDX18 | 0.186286 | grey |
| cg06944693 | TSS1500 | 758 | ADAMTS8 | 0.020612 | grey |
| cg06980252 | Body | 0 | CREB3L2 | 0.69058 | green |
| cg07004386 | | 0 | CCDC177 | 0.037116 | grey |
| cg07327178 | | 51 | LOC729506 | −0.06472 | grey |
| cg07337446 | 3′UTR | 0 | CYP39A1 | 0.675203 | green |
| cg07464206 | | 27978 | SOX6 | −0.30335 | grey |
| cg07470512 | Body; | 0 | NECAB3 | −0.05151 | grey |
| | Body; | | | | |
| | 1stExon; | | | | |
| | 5′UTR | | | | |
| cg07592012 | Body | 0 | STRA13 | 0.653064 | blue |
| cg07632934 | TSS1500; | 884 | SNORD115-4 | 0.015242 | grey |
| | TSS1500; | | | | |
| | TSS1500; | | | | |
| | TSS1500 | | | | |
| cg07657332 | | 112412 | MBL2 | 0.056498 | grey |
| cg07664272 | | 173746 | LINC00616 | 0.645325 | yellow |
| cg07665718 | TSS1500; | 0 | RAB8A | −0.17203 | grey |
| | 3′UTR | | | | |
| cg07679834 | Body | 0 | ZNF14 | 0.678514 | green |
| cg07695826 | TSS1500 | 0 | C18orf54 | 0.151246 | grey |
| cg07802917 | TSS200; | 161 | MYLK | 0.285134 | grey |
| | TSS200; | | | | |
| | TSS200; | | | | |
| | TSS200 | | | | |
| cg08031958 | | 45400 | SLC19A1 | 0.045782 | grey |
| cg08134324 | Body; | 0 | LUC7L | −0.16455 | grey |
| | Body | | | | |
| cg08162465 | | 33784 | LINC01140 | 0.068915 | grey |
| cg08211853 | Body; | 0 | COLEC11 | −0.14006 | grey |
| | Body | | | | |
| cg08220120 | 1stExon; | 0 | PROB1 | 0.000985 | grey |
| | 3′UTR | | | | |
| cg08440920 | | 299 | CRNDE | 0.231724 | grey |
| cg08475853 | | 40517 | DEFB131 | −0.35877 | grey |
| cg08494157 | TSS200 | 0 | UNKL | 0.311165 | grey |
| cg08569350 | Body; | 0 | C7orf50 | −0.2968 | grey |
| | Body; | | | | |
| | Body | | | | |
| cg08627624 | | 11301 | PBLD | −0.21278 | grey |
| cg08686879 | 1stExon | 0 | CSF2 | 0.731829 | blue |
| cg08704196 | | 46551 | LOC100130700 | −0.08877 | grey |
| cg08791849 | 5′UTR | 0 | AKAP11 | 0.395276 | grey |
| cg08395260 | 5′UTR | 0 | RAB30 | 0.709922 | blue |
| cg08909157 | Body; | 0 | C9orf66 | 0.003085 | grey |
| | 1stExon; | | | | |
| | 5′UTR | | | | |
| cg08994789 | Body | 0 | LRRC37BP1 | −0.32069 | grey |
| cg09100014 | Body | 0 | IRF8 | 0.751946 | turquoise |
| cg09235723 | | 6907 | RNASEH2C | −0.21861 | grey |
| cg09266113 | TSS1500 | 857 | YIPF1 | −0.03482 | grey |
| cg09290175 | | 20620 | RPL3 | −0.4312 | grey |
| cg09298014 | | 25410 | PAPOLA | 0.287746 | grey |
| cg09330551 | Body; | 0 | KLK15 | 0.70852 | blue |
| | Body; | | | | |
| | Body | | | | |
| cg09379188 | 5′UTR | 0 | FAM110B | 0.67665 | green |
| cg09423312 | Body; | 0 | C7orf50 | −0.13053 | grey |
| | Body; | | | | |
| | Body | | | | |
| cg09440812 | TSS1500; | 0 | C11orf80 | 0.32835 | grey |
| | TSS1500; | | | | |
| | Body | | | | |
| cg09618694 | 1stExon; | 0 | CCDC66 | 0.424393 | grey |
| | 5′UTR; | | | | |
| | Body; | | | | |
| | 1stExon; | | | | |
| | 5′UTR | | | | |
| cg09622957 | TSS1500; | 706 | ADAMTSL4 | 0.237781 | grey |
| | TSS1500 | | | | |
| cg09894655 | | 49911 | C2orf27A | 0.124709 | grey |
| cg09928766 | Body | 0 | IGLON5 | −0.10724 | 9rey |
| cg09955084 | | 3569 | MRPL28 | −0.18761 | grey |
| cg09962377 | Body | 0 | RAB11FIP3 | 0.677227 | green |
| cg10052164 | Body | 0 | MOB2 | −0.1151 | 9rey |
| cg10081621 | | 0 | MCTP2 | −0.06433 | grey |
| cg10113910 | | 18783 | LINC01566 | −0.13948 | grey |
| cg10140536 | TSS1500 | 911 | USP29 | −0.13547 | grey |
| cg10194829 | TSS1500 | 249 | FGF17 | 0.073553 | grey |
| cg10212537 | TSS200; | 4 | UNC79 | 0.121085 | grey |
| | TSS200; | | | | |
| | TSS200 | | | | |
| cg10290200 | Body; | 0 | FLNC | −0.40989 | grey |
| | Body | | | | |
| cg10290276 | 1stExon; | 0 | ASCL2 | 0.693117 | brown |
| | 5′UTR | | | | |
| cg10318443 | 1stExon; | 0 | GLOD4 | 0.205555 | grey |
| | TSS200 | | | | |
| cg10334121 | TSS200; | 0 | CLDN18 | −0.1863 | grey |
| | Body | | | | |
| cg10436026 | Body; | 0 | SMAD9 | −0.07974 | grey |
| | Body | | | | |
| cg10460130 | Body; | 0 | DTYMK | 0.270457 | grey |
| | Body; | | | | |
| | Body | | | | |
| cg10536462 | Body | 0 | GAK | −0.34728 | grey |
| cg10682833 | | 2667 | SNRNP70 | −0.28695 | grey |
| cg10791023 | 3′UTR | 0 | CASR | −0.38262 | grey |
| cg10915739 | | 4229 | OLIG2 | −0.05976 | grey |
| cg10984252 | 5′UTR; | 0 | CASP7 | 0.304406 | grey |
| | TSS200; | | | | |
| | TSS200; | | | | |
| | TSS200 | | | | |
| cg10985158 | Body | 0 | NAV1 | 0.676825 | blue |
| cg11079278 | Body | 0 | SIAH3 | 0.24259 | grey |
| cg11165881 | TSS1500 | 229 | CHL1 | −0.01453 | grey |
| cg11461030 | | 20864 | FAAHP1 | 0.013685 | grey |
| cg11524248 | TSS1500 | 435 | CDX1 | −0.40466 | grey |
| cg11558474 | 5′UTR; | 0 | TMEM2 | 0.331122 | grey |
| | 5′UTR | | | | |
| cg11591485 | Body | 0 | SLC12A8 | −0.23497 | grey |
| cg11628781 | Body | 0 | SLC12A7 | −0.20447 | grey |
| cg11706080 | TSS200; | 26 | TRIB2 | 0.274887 | grey |
| | TSS200 | | | | |
| cg11710912 | Body | 0 | ARGLU1 | 0.082831 | grey |
| Cfl11835619 | Body; | 0 | ERBB3 | −0.27945 | grey |
| | Body | | | | |
| cg11902379 | Body; | 0 | CAMSAP3 | −0.43727 | grey |
| | Body | | | | |
| cg11915641 | 1stExon | 0 | SLC9A2 | 0.185727 | grey |
| cg12039242 | 5′UTR; | 0 | YWHAZ | 0.407015 | grey |
| | 5′UTR; | | | | |
| | 5′UTR; | | | | |
| | 5′UTR; | | | | |
| | 5′UTR; | | | | |
| | TSS200 | | | | |
| cg12039422 | | 128252 | LINC00670 | −0.12645 | grey |
| cg12118784 | | 3611 | GNG8 | −0.00426 | grey |
| cg12129897 | 3′UTR; | 0 | NXF1 | −0.07788 | grey |
| | 3′UTR; | | | | |
| | TSS1500 | | | | |
| cg12304381 | | 177519 | ZNF716 | −0.363 | grey |
| cg12346874 | | 0 | THSD7B | 0.422159 | grey |
| cg12373573 | 1stExon; | 0 | TSPAN19 | 0.190805 | grey |
| | 5′UTR; | | | | |
| | TSS200; | | | | |
| | TSS200 | | | | |
| cg12428906 | TSS200 | 119 | SERGEF | 0.676481 | grey |
| cg12451679 | TSS200 | 179 | ZC3H4 | 0.294494 | grey |
| cg12463883 | Body | 0 | FAM155A | 0.166212 | grey |
| cg12531953 | 5′UTR; | 0 | LYN | 0.619055 | brown |
| | 5′UTR | | | | |
| cg12594244 | TSS200 | 0 | LOXL1-AS1 | 0.752647 | turquoise |
| cg12712184 | | 185833 | SHH | −0.1189 | grey |
| cg12796916 | | 23012 | ALDH3B2 | −0.01827 | grey |
| cg12798700 | Body | 0 | HDDC2 | 0.152022 | grey |
| cg12851717 | Body | 0 | CLYBL | 0.677563 | green |
| cg12905865 | 5′UTR; | 0 | PLEKHG5 | 0.362716 | grey |
| | 5′UTR; | | | | |
| | Body; | | | | |
| | 5′UTR; | | | | |
| | Body | | | | |
| cg12922648 | Body | 0 | GTF2H4 | −0.2602 | grey |
| cg13092108 | Body | 0 | RPS6KA1 | 0.747812 | turquoise |
| cg13341153 | | 31377 | CASC2 | 0.061473 | grey |
| cg13357482 | TSS200 | 158 | POPDC3 | 0.601232 | grey |
| cg13372743 | Body | 0 | PXDNL | −0.25571 | grey |
| cg13627062 | | 4940 | NR2F2 | 0.335964 | grey |
| cg13629887 | | 1376 | CDKN1B | 0.382327 | grey |
| cg13702942 | Body; | 0 | ADAMTS2 | 0.417071 | grey |
| | Body | | | | |
| cg13707224 | | 6519 | DMRT2 | −0.04265 | grey |
| cg13782176 | Body; | 0 | PFKFB3 | −0.20054 | grey |
| | Body | | | | |
| cg13788515 | TSS200 | 76 | ZDBF2 | 0.624117 | brown |
| cg13900773 | 5′UTR | 0 | PIWIL1 | −0.01579 | grey |
| cg14016554 | Body | 0 | DDAH2 | −0.34443 | grey |
| cg14025831 | Body; | 0 | PANK2 | 0.754181 | blue |
| | 5′UTR; | | | | |
| | 5′UTR | | | | |
| cg14074117 | Body; | 0 | MEIOB | −0.04863 | grey |
| | Body | | | | |
| cg14076923 | Body; | 0 | GSN | −0.30181 | grey |
| | Body; | | | | |
| | Body; | | | | |
| | Body; | | | | |
| | Body; | | | | |
| | Body; | | | | |
| | Body; | | | | |
| | Body | | | | |
| cg14154547 | Body | 0 | RXRA | 0.724604 | blue |
| cg14162912 | Body; | 0 | LRRFIP1 | 0.125614 | grey |
| | TSS200; | | | | |
| | TSS200; | | | | |
| | TSS200; | | | | |
| | TSS200 | | | | |
| cg14220544 | TSS200; | 28 | RPL13 | 0.40832 | grey |
| | TSS200; | | | | |
| | TSS1500 | | | | |
| cg14343652 | Body; | 0 | LINC-PINT | 0.685645 | turquoise |
| | Body | | | | |
| cg14487595 | TSS1500; | 117 | GTPBP2 | −0.13234 | grey |
| | TSS200 | | | | |
| cg14577211 | TSS1500 | 0 | ZNF641 | 0.404971 | grey |
| cg14777056 | | 1408 | C1orf194 | 0.650539 | yellow |
| cg14913969 | TSS1500 | 208 | GATA6-AS1 | 0.33199 | grey |
| cg14966562 | Body | 0 | ZFR | 0.153665 | grey |
| cg15052246 | Body; | 0 | PPAN- | −0.08189 | grey |
| | Body | | P2RY11 | | |
| cg15054873 | Body; | 0 | SYNE1 | 0.691105 | green |
| | Body; | | | | |
| | Body; | | | | |
| | Body | | | | |
| cg15216078 | Body | 0 | INPP5A | −0.38313 | grey |
| cg15407819 | Body; | 0 | SPTBN4 | −0.2699 | grey |
| | Body | | | | |
| cg15438794 | 3′UTR | 0 | AP2A2 | −0.05583 | grey |
| cg15481583 | Body | 0 | ROR1 | −0.35497 | grey |
| cg15524381 | Body | 0 | ATF6 | 0.646396 | yellow |
| cg15580309 | Body | 0 | NSUN4 | −0.04283 | grey |
| cg15616511 | Body; | 0 | CRY2 | −0.2926 | grey |
| | Body | | | | |
| cg15685943 | TSS200 | 150 | TSPAN2 | 0.404002 | grey |
| cg15696376 | TSS200 | 194 | UQCR11 | 0.568993 | grey |
| cg15726426 | 1stExon | 0 | KCNA3 | 0.140052 | grey |
| cg15784784 | Body; | 0 | PC | −0.38847 | grey |
| | Body; | | | | |
| | Body | | | | |
| cg15785704 | Body | 0 | C17orf75 | 0.139747 | grey |
| cg15812222 | | 27890 | SDR16C5 | −0.22283 | grey |
| cg15822656 | | 2891 | ZNRF2 | −0.31092 | grey |
| cg15850851 | | 2552 | ZFP36L2 | 0.226771 | grey |
| cg16103681 | Body; | 0 | HOOK3 | 0.463274 | grey |
| | TSS1500; | | | | |
| | TSS1500; | | | | |
| | TSS1500; | | | | |
| | TSS1500; | | | | |
| | TSS1500; | | | | |
| | TSS1500 | | | | |
| cg16174234 | 5′UTR; | 0 | FAAP24 | 0.112812 | grey |
| | TSS1500 | | | | |
| cg16352283 | 1stExon | 0 | FAM46B | 0.711762 | brown |
| cg16422137 | TSS200; | 101 | FUS | 0.085395 | grey |
| | TSS200; | | | | |
| | TSS200; | | | | |
| | TSS200 | | | | |
| cg16451027 | TSS1500 | 341 | APC | −0.05918 | grey |
| cg16588163 | 5′UTR; | 0 | IL1RAP | 0.853804 | turquoise |
| | 5′UTR; | | | | |
| | 5′UTR; | | | | |
| | 5′UTR; | | | | |
| | 5′UTR; | | | | |
| | 5′UTR | | | | |
| cg16626420 | Body; | 0 | PCDHGA1 | −0.16172 | grey |
| | 1stExon; | | | | |
| | Body; | | | | |
| | Body; | | | | |
| | Body; | | | | |
| | 1stExon | | | | |
| cg16673904 | Body | 0 | SHBG | −0.23948 | grey |
| cg16769376 | 1stExon; | 0 | CHMP4A | 0.367554 | grey |
| | 5′UTR | | | | |
| cg16782174 | Body; | 0 | LIMS2 | −0.48313 | grey |
| | Body; | | | | |
| | Body; | | | | |
| | Body | | | | |
| cg16838749 | | 112046 | GPC1 | 0.048297 | grey |
| cg16880597 | TSS1500 | 702 | LRP6 | 0.197408 | grey |
| cg16911220 | 1stExon | 0 | KRT86 | −0.01308 | grey |
| cg16924102 | | 210645 | SLIT2 | −0.13498 | grey |
| cg16980637 | Body | 0 | IGLON5 | −0.08384 | grey |
| cg17232883 | | 23733 | OSBP | −0.08863 | grey |
| cg17315639 | Body | 0 | TPD52 | −0.36248 | grey |
| cg17354052 | TSS200; | 2 | EARS2 | 0.427973 | grey |
| | TSS200; | | | | |
| | TSS200 | | | | |
| cg17364250 | TSS1500 | 473 | POU5F1 | −0.41431 | grey |
| cg17413194 | TSS1500 | 1075 | GNA13 | −0.37741 | grey |
| cg17453603 | Body | 0 | PANX1 | −0.27115 | grey |
| cg17515155 | TSS200; | 0 | FBXO2 | 0.27334 | grey |
| | TSS200; | | | | |
| | TSS200; | | | | |
| | TSS200; | | | | |
| | 5′UTR | | | | |
| cg17515966 | TSS1500 | 293 | RNF207 | 0.140939 | grey |
| cg17552093 | 1stExon | 0 | SIRT7 | 0.417403 | grey |
| cg17710536 | Body; | 0 | URGCP- | −0.33144 | grey |
| | Body; | | MRPS24 | | |
| | Body | | | | |
| cg17757376 | TSS200; | 100 | AKAP9 | 0.232325 | grey |
| | TSS200 | | | | |
| cg17794169 | TSS200; | 40 | FGFR2 | 0.350889 | grey |
| | TSS200; | | | | |
| | TSS200; | | | | |
| | TSS200; | | | | |
| | TSS200 | | | | |
| cg17797815 | Body; | 0 | CASC2 | −0.16652 | grey |
| | TSS1500; | | | | |
| | Body; | | | | |
| | Body | | | | |
| cg17802216 | Body; | 0 | SMOC2 | −0.31136 | grey |
| | Body | | | | |
| cg17859882 | Body | 0 | GUSBP4 | 0.349064 | grey |
| cg17891977 | | 155017 | LINC01164 | −0.18073 | grey |
| cg17972631 | TSS1500; | 336 | CTNNBIP1 | 0.47424 | grey |
| | TSS1500 | | | | |
| cg18231048 | TSS200 | 123 | DCP1B | 0.238736 | grey |
| cg18259003 | Body | 0 | KIF19 | 0.072257 | grey |
| cg18320766 | Body; | 0 | EPS8L1 | 0.11965 | grey |
| | Body | | | | |
| cg18371471 | Body; | 0 | BEGAIN | 0.686457 | yellow |
| | Body | | | | |
| cg18386828 | TSS1500; | 0 | TNNI2 | −0.34194 | grey |
| | TSS1500; | | | | |
| | 1stExon; | | | | |
| | 5′UTR | | | | |
| cg18512446 | Body; | 0 | NFATC1 | −0.3585 | grey |
| | 5′UTR; | | | | |
| | Body; | | | | |
| | Body; | | | | |
| | Body | | | | |
| cg18710162 | TSS200; | 0 | RAD51L3- | 0.380455 | grey |
| | 5′UTR | | RFFL | | |
| cg18716164 | Body | 0 | VSTM2B | 0.321909 | grey |
| cg18789261 | 1stExon; | 0 | CCDC59 | 0.309958 | grey |
| | Body; | | | | |
| | TSS200 | | | | |
| cg18862171 | TSS200; | 0 | ACTN3 | 0.078951 | grey |
| | TSS1500 | | | | |
| cg18961589 | | 6063 | LINC01164 | −0.27649 | grey |
| cg18965213 | Body | 0 | TLN2 | −0.17852 | grey |
| cg19244855 | 5′UTR; | 0 | TLE2 | 0.407668 | grey |
| | 1stExon; | | | | |
| | Body; | | | | |
| | 5′UTR | | | | |
| cg19312085 | | 25620 | NLRP3 | 0.298264 | grey |
| cg19324027 | Body | 0 | CCDC142 | 0.395754 | grey |
| cg19454239 | | 62835 | GSX1 | −0.25975 | grey |
| cg19555331 | Body | 0 | POU4F2 | 0.762259 | brown |
| cg19571004 | TSS200 | 15 | CYP2E1 | −0.11383 | grey |
| cg19623237 | | 5368 | CBX4 | 0.223167 | grey |
| cg19734801 | | 0 | ACSL6 | −0.31221 | grey |
| cg19788371 | | 171400 | MIR4472-1 | −0.20214 | grey |
| cg19789047 | | 30243 | SPRY4 | −0.32964 | grey |
| cg19811425 | Body | 0 | HSPA1L | −0.46519 | grey |
| cg19837824 | TSS1500; | 0 | NTHL1 | 0.128871 | grey |
| | Body; | | | | |
| | TSS1500; | | | | |
| | TSS1500 | | | | |
| cg19864138 | | 3861 | FEZF2 | 0.142843 | grey |
| cg19926434 | 1stExon; | 0 | GPHN | 0.179774 | grey |
| | 1stExon | | | | |
| cg20061722 | TSS200 | 20 | KIAA1804 | 0.270252 | grey |
| cg20295949 | TSS1500; | 232 | SPAG1 | −0.06147 | grey |
| | TSS1500 | | | | |
| cg20303995 | Body; | 0 | NFKB2 | 0.577683 | grey |
| | Body; | | | | |
| | Body | | | | |
| cg20316284 | TSS200 | 14 | LOC400927 | −0.06215 | grey |
| cg20349305 | | 0 | WNT11 | 0.029457 | grey |
| cg20543544 | Body | 0 | ZMIZ1 | 0.177845 | grey |
| cg20567895 | | 136316 | SLC44A3 | −0.30851 | grey |
| cg20645601 | Body | 0 | AMZ1 | −0.02172 | grey |
| cg20789760 | | 11867 | DPH6-AS1 | −0.17112 | grey |
| cg20808462 | 5′UTR | 0 | POLN | 0.215513 | grey |
| cg20853569 | Body; | 0 | SLC38A10 | 0.783958 | yellow |
| | Body | | | | |
| cg20867633 | 5′UTR; | 0 | GOLT1A | 0.695124 | turquoise |
| | 1stExon | | | | |
| cg20899379 | TSS1500 | 560 | USP50 | 0.022974 | grey |
| cg20933713 | TSS1500 | 466 | TNR | −0.10151 | grey |
| cg21164242 | TSS1500 | 201 | TMEM128 | 0.158019 | grey |
| cg21173623 | TSS1500 | 619 | TMEM141 | 0.222464 | grey |
| cg21211413 | 5′UTR; | 0 | ACAN | −0.12591 | grey |
| | 5′UTR | | | | |
| cg21249093 | TSS1500 | 0 | TRPM3 | 0.093939 | grey |
| cg21253590 | TSS1500; | 855 | ORAI2 | 0.048433 | grey |
| | TSS1500 | | | | |
| cg21306321 | Body | 0 | MED16 | −0.41941 | grey |
| cg21333964 | TSS200 | 124 | HMBS | 0.251429 | grey |
| cg21355487 | Body | 0 | ARHGAP22 | 0.694593 | turquoise |
| cg21476940 | 5′UTR; | 0 | ITLN1 | 0.024766 | grey |
| | 1stExon | | | | |
| cg21479731 | | 0 | CFAP99 | 0.46904 | grey |
| cg21492308 | Body | 0 | ALLC | −0.33088 | grey |
| cg21492942 | TSS200 | 133 | NCOA5 | 0.217 | grey |
| cg21679294 | 1stExon; | 0 | BIRC2 | 0.15363 | grey |
| | 5′UTR | | | | |
| cg21768956 | Body; | 0 | ABR | 0.69098 | yellow |
| | Body; | | | | |
| | Body | | | | |
| cg21817187 | Body | 0 | SARM1 | 0.126213 | grey |
| cg22084428 | TSS200; | 139 | CLDND1 | 0.511436 | grey |
| | TSS200; | | | | |
| | TSS200; | | | | |
| | TSS200; | | | | |
| | TSS200; | | | | |
| | TSS200 | | | | |
| cg22226904 | | 2381 | WASF2 | 0.001307 | grey |
| cg22406758 | | 4510 | SCT | 0.726405 | yellow |
| cg22606129 | | 68573 | BBS9 | −0.24799 | grey |
| cg22681344 | | 2007 | BSG | −0.27538 | grey |
| cg22684151 | | 45506 | LOC100190940 | −0.10891 | grey |
| cg22694153 | 1stExon; | 0 | LRRC41 | 0.515427 | grey |
| | TSS1500 | | | | |
| cg22715760 | 5′UTR; | 0 | ELL3 | 0.294207 | grey |
| | 1stExon | | | | |
| cg22776392 | | 3646 | KIAA1614 | 0.003561 | grey |
| cg22789605 | 5′UTR | 0 | SLC11A2 | 0.10898 | grey |
| cg22876894 | | 23231 | LOC100289473 | 0.266582 | grey |
| cg23115907 | | 1728 | OLIG3 | 0.106244 | grey |
| cg23186422 | | 0 | FLJ46906 | 0.574472 | grey |
| cg23199335 | | 0 | LINC00578 | 0.322493 | grey |
| cg23334306 | Body; | 0 | NPAS3 | −0.30674 | grey |
| | Body; | | | | |
| | Body; | | | | |
| | Body | | | | |
| cg23435567 | 3′UTR | 0 | SIDT1 | −0.174 | grey |
| cg23442198 | Body | 0 | CYP4V2 | −0.32692 | grey |
| cg23476065 | Body | 0 | WDR86 | 0.041552 | grey |
| cg23574061 | | 166824 | MIR4778 | −0.26921 | grey |
| cg23657185 | | 10019 | POU3F2 | 0.130296 | grey |
| cg23670415 | TSS200 | 193 | ARF4 | 0.393583 | grey |
| cg23703963 | 1stExon; | 0 | CRIP1 | 0.023872 | grey |
| | 5′UTR | | | | |
| cg23768117 | Body | 0 | AATK | 0.667885 | turquoise |
| cg23840481 | | 1771 | UBALD1 | 0.150222 | grey |
| cg23864993 | Body; | 0 | CPZ | −0.05713 | grey |
| | Body; | | | | |
| | Body | | | | |
| cg23891190 | 5′UTR | 0 | GNB1 | 0.312402 | grey |
| cg23912217 | TSS1500; | 0 | TMEM254 | 0.200586 | grey |
| | TSS1500; | | | | |
| | TSS1500; | | | | |
| | Body; | | | | |
| | TSS1500; | | | | |
| | TSS1500 | | | | |
| cg23956119 | Body | 0 | WNT9A | 0.284182 | grey |
| cg23993005 | Body; | 0 | LIPA | −0.13713 | grey |
| | TSS1500 | | | | |
| cg23996302 | | 100 | TMEM9 | 0.081475 | grey |
| cg24435747 | TSS1500; | 791 | IFNLR1 | −0.15152 | grey |
| | TSS1500; | | | | |
| | TSS1500 | | | | |
| cg24530147 | TSS200 | 159 | PRR23C | −0.02357 | grey |
| cg24629455 | Body; | 0 | PRDM16 | −0.24504 | grey |
| | Body | | | | |
| cg24819967 | | 1879 | PAOX | −0.18941 | grey |
| cg24844295 | Body; | 0 | RPTOR | −0.25696 | grey |
| | Body | | | | |
| cg24872425 | TSS1500 | 224 | PRR23A | −0.01248 | grey |
| cg24997589 | | 20039 | PDIA4 | 0.111336 | grey |
| cg25233139 | | 190801 | TEX29 | 0.227067 | grey |
| cg25297849 | Body | 0 | IHH | 0.239644 | grey |
| cg25322489 | Body; | 0 | PTPRN2 | 0.671602 | blue |
| | Body; | | | | |
| | Body | | | | |
| cg25351036 | TSS1500 | 0 | CNGA1 | 0.328249 | grey |
| cg25426716 | TSS200 | 87 | GRAMD3 | −0.43536 | grey |
| cg25500553 | Body; | 0 | ZBTB4 | 0.107363 | grey |
| | Body | | | | |
| cg25563983 | TSS200 | 129 | CECR7 | 0.091715 | grey |
| cg25593948 | 1stExon | 0 | TFAP2B | −0.00057 | grey |
| cg25652859 | TSS1500; | 0 | GNAS | −0.03968 | grey |
| | TSS1500; | | | | |
| | 3′UTR; | | | | |
| | TSS1500 | | | | |
| cg25694156 | TSS200; | 107 | RPLP1 | 0.236111 | grey |
| | TSS200 | | | | |
| cg25821072 | | 25031 | SLC15A4 | 0.019278 | grey |
| cg25877009 | | 24291 | SLC45A4 | 0.783169 | turquoise |
| cg25906638 | | 62527 | LINC00710 | −0.14588 | grey |
| cg26080305 | TSS1500; | 0 | SARM1 | −0.31484 | grey |
| | Body | | | | |
| cg26198430 | 5′UTR; | 0 | MPC1 | 0.411989 | grey |
| | 1stExon | | | | |
| cg26282792 | Body | 0 | ZSCAN1 | 0.044336 | |
| cg26350143 | TSS1500; | 0 | NAP1L4 | 0.678076 | green |
| | Body | | | | |
| cg26373171 | Body | 0 | DIEXF | 0.002963 | grey |
| cg26395211 | TSS200 | 67 | WDR55 | 0.212155 | grey |
| cg26428054 | Body | 0 | DHH | −0.00658 | |
| cg26589069 | Body | 0 | PPL | 0.65839 | blue |
| cg26697583 | | 22038 | RHCG | −0.01733 | grey |
| cg26758396 | TSS200 | 82 | FERMT1 | −0.14253 | grey |
| cg26809210 | 5′UTR; | 0 | STAMBP | 0.318918 | grey |
| | 5′UTR; | | | | |
| | 5′UTR; | | | | |
| | 1stExon | | | | |
| cg26810230 | | 3120 | ADGRA1 | −0.30015 | grey |
| cg26829101 | Body | 0 | SRRM4 | 0.002873 | grey |
| cg26865201 | TSS200 | 189 | KIAA1551 | 0.492691 | grey |
| cg26945050 | TSS1500 | 221 | ABHD1 | 0.327905 | grey |
| cg26953232 | Body; | 0 | BRD2 | −0.0045 | grey |
| | Body | | | | |
| cg27158481 | TSS200; | 0 | LOC284798 | −0.10762 | grey |
| | Body; | | | | |
| | Body | | | | |
| cg27229613 | 5′UTR | 0 | ACTA1 | 0.125694 | grey |
| cg27574066 | Body | 0 | SARM1 | −0.27576 | grey |
| cg27648567 | 1stExon | 0 | RFXAP | 0.302349 | grey |

TABLE 17

| | | | | | UCSC | UCSC | UCSC | | Nearest |
|---|---|---|---|---|---|---|---|---|---|
| | | | | Relation | Ref Gene | Ref Gene | Ref Gene | | Gene |
| ID | chr | pos | strand | to Island | Name | Accession | Group | distance | Symbol |
| cg03380791 | 16 | 84163824 | − | OpenSea | HSDL1; HSDL1 | NM_031463; | Body; Body | 0 | HSDL1 |
| | | | | | | NM_001146051 | | | |
| cg27343836 | 16 | 86549678 | − | Island | | | | 1607 | FOXF1 |
| cg21904937 | 3 | 24563068 | − | Island | | | | 141 | MIR4792 |
| cg12260146 | 3 | 33840237 | + | Island | PDCD6IP; | NM_013374; | 1stExon; Body; | 0 | PDCD6IP |
| | | | | | PDCD6IP; | NM_027868; | 1stExon | | |
| | | | | | PDCD6IP | NM_001162429 | | | |
| cg05573133 | 3 | 46037470 | + | Island | FYCO1 | NM_024513 | TSS200 | 153 | FYCO1 |
| cg23247879 | 3 | 66306234 | + | OpenSea | SLC25A26; | NM_173471; | Body; Body; | 0 | SLC25A26 |
| | | | | | SLC25A26; | NM_028475; | Body | | |
| | | | | | SLC25A26 | NM_001164796 | | | |
| cg19862782 | 1 | 1153924 | − | Island | SDF4; SDF4 | NM_016176; | Body; Body | 0 | SDF4 |
| | | | | | | NM_016547 | | | |
| cg22012759 | 1 | 5816986 | − | OpenSea | | | | 105744 | MIR4689 |
| cg10555159 | 1 | 19971172 | + | Island | NBL1; NBL1 | NM_182744; | Body; 5′UTR | 0 | MINOS1-NBL1 |
| | | | | | | NM_005380 | | | |
| cg26220061 | 1 | 70819839 | − | Island | HHLA3; HHLA3; | NM_027404; | TSS1500; | 0 | ANKRD13C |
| | | | | | HHLA3; HHLA3; | NM_001036645; | TSS1500; | | |
| | | | | | ANKRD13C | NM_001031693; | TSS1500; | | |
| | | | | | | NM_001036646; | TSS1500; | | |
| | | | | | | NM_030816 | 1stExon | | |
| cg15422784 | 1 | 90023713 | + | OpenSea | LRRC8B; LRRC8B | NM_001134476; | 5′UTR; | 0 | LRRC8B |
| | | | | | | NM_015350 | TSS1500 | | |
| cg05053829 | 8 | 46856411 | + | OpenSea | | | | 896095 | LINC00293 |
| cg21196927 | 8 | 1.04E+08 | + | Island | FZD6; FZD6; | NM_003506; | 5′UTR; 1stExon; | 0 | FZD6 |
| | | | | | FZD6; FZD6; FZD6 | NM_001164616; | 1stExon; 5′UTR; | | |
| | | | | | | NM_003506; | 5′UTR | | |
| | | | | | | NM_001164615; | | | |
| | | | | | | NM_001164616 | | | |
| cg08515072 | 8 | 1.44E+08 | + | N_Shore | | | | 3560 | TOP1MT |
| cg07960693 | 14 | 89496795 | − | S_Shelf | | | | 125719 | FOXN3 |
| cg06948630 | 14 | 92588013 | + | Island | CPSF2; NDUFB1 | NM_017437; | TSS1500; | 0 | NDUFB1 |
| | | | | | | NM_004545 | 1stExon | | |
| cg24839718 | 19 | 37568953 | − | Island | ZNF420 | NM_144689 | TSS1500 | 427 | ZNF420 |
| cg07750118 | 19 | 41630815 | + | S_Shore | CYP2F1 | NM_000774 | Body | 0 | CYP2F1 |
| cg12026095 | 19 | 49468461 | − | Island | FTL | NM_000146 | TSS200 | 103 | FTL |
| cg04518808 | 19 | 54384822 | + | N_Shore | PRKCG | NM_002739 | TSS1500 | 643 | PRKCG |
| cg24851166 | 6 | 40400358 | + | OpenSea | LRFN2 | NM_020737 | Body | 0 | LRFN2 |
| cg13759676 | 6 | 1.29E+08 | − | OpenSea | PTPRK; PTPRK | NM_002844; | Body; Body | 0 | PTPRK |
| | | | | | | NM_001135648 | | | |
| cg17720267 | 12 | 6580010 | + | Island | VAMP1; VAMP1; | NM_016830; | TSS200; | 166 | VAMP1 |
| | | | | | VAMP1 | NM_199245; | TSS200; | | |
| | | | | | | NM_014231 | TSS200 | | |
| cg20731015 | 12 | 32112329 | − | Island | C12orf35 | NM_018169 | TSS200 | 22 | KIAA1551 |
| cg04558707 | 12 | 57578694 | + | N_Shore | LRP1 | NM_002332 | Body | 0 | LRP1 |
| cg26639561 | 12 | 58239492 | − | Island | CTDSP2 | NM_005730 | Body | 0 | CTDSP2 |
| cg13492159 | 12 | 1.03E+08 | + | S_Shore | BTBD11 | NM_001013072 | Body | 0 | BTBD11 |
| cg00434885 | 2 | 1.2E+08 | − | N_Shore | STEAP3; STEAP3; | NM_018234; | Body; Body; | 0 | STEAP3 |
| | | | | | STEAP3 | NM_182915; | Body | | |
| | | | | | | NM_001003410 | | | |
| cg09915950 | 2 | 2.02E+08 | − | OpenSea | AOX1 | NM_001159 | Body | 0 | AOX1 |
| cg03972606 | 2 | 2.35E+08 | + | OpenSea | HJURP | NM_018410 | Body | 0 | HJURP |
| cg00867510 | 2 | 2.4E+08 | + | N_Shore | | | | 39374 | HDAC4 |
| cg10677909 | 11 | 33890601 | + | Island | LMO2; LMO2; | NM_001142315; | Body; Body; | 0 | LMO2 |
| | | | | | LMO2 | NM_001142316; | Body | | |
| | | | | | | NM_005574 | | | |
| cg03054343 | 11 | 50238214 | + | Island | | | | 783 | LOC441601 |
| cg06129498 | 11 | 64058937 | + | Island | KCNK4; KCNK4 | NM_033310; | 5′UTR; 1stExon | 0 | KCNK4 |
| | | | | | | NM_033310 | | | |
| cg21033709 | 11 | 1.29E+08 | − | OpenSea | TP53AIP1; | NM_022112; | 1stExon; 5′UTR | 0 | TP53AIP1 |
| | | | | | TP53AIP1 | NM_022112 | | | |
| cg18687314 | 20 | 43733672 | − | S_Shelf | | | | 3918 | KCNS1 |
| cg06529134 | 20 | 57226134 | − | Island | STX16; STX16; | NM_001134772; | TSS200; | 173 | STX16 |
| | | | | | STX16; STX16 | NM_003763; | TSS200; | | |
| | | | | | | NM_001001433; | TSS200; | | |
| | | | | | | NM_001134773 | TSS200 | | |
| cg25626012 | 10 | 95721252 | − | OpenSea | PIPSL | NM_002319 | Body | 0 | PIPSL |
| cg21618418 | 18 | 77591684 | + | Island | | | | 31982 | KCNG2 |
| cg09436823 | 21 | 47575498 | + | OpenSea | FTCD; FTCD | NM_006657; | TSS200; | 16 | FTCD |
| | | | | | | NM_206965 | TSS200 | | |
| cg23162510 | 17 | 27054515 | − | N_Shore | TLCD1; TLCD1; | NM_001160407; | TSS1500; | 565 | TLCD1 |
| | | | | | NEKS | NM_138463; | TSS1500; | | |
| | | | | | | NM_178170 | TSS1500 | | |
| cg27316026 | 17 | 38859976 | − | OpenSea | KRT24; KRT24 | NM_019016; | 1stExon; 5′UTR | 0 | KRT24 |
| | | | | | | NM_019016 | | | |
| cg13278448 | 17 | 39538333 | + | OpenSea | KRT34 | NM_021013 | 1stExon | 0 | KRT34 |
| cg21637826 | 17 | 80845337 | − | OpenSea | TBCD | NM_005993 | Body | 0 | TBCD |
| cg16626480 | 22 | 25575426 | − | Island | KIAA1671 | NM_001145206 | Body | 0 | KIAA1671 |
| cg25330138 | 22 | 28031034 | + | N_Shelf | | | | 113229 | MN1 |
| cg07838388 | 13 | 45492839 | + | OpenSea | | | | 20543 | NUFIP1 |
| cg16325251 | 5 | 37883447 | + | OpenSea | | | | 7546 | GDNF-AS1 |
| cg06902071 | 5 | 1.35E+08 | + | N_Shore | | | | 7686 | NEUROG1 |
| cg08859406 | 5 | 1.48E+08 | − | OpenSea | | | | 4236 | SPINK7 |

TABLE 18

| | | | | | UCSC | UCSC | UCSC | | Nearest |
|---|---|---|---|---|---|---|---|---|---|
| | | | | Relation | Ref Gene | Ref Gene | Ref Gene | | Gene |
| ID | chr | pos | stran | to Island | Name | Accession | Group | distance | Symbol |
| cg00122851 | 16 | 1413159 | − | N_Shore | GNPTG | NM_032520 | 3′UTR | 0 | GNPTG |
| cg08098950 | 16 | 4033226 | + | S_Shelf | ADCY9 | NM_001116 | Body | 0 | ADCY9 |
| cg09477740 | 16 | 56677205 | − | Island | MT1DP; MT1DP | NM_003658; | TSS1500; | 392 | MT1DP |
| | | | | | | NM_027781 | TSS1500 | | |
| cg08066417 | 16 | 66413098 | + | OpenSea | CDH5 | NM_001795 | 5′UTR | 0 | CDH5 |
| cg01468907 | 16 | 84852577 | − | N_Shore | CRISPLD2 | NM_031476 | TSS1500 | 1008 | CRISPLD2 |
| cg07207789 | 16 | 84852909 | − | N_Shore | CRISPLD2 | NM_031476 | TSS1500 | 676 | CRISPLD2 |
| cg05493336 | 16 | 89469845 | − | OpenSea | ANKRD11 | NM_013275 | 5′UTR | 0 | ANKRD11 |
| cg07924503 | 16 | 89573631 | − | N_Shore | SPG7; SPG7 | NM_199367; | TSS1500; | 1172 | SPG7 |
| | | | | | | NM_003119 | TSS1500 | | |
| cg08822136 | 3 | 1.27E+08 | − | N_Shelf | | | | 2887 | PODXL2 |
| cg26867765 | 3 | 1.28E+08 | − | S_Shelf | RAB7A | NM_004637 | 5′UTR | 0 | RAB7A |
| cg24874638 | 3 | 1.56E+08 | + | S_Shore | C3orf33 | NM_173657 | TSS200 | 135 | C3orf33 |
| cg06066711 | 1 | 6662645 | − | Island | KLHL21 | NM_014851 | 1stExon | 0 | KLHL21 |
| cg18377941 | 1 | 94458645 | − | OpenSea | ABCA4 | NM_000350 | 3′UTR | 0 | ABCA4 |
| cg19383656 | 1 | 1.14E+08 | + | Island | RSBN1 | NM_018364 | TSS200 | 192 | RSBN1 |
| cg21813591 | 1 | 1.47E+08 | − | Island | BCL9 | NM_004326 | 5′UTR | 0 | BCL9 |
| cg06753662 | 1 | 2.07E+08 | + | Island | LGTN | NM_006893 | TSS200 | 95 | EIF2D |
| cg05941299 | 14 | 23095661 | − | OpenSea | | | | 14395 | ABHD4 |
| cg19578835 | 14 | 38067622 | − | Island | | | | 3296 | FOXA1 |
| cg08002381 | 9 | 86596124 | − | Island | HNRNPK; | NM_031262; | TSS1500; | 0 | RMI1 |
| | | | | | HNRNPK; | NM_002140; | TSS1500; | | |
| | | | | | HNRNPK; RMI1 | NM_031263; | TSS1500; | | |
| | | | | | | NM_024945 | 5′UTR | | |
| | 19 | 17407427 | − | S_Shelf | ABHD8 | NM_024527 | Body | 0 | ABHD8 |
| cg11199770 | 19 | 31841663 | − | Island | TSHZ3 | NM_020856 | TSS1500 | 1472 | TSHZ3 |
| cg26810281 | 6 | 31465714 | − | Island | MICE | NM_005931 | TSS200 | 0 | MICB |
| cg20042281 | 6 | 31515282 | − | OpenSea | NFKBIL1; | NM_001144961; | TSS200; 5′UTR; | 0 | NFKBIL1 |
| | | | | | NFKBIL1; | NM_001144963; | TSS1500; | | |
| | | | | | ATP6V1G2; | NM_130463; | TSS200; | | |
| | | | | | NFKBIL1; | NM_005007; | TSS1500; | | |
| | | | | | ATP6V1G2; | NM_138282; | 5′UTR | | |
| | | | | | NFKBIL1 | NM_001144962 | | | |
| cg27579984 | 12 | 49454806 | + | S_Shore | | | | 3660 | RHEBL1 |
| cg26214026 | 12 | 1.12E+08 | − | OpenSea | ATXN2 | NM_002973 | Body | 0 | ATXN2 |
| cg16460342 | 12 | 1.22E+08 | + | OpenSea | P2RX4 | NM_002560 | Body | 0 | P2RX4 |
| cg13772072 | 2 | 99070078 | − | OpenSea | INPP4A; INPP4A; | NM_001566; | 5′UTR; 5′UTR; | 0 | INPP4A |
| | | | | | INPP4A; INPP4A | NM_004027; | 5′UTR; 5′UTR | | |
| | | | | | | NM_001134224; | | | |
| | | | | | | NM_001134225 | | | |
| cg15357118 | 2 | 1.29E+08 | + | OpenSea | UGGT1; UGGT1 | NM_020120; | Body; Body | 0 | UGGT1 |
| | | | | | | NM_027671 | | | |
| cg19707243 | 2 | 1.52E+08 | + | OpenSea | | | | 62051 | RBM43 |
| cg00410370 | 2 | 1.75E+08 | − | Island | SP9 | NM_001145250 | Body | 0 | SP9 |
| cg21215272 | 2 | 1.78E+08 | + | Island | LOC100130691; | NM_026966; | TSS1500; | 0 | AGPS |
| | | | | | AGPS | NM_003659 | 1stExon | | |
| cg23719692 | 2 | 2.37E+08 | − | OpenSea | AGAP1; AGAP1 | NM_014914; | Body; Body | 0 | AGAP1 |
| | | | | | | NM_001037131 | | | |
| cg00050336 | 4 | 15377965 | + | OpenSea | C1QTNF7; | NM_001135170; | Body; 5′UTR | 0 | C1QTNF7 |
| | | | | | C1QTNF7 | NM_001135171 | | | |
| cg03203519 | 4 | 31292663 | + | OpenSea | | | | 144239 | PCDH7 |
| cg14909795 | 11 | 64851572 | + | Island | CDCA5; ZFPL1; | NM_080668; | 5′UTR; TSS200; | 0 | CDCA5 |
| | | | | | CDCA5 | NM_006782; | 1stExon | | |
| | | | | | | NM_080668 | | | |
| cg13234062 | 11 | 65668299 | + | S_Shore | FOSL1 | NM_005438 | TSS1500 | 301 | FOSL1 |
| cg22094205 | 11 | 74109076 | + | Island | PGM2L1 | NM_173582 | Body | 0 | PGM2L1 |
| cg04630321 | 11 | 74204613 | + | Island | LIPT2 | NM_001144869 | 1stExon | 0 | LIPT2 |
| cg15863552 | 11 | 1.3E+08 | − | Island | ST14 | NM_021978 | Body | 0 | ST14 |
| cg03415497 | 20 | 54933931 | − | N_Shore | C20orf108 | NM_080821 | TSS200 | 50 | FAM210B |
| cg03983808 | 10 | 43133352 | + | N_Shore | ZNF33B | NM_006955 | 5′UTR | 0 | ZNF33B |
| cg07875825 | 17 | 7210680 | − | Island | EIFSA; EIFSA; | NM_001143761; | TSS1500; | 0 | EIFSA |
| | | | | | EIFSA; EIFSA | NM_001143762; | TSS1500; Body; | | |
| | | | | | | NM_001143760; | TSS200 | | |
| | | | | | | NM_001970 | | | |
| cg05823643 | 17 | 56160706 | − | N_Shore | DYNLL2 | NM_080677 | TSS200 | 72 | DYNLL2 |
| cg01026192 | 7 | 2763915 | − | OpenSea | | | | 3824 | GNA12 |
| cg09378105 | 7 | 1.01E+08 | + | Island | | | | 0 | FIS1 |
| cg05102190 | 7 | 1.43E+08 | − | Island | ZYX; ZYX | NM_003461; | TSS200; | 0 | ZYX |
| | | | | | | NM_001010972 | TSS200 | | |
| cg24283289 | 22 | 19016892 | + | Island | | | | 0 | DGCR5 |
| cg00278131 | 13 | 51483857 | − | Island | RNASEH2B; | NM_024570; | TSS200; | 0 | RNASEH2B- |
| | | | | | RNASEH2B | NM_001142279 | TSS200 | | AS1 |
| cg16492226 | 5 | 83014308 | − | N_Shelf | HAPLN1 | NM_001884 | 5′UTR | 0 | HAPLN1 |
| cg22114568 | 5 | 1.77E+08 | + | OpenSea | | | | 366 | LOC728554 |

TABLE 19

| | | | | | UCSC | UCSC | UCSC | | Nearest |
|---|---|---|---|---|---|---|---|---|---|
| | | | | Relation | Ref Gene | Ref Gene | RefGene | | Gene |
| ID | chr | pos | strand | to Island | Name | Accession | Group | distance | Symbol |
| cg19067791 | 2 | 1.67E+08 | − | Island | TTC21B | NM_024753 | Body | 0 | TTC21B |
| cg26047920 | 5 | 1.41E+08 | + | Island | KIAA0141; | NM_014773; | TSS200; | 141 | KIAA0141 |
| | | | | | KIAA0141 | NM_001142603 | TSS200 | | |
| cg05090972 | 16 | 69788427 | − | N_Shore | NOB1 | NM_014062 | Body | 0 | NOB1 |
| cg04466253 | 10 | 27529581 | + | Island | ACBD5; | NM_145698; | Body; 5′UTR; | 0 | ACBD5 |
| | | | | | ACBD5; | NM_001042473; | Body | | |
| | | | | | ACBD5 | NM_024150 | | | |
| cg06829893 | 2 | 27346593 | + | Island | ABHD1 | NM_032604 | TSS200 | 62 | ABHD1 |
| cg06104667 | 7 | 72742068 | + | Island | TRIM50; | NM_178125; | 1stExon; | 0 | FKBP6 |
| | | | | | FKBP6; | NM_003602; | TSS1500; | | |
| | | | | | TRIM50; | NM_178125; | 5′UTR; | | |
| | | | | | FKBP6 | NM_001135211 | TSS200 | | |
| cg18776463 | 12 | 14924032 | + | S_Shore | HIST4H4; | NM_175054; | 1stExon; | 0 | HIST4H4 |
| | | | | | HIST4H4 | NM_175054 | 5′UTR | | |
| cg12741994 | 3 | 1.7E+08 | + | Island | CLDN11 | NM_005602 | Body | 0 | CLDN11 |
| cg13828227 | 9 | 1.31E+08 | + | Island | SH2D3C; | NM_005489; | Body; 5′UTR; | 0 | SH2D3C |
| | | | | | SH2D3C; | NM_001142531; | 5′UTR; Body; | | |
| | | | | | SH2D3C; | NM_001142532; | Body; Body | | |
| | | | | | SH2D3C; | NM_001142533; | | | |
| | | | | | SH2D3C; | NM_170600; | | | |
| | | | | | SH2D3C | NM_001142534 | | | |
| cg08905415 | 17 | 78831518 | − | Island | RPTOR; | NM_001163034; | Body; Body | 0 | RPTOR |
| | | | | | RPTOR | NM_020761 | | | |
| cg11371933 | 10 | 53459365 | − | Island | PRKG1; | NM_001098512; | Body; Body; | 0 | PRKG1 |
| | | | | | PRKG1; | NM_006258; | TSS200 | | |
| | | | | | CSTF2T | NM_015235 | | | |
| cg26246387 | 19 | 17791074 | + | Island | UNC13A | NM_001080421 | Body | 0 | UNC13A |
| cg24823993 | 22 | 42085003 | + | Island | NHP2L1 | NM_001003796 | TSS200 | 89 | SNU13 |
| cg03342084 | 11 | 43902343 | − | Island | ALKBH3 | NM_139178 | TSS200 | 12 | ALKBH3 |
| cg10697843 | 19 | 36236150 | + | Island | U2AF1L4; | NM_001040425; | Body; Body; | 0 | IGFLR1 |
| | | | | | U2AF1L4; | NM_144987; | TSS1500 | | |
| | | | | | PSENEN | NM_172341 | | | |
| cg00629117 | 16 | 1844932 | − | S_Shelf | IGFALS; | NM_027389; | TSS200; | 22 | IGFALS |
| | | | | | IGFALS; | NM_004970; | TSS1500; | | |
| | | | | | IGFALS | NM_001146006 | TSS1500 | | |
| cg13798290 | 1 | 1.14E+08 | − | Island | | | | 0 | LOC643441 |
| cg19254558 | 3 | 1.84E+08 | − | N_Shore | PARL; PARL | NM_001037639; | Body; Body | 0 | PARL |
| | | | | | | NM_018622 | | | |
| cg09183742 | 1 | 1.04E+08 | + | OpenSea | COL11A1; | NM_080629; | Body; Body; | 0 | COL11A1 |
| | | | | | COL11A1; | NM_080630; | Body; Body | | |
| | | | | | COL11A1; | NM_001168249; | | | |
| | | | | | COL11A1 | NM_001854 | | | |
| cg24528297 | 14 | 1.04E+08 | + | N_Shore | TRMT61A | NM_152307 | TSS200 | 146 | TRMT61A |
| cg05070571 | 13 | 1.14E+08 | − | N_Shelf | ATP11A; | NM_015205; | 3′UTR; Body | 0 | ATP11A |
| | | | | | ATP11A | NM_032189 | | | |
| cg02768197 | 17 | 80858983 | + | S_Shore | TBCD | NM_005993 | Body | 0 | TBCD |
| cg19769520 | 2 | 1659591 | − | OpenSea | PXDN | NM_012293 | Body | 0 | PXDN |
| cg26280685 | 11 | 62104020 | + | N_Shore | ASRGL1; | NM_001083926; | TSS1500; | 752 | ASRGL1 |
| | | | | | ASRGL1 | NM_025080 | TSS1500 | | |
| cg00424800 | 3 | 1.7E+08 | + | Island | GPR160 | NM_014373 | TSS200 | 131 | GPR160 |
| cg16314432 | 6 | 1.59E+08 | + | OpenSea | GTF2H5 | NM_207118 | Body | 0 | GTF2H5 |
| cg01000937 | 14 | 30730636 | − | OpenSea | | | | 297691 | G2E3 |
| cg27589796 | 5 | 1338514 | − | N_Shore | CLPTM1L | NM_030782 | Body | 0 | CLPTM1L |
| cg07242333 | 16 | 816874 | + | OpenSea | MSLN; MSLN | NM_005823; | Body; Body | 0 | MSLN |
| | | | | | | NM_013404 | | | |
| cg08127916 | 11 | 1.23E+08 | + | OpenSea | UBASH3B | NM_032873 | Body | 0 | UBASH3B |
| cg21010028 | 7 | 1969337 | − | N_Shore | MAD1L1; | NM_003550; | Body; Body; | 0 | MAD1L1 |
| | | | | | MAD1L1; | NM_001013837; | Body | | |
| | | | | | MAD1L1 | NM_001013836 | | | |
| cg15435932 | 2 | 46707626 | + | Island | LOC388946 | NM_001145051 | Body | 0 | TMEM247 |
| cg08282512 | 11 | 43918805 | − | OpenSea | LOC729799; | NM_026952; | TSS200; Body | 0 | ALKBH3 |
| | | | | | ALKBH3 | NM_139178 | | | |
| cg23889654 | 10 | 860862 | − | OpenSea | LARP4B | NM_015155 | Body | 0 | LARP4B |
| cg27126666 | 15 | 93353059 | − | Island | | | | 12720 | ASB9P1 |
| cg20633203 | 8 | 8239143 | + | OpenSea | PRAGMIN | NM_001080826 | 1stExon | 0 | SGK223 |
| cg05668205 | 19 | 16738998 | + | Island | MED26; | NM_004831; | 1stExon; | 0 | MED26 |
| | | | | | MED26 | NM_004831 | 5′UTR | | |
| cg20227766 | 1 | 27998703 | − | S_Shelf | IFI6; IFI6; IFI6; | NM_022873; | 1stExon; | 0 | IFI6 |
| | | | | | IFI6; IFI6; IFI6 | NM_022873; | 5′UTR; 5′UTR; | | |
| | | | | | | NM_002038; | 1stExon; | | |
| | | | | | | NM_002038; | 1stExon; | | |
| | | | | | | NM_022872; | 5′UTR | | |
| | | | | | | NM_022872 | | | |
| cg22798247 | 6 | 32807372 | + | S_Shore | TAP2; TAP2 | NM_018833; | TSS1500; | 824 | TAP2 |
| | | | | | | NM_000544 | TSS1500 | | |
| cg09714181 | 5 | 1.27E+08 | + | Island | PRRC1; | NM_130809; | 5′UTR; | 0 | PRRC1 |
| | | | | | PRRC1 | NM_130809 | 1stExon | | |
| cg18164942 | 14 | 75327747 | + | OpenSea | PROX2 | NM_001080408 | Body | 0 | PROX2 |
| cg12823408 | 11 | 1.2E+08 | + | OpenSea | ARHGEF12 | NM_015313 | 3′UTR | 0 | ARHGEF12 |
| cg02621694 | 4 | 81951472 | + | N_Shore | BMPS | NM_001201 | TSS1500 | 645 | BMP3 |
| cg21560413 | 2 | 27346765 | + | Island | ABHD1; | NM_032604; | 1stExon; | 0 | ABHD1 |
| | | | | | ABHD1 | NM_032604 | 5′UTR | | |
| cg04747517 | 3 | 1.7E+08 | + | Island | GPR160 | NM_014373 | TSS200 | 138 | GPR160 |
| cg26334518 | 17 | 19807272 | − | OpenSea | | | | 491 | AKAP10 |
| cg07011711 | 11 | 61467286 | − | OpenSea | DAGLA | NM_006133 | 5′UTR | 0 | DAGLA |
| cg24265719 | 8 | 1651101 | − | S_Shore | DLGAP2 | NM_004745 | 3′UTR | 0 | DLGAP2 |
| cg03242905 | 13 | 1.14E+08 | + | OpenSea | ADPRHL1; | NM_199162; | Body; Body | 0 | ADPRHL1 |
| | | | | | ADPRHL1 | NM_138430 | | | |
| cg21022792 | 6 | 29589798 | − | OpenSea | GABBR1; | NM_021904; | Body; Body; | 0 | GABBR1 |
| | | | | | GABBR1; | NM_021903; | Body | | |
| | | | | | GABBR1 | NM_001470 | | | |
| cg11845159 | 2 | 27346591 | + | Island | ABHD1 | NM_032604 | TSS200 | 64 | ABHD1 |
| cg03398910 | 10 | 35080979 | + | OpenSea | PARD3 | NM_019619 | Body | 0 | PARD3 |
| cg26825751 | 15 | 28983120 | − | Island | WHAMML2 | NM_026589 | Body | 0 | WHAMMP2 |
| cg06659629 | 4 | 660405 | − | S_Shelf | PDE6B; | NM_000283; | Body; Body; | 0 | PDE6B |
| | | | | | PDE6B; | NM_001145291; | Body | | |
| | | | | | PDE6B | NM_001145292 | | | |
| cg11519082 | 17 | 9559568 | + | OpenSea | USP43 | NM_153210 | Body | 0 | USP43 |
| cg26611508 | 13 | 88788987 | − | OpenSea | | | | 404095 | LINC00433 |
| cg14078687 | 9 | 1.36E+08 | + | N_Shelf | CEL | NM_001807 | Body | 0 | CEL |
| cg03172765 | 2 | 2.32E+08 | − | OpenSea | PSMD1 | NM_002807 | Body | 0 | PSMD1 |
| cg12046254 | 11 | 43902315 | − | Island | ALKBH3 | NM_139178 | TSS200 | 40 | ALKBH3 |
| cg17956609 | 11 | 43902458 | − | Island | ALKBH3; | NM_139178; | 1stExon; | 0 | ALKBH3 |
| | | | | | ALKBH3 | NM_139178 | 5′UTR | | |
| cg18690395 | 10 | 53459536 | − | Island | PRKG1; | NM_001098512; | Body; Body; | 0 | PRKG1 |
| | | | | | PRKG1; | NM_006258; | TSS200 | | |
| | | | | | CSTF2T | NM_015235 | | | |
| cg26970800 | 11 | 59614212 | − | OpenSea | GIF | NM_005142 | TSS1500 | 1237 | GIF |
| cg08874430 | 8 | 11659970 | + | Island | FDFT1 | NM_004462 | TSS1500 | 0 | FDFT1 |
| cg09854184 | 5 | 310135 | + | Island | AHRR; PDCD6 | NM_020731; | Body; Body | 0 | PDCD6 |
| | | | | | | NM_013232 | | | |
| cg08073652 | 6 | 43418057 | + | OpenSea | ABCC10 | NM_033450 | 3′UTR | 0 | ABCC10 |
| cg16400903 | 5 | 693638 | + | Island | TPPP | NM_007030 | TSS200 | 127 | TPPP |
| cg20119148 | 19 | 18344195 | − | Island | PDE4C | NM_000923 | 5′UTR | 0 | PDE4C |
| cg20009641 | 11 | 43902290 | − | Island | ALKBH3 | NM_139178 | TSS200 | 65 | ALKBH3 |
| cg14588422 | 10 | 35081084 | + | OpenSea | PARD3 | NM_019619 | Body | 0 | PARD3 |
| cg20326248 | 13 | 24217863 | + | OpenSea | TNFRSF19; | NM_018647; | Body; Body | 0 | TNFRSF19 |
| | | | | | TNFRSF19 | NM_148957 | | | |
| cg00522231 | 2 | 9549277 | − | OpenSea | ITGB1BP1; | NM_004763; | Body; Body | 0 | ITGB1BP1 |
| | | | | | ITGB1BP1 | NM_022334 | | | |
| cg16310003 | 12 | 1.22E+08 | + | OpenSea | HPD | NM_002150 | TSS1500 | 0 | HPD |
| cg14498674 | 1 | 41707653 | + | Island | SCMH1 | NM_012236 | 5′UTR | 0 | SCMH1 |
| cg18418335 | 19 | 2042088 | − | Island | MKNK2; | NM_199054; | Body; Body | 0 | MKNK2 |
| | | | | | MKNK2 | NM_017572 | | | |
| cg17747879 | 1 | 1.55E+08 | − | OpenSea | PBXIP1; | NM_020524; | TSS1500; | 0 | PYGO2 |
| | | | | | PYGO2 | NM_138300 | 3′UTR | | |
| cg06861562 | 5 | 60458483 | − | Island | C5orf43 | NM_001048249 | TSS200 | 180 | SMIM15 |
| cg08162465 | 1 | 87668671 | − | OpenSea | | | | 33784 | LINC01140 |
| cg16835097 | 17 | 73267459 | + | Island | MIF4GD | NM_020679 | TSS200 | 0 | LOC100287042 |
| cg27622722 | 8 | 67624757 | − | Island | SGK3 | NM_001033578 | TSS200 | 0 | C8orf44- |
| | | | | | | | | | SGK3 |
| cg16724148 | 1 | 1E+08 | + | OpenSea | AGL; AGL; | NM_000645; | TSS1500; | 0 | AGL |
| | | | | | AGL; AGL; | NM_000028; | Body; Body; | | |
| | | | | | AGL; AGL | NM_000642; | Body; Body; | | |
| | | | | | | NM_000646; | Body | | |
| | | | | | | NM_000644; | | | |
| | | | | | | NM_000643 | | | |
| cg18847089 | 7 | 676060 | + | Island | PRKAR1B; | NM_001164759; | Body; Body; | 0 | PRKAR1B |
| | | | | | PRKAR1B; | NM_001164762; | Body; Body; | | |
| | | | | | PRKAR1B; | NM_001164758; | Body; Body | | |
| | | | | | PRKAR1B; | NM_001164760; | | | |
| | | | | | PRKAR1B; | NM_001164761; | | | |
| | | | | | PRKAR1B | NM_002735 | | | |
| cg13600149 | 9 | 98079129 | + | N_Shore | FANCC | NM_000136 | 5′UTR | 0 | FANCC |
| cg27416898 | 8 | 33576374 | + | N_Shelf | | | | 118934 | DUSP26 |
| cg00669623 | 1 | 1655867 | + | Island | CDK11B; | NM_033487; | TSS200; | 0 | SLC35E2B |
| | | | | | CDK11B; | NM_033489; | TSS200; | | |
| | | | | | CDK11A; | NM_024011; | TSS200; | | |
| | | | | | CDK11B; | NM_033486; | TSS200; | | |
| | | | | | CDK11A; | NM_033529; | TSS200; | | |
| | | | | | CDK11B; | NM_033493; | TSS200; | | |
| | | | | | CDK11B; | NM_033488; | TSS200; | | |
| | | | | | CDK11B | NM_033492 | TSS200 | | |
| cg14793764 | 1 | 1110852 | + | S_Shore | TTLL10 | NM_001130045 | 5′UTR | 0 | TTLL10 |
| cg14498394 | 9 | 1.41E+08 | − | N_Shore | C9orf37; | NM_032937; | 5′UTR; | 0 | ARRDC1- |
| | | | | | EHMT1; | NM_024757; | TSS1500; | | AS1 |
| | | | | | EHMT1 | NM_001145527 | TSS1500 | | |
| cg02854695 | 19 | 50832032 | + | Island | KCNC3 | NM_004977 | 1stExon | 0 | KCNC3 |
| cg14474302 | 15 | 1.01E+08 | − | N_Shore | | | | 0 | PRKXP1 |
| cg03517918 | 1 | 19946398 | + | OpenSea | C1orf151 | NM_001032363 | Body | 0 | MINOS1- |
| | | | | | | | | | NBL1 |
| cg13354241 | 2 | 64447091 | − | OpenSea | | | | 14471 | UNC00309 |
| cg26458567 | 16 | 89557266 | − | Island | ANKRD11 | NM_013275 | TSS1500 | 296 | ANKRD11 |
| cg08385874 | 21 | 46352445 | − | Island | | | | 752 | LINC01547 |
| cg18700813 | 14 | 1.03E+08 | − | OpenSea | RAGE | NM_014226 | Body | 0 | MOK |
| cg11868634 | 7 | 1.56E+08 | − | OpenSea | | | | 192493 | SHH |
| cg08467261 | 16 | 53737935 | − | Island | RPGRIP1L; | NM_001127897; | TSS200; | 0 | FTO |
| | | | | | RPGRIP1L; | NM_015272; | TSS200; | | |
| | | | | | FTO; FTO | NM_001080432; | 1stExon; | | |
| | | | | | | NM_001080432 | 5′UTR | | |
| cg10312211 | 8 | 21867933 | + | Island | | | | 3836 | XPO7 |
| cg06642617 | 2 | 73461528 | − | Island | CCT7; CCT7; | NM_029402; | Body; Body; | 0 | CCT7 |
| | | | | | CCT7; CCT7; | NM_029403; | Body; Body; | | |
| | | | | | CCT7; C2orf7; | NM_001009570; | Body; | | |
| | | | | | CCT7 | NM_006429; | TSS1500; | | |
| | | | | | | NM_001166284; | 5′UTR | | |
| | | | | | | NM_032319; | | | |
| | | | | | | NM_001166285 | | | |
| cg25000382 | 16 | 2653501 | − | Island | LOC652276 | NM_015441 | Body | 0 | LOC652276 |
| cg26897297 | 13 | 49796434 | − | S_Shore | MLNR | NM_001507 | Body | 0 | MLNR |
| cg27136847 | 6 | 49705602 | − | OpenSea | CRISPS | NM_006061 | 5′UTR | 0 | CRISPS |

TABLE 20

| | | | | | UCSC | UCSC | UCSC | | Nearest |
|---|---|---|---|---|---|---|---|---|---|
| | | | | Relation | Ref Gene | Ref Gene | Ref Gene | | Gene |
| ID | chr | pos | strand | to Island | Name | Accession | Group | distance | Symbol |
| cg15407819 | 19 | 41062024 | − | S_Shore | SPTBN4; SPTBN4 | NM_025213; | Body; | 0 | SPTBN4 |
| | | | | | | NM_020971 | Body | | |
| cg10081621 | 15 | 94808566 | + | OpenSea | | | | 0 | MCTP2 |
| cg13702942 | 5 | 1.79E+08 | + | Island | ADAMTS2; | NM_021599; | Body; | 0 | ADAMTS2 |
| | | | | | ADAMTS2 | NM_014244 | Body | | |
| cg16174234 | 19 | 33463305 | + | Island | C19orf40; | NM_152266; | 5′UTR; | 0 | FAAP24 |
| | | | | | CCDC123 | NM_032816 | TSS1500 | | |
| cg10113910 | 16 | 34579002 | + | OpenSea | | | | 18783 | LINC01566 |
| cg22855405 | 1 | 32042037 | − | OpenSea | TINAGL1 | NM_022164 | TSS200 | 47 | TINAGL1 |
| cg27229613 | 1 | 2.3E+08 | + | Island | ACTA1 | NM_001100 | 5′UTR | 0 | ACTA1 |
| cg08540215 | 7 | 1.51E+08 | + | OpenSea | ABP1 | NM_001091 | TSS1500 | 0 | AOC1 |
| cg04009492 | 8 | 1.2E+08 | − | S_Shore | MAL2 | NM_052886 | Body | 0 | MAL2 |
| cg15481583 | 1 | 64620825 | − | OpenSea | ROR1 | NM_005012 | Body | 0 | ROR1 |
| cg00632914 | 5 | 1.78E+08 | + | OpenSea | ZFP2 | NM_030613 | Body | 0 | ZFP2 |
| cg12129897 | 11 | 62559856 | + | S_Shore | NXF1; NXF1; | NM_001081491; | 3′UTR; | 0 | NXF1 |
| | | | | | TMEM223 | NM_006362; | 3′UTR; | | |
| | | | | | | NM_001080501 | TSS1500 | | |
| cg10929921 | 8 | 1.04E+08 | + | S_Shore | FZD6; FZD6; FZD6 | NM_003506; | 5′UTR; | 0 | FZD6 |
| | | | | | | NM_001164615; | 5′UTR; | | |
| | | | | | | NM_001164616 | 5′UTR | | |
| cg03622371 | 11 | 73000436 | − | OpenSea | P2RY6; P2RY6; | NM_176797; | 5′UTR; | 0 | P2RY6 |
| | | | | | P2RY6; P2RY6 | NM_176796; | 5′UTR; | | |
| | | | | | | NM_004154; | 5′UTR; | | |
| | | | | | | NM_176798 | 5′UTR | | |
| cg01272202 | 5 | 1.13E+08 | + | OpenSea | MCC | NM_001085377 | Body | 0 | MCC |
| cg04001333 | 14 | 76045348 | + | S_Shore | FLVCR2 | NM_017791 | 1stExon | 0 | FLVCR2 |
| cg08162465 | 1 | 87668671 | − | OpenSea | | | | 33784 | LINC01140 |
| cg17430393 | 2 | 17722068 | + | S_Shore | VSNL1; VSNL1 | NM_003385; | 1stExon; | 0 | VSNL1 |
| | | | | | | NM_003385 | 5′UTR | | |
| cg18536042 | 19 | 40899779 | + | S_Shelf | PRX;PRX | NM_181882; | 3′UTR; | 0 | PRX |
| | | | | | | NM_020956 | 3′UTR | | |
| cg12451679 | 19 | 47617189 | − | S_Shore | ZC3H4 | NM_015168 | TSS200 | 179 | ZC3H4 |
| cg07597386 | 1 | 3028338 | − | Island | PRDM16; PRDM16 | NM_022114; | Body; | 0 | PRDM16 |
| | | | | | | NM_199454 | Body | | |
| cg09894655 | 2 | 1.32E+08 | − | N_Shore | | | | 49911 | C2orf27A |
| cg10915509 | 2 | 2.43E+08 | − | OpenSea | | | | 74893 | RTP5 |
| cg27266301 | 6 | 52931080 | − | S_Shore | FBXO9; FBXO9 | NM_033480; | Body; | 0 | FBXO9 |
| | | | | | | NM_033481 | 5′UTR | | |
| cg25351036 | 4 | 48018582 | + | Island | NIPAL1 | NM_207330 | TSS1500 | 0 | CNGA1 |
| cg04400533 | 3 | 1.86E+08 | + | OpenSea | ETV5 | NM_004454 | Body | 0 | ETV5 |
| cg00157199 | 20 | 29551622 | + | Island | | | | 60255 | FRG1BP |
| cg20295949 | 8 | 1.01E+08 | + | N_Shore | SPAG1; SPAG1 | NM_003114; | TSS1500; | 232 | SPAG1 |
| | | | | | | NM_172218 | TSS1500 | | |
| cg20480783 | 10 | 44186106 | − | Island | | | | 15958 | ZNF32-AS3 |
| cg26404511 | 1 | 24229575 | − | S_Shore | CNR2 | NM_001841 | 5′UTR | 0 | CNR2 |
| cg08134324 | 16 | 342012 | − | S_Shore | AXIN1; AXIN1 | NM_003502; | Body; | 0 | LUC7L |
| | | | | | | NM_181050 | Body | | |
| cg08895013 | 11 | 2468332 | − | S_Shore | KCNQ1 | NM_000218 | Body | 0 | KCNQ1 |
| cg15812222 | 8 | 57184678 | − | OpenSea | | | | 27890 | SDR16C5 |
| cg26809210 | 2 | 74056290 | + | Island | STAMBP; STAMBP; | NM_005463; | 5′UTR; | 0 | STAMBP |
| | | | | | STAMBP; STAMBP | NM_201647; | 5′UTR; | | |
| | | | | | | NM_213622; | 5′UTR; | | |
| | | | | | | NM_213622 | 1stExon | | |
| cg10212537 | 14 | 93799559 | − | Island | BTBD7; BTBD7; | NM_018167; | TSS200; | 4 | UNC79 |
| | | | | | KIAA1409 | NM_001002860; | TSS200; | | |
| | | | | | | NM_020818 | TSS200 | | |
| cg18320766 | 19 | 55598782 | − | Island | EPS8L1; EPS8L1 | NM_017729; | Body; | 0 | EPS8L1 |
| | | | | | | NM_133180 | Body | | |
| cg08494157 | 16 | 1429719 | + | Island | UNKL | NM_023076 | TSS200 | 0 | UNKL |
| cg06225979 | 18 | 32957337 | − | Island | ZNF396 | NM_145756 | TSS200 | 35 | ZNF396 |
| cg15580309 | 1 | 46814106 | − | OpenSea | NSUN4 | NM_199044 | Body | 0 | NSUN4 |
| cg08475853 | 4 | 9492758 | + | N_Shelf | | | | 40517 | DEFB131 |
| cg08031958 | 21 | 47009726 | − | N_Shore | | | | 45400 | SLC19A1 |
| cg06944693 | 11 | 1.3E+08 | + | S_Shore | ADAMTS8 | NM_007037 | TSS1500 | 758 | ADAMTS8 |
| cg01947695 | 16 | 34378069 | + | OpenSea | | | | 25731 | UBE2MP1 |
| cg03909886 | 10 | 42606808 | − | Island | | | | 220504 | LOC441666 |
| cg05527987 | 19 | 51220286 | + | Island | SHANK1 | NM_016148 | TSS200 | 90 | SHANK1 |
| cg17891977 | 10 | 1.33E+08 | + | Island | | | | 155017 | LINC01164 |
| cg21301440 | 17 | 74534550 | + | Island | CYGB | NM_134268 | TSS1500 | 0 | PRCD |
| cg08569350 | 7 | 1069812 | + | S_Shore | C7orf50; C7orf50; | NM_001134395; | Body; | 0 | C7orf50 |
| | | | | | C7orf50 | NM_032350; | Body; | | |
| | | | | | | NM_001134396 | Body | | |
| cg17797815 | 10 | 1.2E+08 | + | S_Shore | CASC2; | NR_026940; | Body; | 0 | CASC2 |
| | | | | | RAB11FIP2; | NM_014904; | TSS1500; | | |
| | | | | | CASC2; CASC2 | NR_026939; | Body; | | |
| | | | | | | NM_026941 | Body | | |
| cg21419585 | 1 | 1.18E+08 | + | OpenSea | FAM46C | NM_017709 | Body | 0 | FAM46C |
| cg09713850 | 6 | 32921791 | + | OpenSea | HLA-DMA | NM_006120 | TSS1500 | 891 | HLA-DMA |
| cg12161534 | 21 | 35743181 | + | OpenSea | KCNE2 | NM_172201 | 3′UTR | 0 | KCNE2 |
| cg02619599 | 5 | 1385632 | + | N_Shore | | | | 7271 | SLC6A3 |
| cg15052246 | 19 | 10225060 | − | Island | PPAN-P2RY11; | NM_001040664; | Body; | 0 | PPAN-P2RY11 |
| | | | | | P2RY11 | NM_002566 | Body | | |
| cg02386875 | 17 | 28927378 | + | Island | LRRC37B2 | NM_015341 | Body | 0 | LRRC37BP1 |
| cg23657185 | 6 | 99272559 | + | Island | | | | 10019 | POU3F2 |
| cg12121643 | 3 | 1.86E+08 | − | Island | DGKG; DGKG; | NM_001346; | Body; | 0 | DGKG |
| | | | | | DGKG | NM_001080744; | Body; | | |
| | | | | | | NM_001080745 | Body | | |
| cg07464206 | 11 | 15960015 | − | Island | | | | 27978 | SOX6 |
| cg02156314 | 10 | 31607685 | − | Island | ZEB1; ZEB1; ZEB1; | NR_024286; | TSS200; | 0 | ZEB1-AS1 |
| | | | | | ZEB1; LOC220930 | NM_030751; | TSS1500; | | |
| | | | | | | NR_024287; | TSS200; | | |
| | | | | | | NR_024285; | Body; | | |
| | | | | | | NM_024284 | Body | | |
| cg00807237 | 7 | 934156 | − | N_Shore | C7orf20 | NM_015949 | Body | 0 | GET4 |
| cg22789605 | 12 | 51419164 | − | N_Shore | SLC11A2 | NM_000617 | 5′UTR | 0 | SLC11A2 |
| cg25694156 | 15 | 69745050 | − | Island | RPLP1; RPLP1 | NM_213725; | TSS200; | 107 | RPLP1 |
| | | | | | | NM_001003 | TSS200 | | |
| cg02259284 | 3 | 1.05E+08 | + | N_Shore | ALCAM | NM_001627 | TSS200 | 0 | ALCAM |
| cg04049508 | 16 | 55535988 | − | OpenSea | MMP2; MMP2 | NM_001127891; | Body; | 0 | MMP2 |
| | | | | | | NM_004530 | Body | | |
| cg00660106 | 10 | 1.19E+08 | − | Island | | | | 0 | SHTN1 |
| cg04893513 | 16 | 902736 | − | N_Shore | | | | 897 | LMF1 |
| cg02820283 | 9 | 1.4E+08 | + | Island | ABCA2; ABCA2 | NM_001606; | Body; | 0 | ABCA2 |
| | | | | | | NM_212533 | Body | | |
| cg17762420 | 2 | 1.78E+08 | − | OpenSea | | | | 204805 | HNRNPA3 |
| cg24530147 | 3 | 1.39E+08 | − | Island | PRR23C | NM_001134657 | TSS200 | 159 | PRR23C |
| cg17515966 | 1 | 6265894 | + | Island | RNF207 | NM_207396 | TSS1500 | 293 | RNF207 |
| cg20543544 | 10 | 81003657 | − | Island | ZMIZ1 | NM_020338 | Body | 0 | ZMIZ1 |
| cg10334121 | 3 | 1.38E+08 | − | Island | CLDN13; CLDN18 | NM_016369; | TSS200; | 0 | CLDN18 |
| | | | | | | NM_001002026 | Body | | |
| cg11079278 | 13 | 46425210 | − | OpenSea | SIAH3 | NM_198849 | Body | 0 | SIAH3 |
| cg01524860 | 6 | 99290284 | + | Island | | | | 3617 | POU3F2 |
| cg04433035 | 17 | 36105064 | + | N_Shore | HNF1B; HNF1B; | NM_001165923; | 5′UTR; | 0 | HNF1B |
| | | | | | HNF1B; HNF1B | NM_000458; | 5′UTR; | | |
| | | | | | | NM_001165923; | 1stExon; | | |
| | | | | | | NM_000458 | 1stExon | | |
| cg18231048 | 12 | 2113801 | + | Island | DCP1B | NM_152640 | TSS200 | 123 | DCP1B |
| cg09100014 | 16 | 85937496 | + | OpenSea | IRF8 | NM_002163 | Body | 0 | IRF8 |
| cg06514855 | 17 | 21220141 | + | Island | | | | 1589 | MAP2K3 |
| cg13039585 | 5 | 1162588 | + | OpenSea | | | | 39120 | SLC6A19 |
| cg25781868 | 11 | 67289522 | + | S_Shore | CABP2; CABP2 | NM_016366; | Body; | 0 | CABP2 |
| | | | | | | NM_031204 | Body | | |
| cg09955084 | 16 | 413813 | + | S_Shelf | | | | 3569 | MRPL28 |
| cg16626420 | 5 | 1.41E+08 | + | S_Shore | PCDHGA2; | NM_018915; | Body; | 0 | PCDHGA1 |
| | | | | | PCDHGA4; | NM_018917; | 1stExon; | | |
| | | | | | PCDHGA1; | NM_018912; | Body; | | |
| | | | | | PCDHGB1; | NM_018922; | Body; | | |
| | | | | | PCDHGA3; | NM_018916; | Body; | | |
| | | | | | PCDHGA4 | NM_032053 | 1stExon | | |
| cg23574061 | 2 | 66418555 | + | OpenSea | | | | 166824 | MIR4778 |
| cg19254163 | 11 | 60623782 | − | S_Shelf | GPR44 | NM_004778 | TSS1500 | 337 | PTGDR2 |
| cg19837824 | 16 | 2097351 | + | N_Shore | TSC2; NTHL1; | NM_001077183; | TSS1500; | 0 | NTHL1 |
| | | | | | TSC2; TSC2 | NM_002528; | Body; | | |
| | | | | | | NM_000548; | TSS1500; | | |
| | | | | | | NM_001114382 | TSS1500 | | |
| cg21697944 | 19 | 49658797 | − | N_Shelf | HRC | NM_002152 | TSS200 | 115 | HRC |
| cg05431684 | 11 | 1.31E+08 | − | OpenSea | SNX19 | NM_014758 | Body | 0 | SNX19 |
| cg23115907 | 6 | 1.38E+08 | − | N_Shore | | | | 1728 | OLIG3 |
| cg06745507 | 15 | 44085196 | − | Island | SERF2 | NM_001018108 | Body | 0 | ELL3 |
| cg01029238 | 15 | 76136129 | − | Island | UBE2Q2; UBE2Q2 | NM_173469; | 1stExon; | 0 | UBE2Q2 |
| | | | | | | NM_001145335 | TSS1500 | | |
| cg04208928 | 1 | 2.26E+08 | + | S_Shore | LIN9 | NM_173083 | TSS1500 | 588 | LIN9 |
| cg11461030 | 1 | 46932239 | + | Island | | | | 20864 | FAAHP1 |
| cg15174823 | 6 | 33265886 | + | N_Shore | RGL2; RGL2 | NR_028387; | Body; | 0 | RGL2 |
| | | | | | | NM_004761 | Body | | |
| cg10255486 | 2 | 1.22E+08 | − | OpenSea | GLI2 | NM_005270 | Body | 0 | GLI2 |
| cg20789760 | 15 | 36163070 | − | OpenSea | | | | 11867 | DPH6-AS1 |
| cg04103645 | 13 | 51898948 | − | OpenSea | | | | 16218 | SERPINE3 |
| cg22257336 | 11 | 57191665 | − | N_Shelf | SLC43A3; | NM_014096; | Body; | 0 | SLC43A3 |
| | | | | | SLC43A3; SLC43A3 | NM_199329; | Body; | | |
| | | | | | | NM_017611 | Body | | |
| cg26829101 | 12 | 1.2E+08 | − | Island | SRRM4 | NM_194286 | Body | 0 | SRRM4 |
| cg17453603 | 11 | 93904908 | − | OpenSea | PANX1 | NM_015368 | Body | 0 | PANX1 |
| cg07004386 | 14 | 70040391 | − | N_Shore | | | | 0 | CCDC177 |
| cg05213661 | 5 | 1227968 | + | S_Shore | SLC6A18 | NM_182632 | Body | 0 | SLC6A18 |
| cg19864138 | 3 | 62351484 | − | N_Shelf | | | | 3861 | FEZF2 |
| cg17364250 | 6 | 31138925 | + | OpenSea | POU5F1 | NM_002701 | TSS1500 | 473 | POU5F1 |
| cg15283657 | 7 | 99066497 | − | N_Shelf | | | | 2672 | ATP5J2-PTCD1 |
| cg09719217 | 7 | 1.28E+08 | + | Island | PRRT4 | NM_001114726 | Body | 0 | PRRT4 |
| cg13762060 | 9 | 841559 | − | Island | DMRT1 | NM_021951 | TSS200 | 129 | DMRT1 |
| cg19811425 | 6 | 31778701 | − | S_Shelf | HSPA1L | NM_005527 | Body | 0 | HSPA1L |
| cg07291387 | 6 | 3445288 | − | OpenSea | SLC22A23; | NM_021945; | TSS200; | 0 | SLC22A23 |
| | | | | | SLC22A23 | NM_015482 | Body | | |
| cg25500553 | 17 | 7366116 | − | Island | ZBTB4; ZBTB4 | NM_001128833; | Body; | 0 | ZBTB4 |
| | | | | | | NM_020899 | Body | | |
| cg13900773 | 12 | 1.31E+08 | − | Island | PIWIL1 | NM_004764 | 5′UTR | 0 | PIWIL1 |
| cg16422137 | 16 | 31191328 | − | Island | FUS; FUS; FUS; | NM_001170937; | TSS200; | 101 | FUS |
| | | | | | FUS | NR_028388; | TSS200; | | |
| | | | | | | NM_004960; | TSS200; | | |
| | | | | | | NM_001170634 | TSS200 | | |
| cg20867633 | 1 | 2.04E+08 | + | OpenSea | GOLT1A; GOLT1A | NM_198447; | 5′UTR; | 0 | GOLT1A |
| | | | | | | NM_198447 | 1stExon | | |
| cg05116255 | 10 | 37413782 | − | N_shore | ANKRD30A | NM_052997 | TSS1500 | 1001 | ANKRD30A |
| cg08994789 | 17 | 28903642 | − | Island | LRRC37B2 | NR_015341 | Body | 0 | LRRC37BP1 |
| cg17861277 | 5 | 1.5E+08 | − | OpenSea | | | | 14338 | GPX3 |
| cg23435567 | 3 | 1.13E+08 | + | OpenSea | SIDT1 | NM_017699 | 3′UTR | 0 | SIDT1 |
| cg12304381 | 7 | 57710785 | + | OpenSea | | | | 177519 | ZNF716 |
| cg17170504 | 16 | 58283121 | + | N_Shore | CCDC113; | NM_014157; | TSS1500; | 717 | CCDC113 |
| | | | | | CCDC113 | NM_001142302 | TSS1500 | | |
| cg02579133 | 21 | 46031741 | + | OpenSea | KRTAP10-8; | NM_198695; | TSS1500; | 0 | TSPEAR |
| | | | | | C21orf29 | NM_144991 | Body | | |
| cg26810230 | 10 | 1.35E+08 | + | S_Shelf | | | | 3120 | ADGRA1 |
| cg13372743 | 8 | 52322134 | − | Island | PXDNL | NM_144651 | Body | 0 | PXDNL |
| cg17315639 | 8 | 80877113 | − | OpenSea | MRPS28 | NM_014018 | Body | 0 | TPD52 |
| cg24798914 | 2 | 1516240 | + | Island | TPO; TRO; TPO; | NM_175719; | Body; | 0 | TPO |
| | | | | | TPO | NM_000547; | Body; | | |
| | | | | | | NM_175721; | Body; | | |
| | | | | | | NM_175722 | Body | | |
| cg24629455 | 1 | 3163970 | − | Island | PRDM16; PRDM16 | NM_022114; | Body; | 0 | PRDM16 |
| | | | | | | NM_199454 | Body | | |
| cg23476065 | 7 | 1.51E+08 | − | N_Shore | WDR86 | NM_198285 | Body | 0 | WDR86 |
| cg15822858 | 6 | 1.68E+08 | + | OpenSea | | | | 42896 | UNC93A |
| cg08440920 | 16 | 54963401 | − | Island | | | | 299 | CRNDE |
| cg00649515 | 1 | 44456953 | − | N_Shore | CCDC24 | NM_152499 | TSS1500 | 109 | B4GALT2 |
| cg14817906 | 2 | 97466833 | − | OpenSea | CNNM4 | NM_020184 | Body | 0 | CNNM4 |
| cg01437917 | 17 | 4688569 | + | N_Shore | | | | 9 | VMO1 |
| cg00656860 | 15 | 68116607 | + | Island | LBXCOR1 | NM_001031807 | TSS1500 | 0 | SKOR1 |
| cg02859655 | 19 | 3648642 | − | Island | PIP5K1C | NM_012398 | Body | 0 | PIP5K1C |
| cg06704589 | 13 | 1.13E+08 | + | OpenSea | | | | 8890 | SPACA7 |
| cg20356510 | 17 | 76264518 | + | OpenSea | | | | 0 | LOC100996291 |
| cg26865201 | 12 | 32112162 | − | Island | C12orf35 | NM_018169 | TSS200 | 189 | KIAA1551 |
| cg03440762 | 5 | 1.74E+08 | + | N_Shore | | | | 292 | MSX2 |
| cg26198430 | 6 | 1.67E+08 | + | Island | BRP44L; BRP44L | NM_016098; | 5′UTR; | 0 | MPC1 |
| | | | | | | NM_016098 | 1stExon | | |
| cg14913969 | 18 | 19749138 | − | Island | GATA6 | NM_005257 | TSS1500 | 208 | GATA6-AS1 |
| cg24844295 | 17 | 78821928 | + | S_Shelf | RPTOR; RPTOR | NM_001163034; | Body; | 0 | RPTOR |
| | | | | | | NM_020761 | Body | | |
| cg05971894 | 12 | 54399254 | − | N_Shore | | | | 2133 | HOXC9 |
| cg25821072 | 12 | 1.29E+08 | − | OpenSea | | | | 25031 | SLC15A4 |
| cg06419850 | 17 | 4688683 | + | Island | VMO1; VMO1; | NM_001144941; | 3′UTR; | 0 | VMO1 |
| | | | | | VMO1; VMO1 | NM_001144940; | 3′UTR; | | |
| | | | | | | NM_001144939; | 3′UTR; | | |
| | | | | | | NM_182566 | Body | | |
| cg10460130 | 2 | 2.43E+08 | − | Island | DTYMK; DTYMK; | NM_001165031; | Body; | 0 | DTYMK |
| | | | | | DTYMK | NM_012145; | Body; | | |
| | | | | | | NM_033255 | Body | | |
| cg16580737 | 3 | 1.55E+08 | − | Island | MME; MME; | NM_007288; | TSS1500; | 0 | MME |
| | | | | | MME; MME | NM_007287; | TSS200; | | |
| | | | | | | NM_007289; | TSS1500; | | |
| | | | | | | NM_000902 | 5′UTR | | |
| cg03830093 | 17 | 8731697 | − | OpenSea | PIK3R6 | NM_001010855 | Body | 0 | PIK3R6 |
| cg12039422 | 17 | 12325031 | + | OpenSea | | | | 128252 | LINC00670 |
| cg09423312 | 7 | 1163549 | + | OpenSea | C7orf50; C7orf50; | NM_001134395; | Body; | 0 | C7orf50 |
| | | | | | C7orf50 | NM_032350; | Body; | | |
| | | | | | | NM_001134396 | Body | | |
| cg16769376 | 14 | 24685222 | + | S_Shelf | MDP1; MDP1 | NM_138476; | 1stExon; | 0 | CHMP4A |
| | | | | | | NM_138476 | 5′UTR | | |
| cg23199335 | 3 | 1.77E+08 | − | OpenSea | | | | 0 | LINC00578 |
| cg10791023 | 3 | 1.22E+08 | − | OpenSea | CASR | NM_000388 | 3′UTR | 0 | CASR |
| cg08220120 | 5 | 1.39E+08 | − | Island | LOC389333; | NM_001161546; | 1stExon; | 0 | PROB1 |
| | | | | | LOC389333 | NM_001161546 | 3′UTR | | |
| cg24872425 | 3 | 1.39E+08 | − | Island | PRR23A | NM_001134659 | TSS1500 | 224 | PRR23A |
| cg25563983 | 22 | 17517329 | − | Island | CECR7 | NM_015352 | TSS200 | 129 | CECR7 |
| cg14154547 | 9 | 1.37E+08 | − | OpenSea | RXRA | NM_002957 | Body | 0 | RXRA |
| cg24495350 | 15 | 72651198 | + | OpenSea | HEXA | NM_000520 | Body | 0 | CELF6 |
| cg04335449 | 3 | 1.28E+08 | − | Island | RUVBL1 | NM_003707 | TSS200 | 0 | RUVBL1 |
| cg21173623 | 9 | 1.4E+08 | − | N_Shore | TMEM141 | NM_032928 | TSS1500 | 619 | TMEM141 |
| cg09618694 | 3 | 56591190 | − | Island | CCDC66; CCDC66; | NM_001012506; | 1stExon; | 0 | CCDC66 |
| | | | | | CCDC66; CCDC66; | NM_001141947; | 5′UTR; | | |
| | | | | | CCDC66 | NM_024460; | Body; | | |
| | | | | | | NM_001141947; | 1stExon; | | |
| | | | | | | NM_001012506 | 5′UTR | | |
| cg14487595 | 6 | 43597054 | + | Island | MAD2L1BP; | NM_001003690; | TSS1500; | 117 | GTPBP2 |
| | | | | | GTPBP2 | NM_019096 | TSS200 | | |
| cg09298014 | 14 | 97058864 | + | N_Shore | | | | 25410 | PAPOLA |
| cg04162647 | 14 | 99734598 | − | N_Shore | BCL11B; BCL11B | NM_022898; | Body; | 0 | BCL11B |
| | | | | | | NM_138576 | Body | | |
| cg15665941 | 2 | 2.43E+08 | − | OpenSea | | | | 91721 | LOC728323 |
| cg26080305 | 17 | 26696372 | + | N_Shore | SEBOX; VTN | NM_001083896; | TSS1500; | 0 | SARM1 |
| | | | | | | NM_000638 | Body | | |
| cg21476940 | 1 | 1.61E+08 | + | OpenSea | ITLN1; ITLN1 | NM_017625; | 5′UTR; | 0 | ITLN1 |
| | | | | | | NM_017625 | 1stExon | | |
| cg00097357 | 12 | 33591336 | − | N_Shore | SYT10 | NM_198992 | Body | 0 | SYT10 |
| cg04835489 | 1 | 52624196 | + | OpenSea | ZFYVE9; ZFYVE9; | NM_007323; | 5′UTR; | 0 | ZFYVE9 |
| | | | | | ZFYVE9 | NM_004799; | 5′UTR; | | |
| | | | | | | NM_007324 | 5′UTR | | |
| cg10290276 | 11 | 2291754 | − | Island | ASCL2; ASCL2 | NM_005170; | 1stExon; | 0 | ASCL2 |
| | | | | | | NM_005170 | 5′UTR | | |
| cg07665718 | 19 | 16244068 | − | OpenSea | HSH2D; RAB8A | NM_032855; | TSS1500; | 0 | RAB8A |
| | | | | | | NM_005370 | 3′UTR | | |
| cg05262191 | 1 | 1714271 | − | S_Shore | | | | 2452 | GNB1 |
| cg17468855 | 1 | 2336721 | + | N_Shelf | PEX10; RER1; | NM_002617; | 3′UTR; | 0 | RER1 |
| | | | | | PEX10 | NM_007033; | 3′UTR; | | |
| | | | | | | NM_153818 | 3′UTR | | |
| cg03439958 | 2 | 1.02E+08 | − | OpenSea | NPAS2 | NM_002518 | 3′UTR | 0 | NPAS2 |
| cg03479278 | 7 | 1.07E+08 | + | N_Shore | BCAP29; BCAP29 | NM_018844; | TSS1500; | 209 | DUS4L |
| | | | | | | NR_027830 | TSS1500 | | |
| cg12905865 | 1 | 6545814 | + | S_Shore | PLEKHG5; | NM_020631; | 5′UTR; | 0 | PLEKHG5 |
| | | | | | PLEKHG5; | NM_001042665; | 5′UTR; | | |
| | | | | | PLEKHG5; | NM_198681; | Body; | | |
| | | | | | PLEKHG5; | NM_001042664; | 5′UTR; | | |
| | | | | | PLEKHG5 | NM_001042663 | Body | | |
| cg12163462 | 14 | 1.02E+08 | + | N_Shelf | | | | 12265 | DYNC1H1 |
| cg17710536 | 7 | 43918101 | − | OpenSea | URGCP; URGCP; | NM_001077664; | Body; | 0 | URGCP- |
| | | | | | URGCP | NM_001077663; | Body; | | MRPS24 |
| | | | | | | NM_017920 | Body | | |
| cg04094193 | 4 | 69216103 | − | S_Shore | YTHDC1; YTHDC1 | NM_133370; | TSS1500; | 278 | YTHDC1 |
| | | | | | | NM_001031732 | TSS1500 | | |
| cg21164242 | 4 | 4250161 | − | Island | TMEM128 | NM_032927 | TSS1500 | 201 | TMEM128 |
| cg09473282 | 6 | 35115955 | + | OpenSea | | | | 6767 | TCP11 |
| cg01399317 | 8 | 1.45E+08 | − | Island | FAM83H | NM_198488 | Body | 0 | FAM83H |
| cg02229605 | 14 | 61119122 | + | S_Shelf | | | | 2966 | SIX1 |
| cg13594630 | 12 | 32108895 | − | N_Shelf | | | | 3456 | KIAA1551 |
| cg19244855 | 19 | 3029152 | − | Island | TLE2; TLE2; TLE2; | NM_003260; | 5′UTR; | 0 | TLE2 |
| | | | | | TLE2 | NM_003260; | 1stExon; | | |
| | | | | | | NM_001144761; | Body; | | |
| | | | | | | NM_001144762 | 5′UTR | | |
| cg27158481 | 20 | 25128681 | − | N_Shore | LOC284798; | NR_027093; | TSS200; | 0 | LOC284798 |
| | | | | | LOC284798; | NR_027092; | Body; | | |
| | | | | | LOC284798 | NM_027091 | Body | | |
| cg15696376 | 19 | 1605678 | − | Island | UQCR | NM_006830 | TSS200 | 194 | UQCR11 |
| cg02247356 | 6 | 1.5E+08 | − | Island | ULBP3 | NM_024518 | TSS200 | 0 | ULBP3 |
| cg15054873 | 6 | 1.52E+08 | − | OpenSea | SYNE1; SYNE1; | NM_182961; | Body; | 0 | SYNE1 |
| | | | | | SYNE1; SYNE1 | NM_133650; | Body; | | |
| | | | | | | NM_015293; | Body; | | |
| | | | | | | NM_033071 | Body | | |
| cg21817187 | 17 | 26711117 | − | N_Shore | SARM1 | NM_015077 | Body | 0 | SARM1 |
| cg18716164 | 19 | 30019647 | + | Island | VSTM2B | NM_001146339 | Body | 0 | VSTM2B |
| cg07632934 | 15 | 25422945 | − | OpenSea | SNORD115-9; | NR_003301; | TSS1500; | 884 | SNORD115-4 |
| | | | | | SNORD115-5; | NR_003297; | TSS1500; | | |
| | | | | | SNORD115-12; | NR_003304; | TSS1500; | | |
| | | | | | SNORD115-10 | NM_003302 | TSS1500 | | |
| cg23891190 | 1 | 1822194 | − | Island | GNB1 | NM_002074 | 5′UTR | 0 | GNB1 |
| cg15850851 | 2 | 43446987 | + | OpenSea | | | | 2552 | ZFP36L2 |
| cg21333964 | 11 | 1.19E+08 | + | N_Shore | HMBS | NM_000190 | TSS200 | 124 | HMBS |
| cg15726426 | 1 | 1.11E+08 | + | Island | KCNA3 | NM_002232 | 1stExon | 0 | KCNA3 |
| cg25322489 | 7 | 1.57E+08 | − | N_Shore | PTPRN2; PTPRN2; | NM_002847; | Body; | 0 | PTPRN2 |
| | | | | | PTPRN2 | NM_130842; | Body; | | |
| | | | | | | NM_130843 | Body | | |
| cg11566441 | 11 | 1.12E+08 | + | OpenSea | DIXDC1; DIXDC1 | NM_033425; | Body; | 0 | DIXDC1 |
| | | | | | | NM_001037954 | Body | | |
| cg00242358 | 3 | 47619041 | − | N_Shore | CSPG5 | NM_006574 | Body | 0 | CSPG5 |
| cg15785704 | 17 | 30669010 | + | Island | C17orf75 | NM_022344 | Body | 0 | C17orf75 |
| cg09851545 | 8 | 72649950 | − | OpenSea | | | | 103825 | MSC |
| cg20899379 | 15 | 50839463 | + | OpenSea | USP50 | NM_203494 | TSS1500 | 560 | USP50 |
| cg01837362 | 12 | 34492938 | − | N_Shore | | | | 311701 | ALG10 |
| cg05498024 | 10 | 28917278 | − | OpenSea | | | | 5236 | WAC |
| cg12712184 | 7 | 1.56E+08 | − | OpenSea | | | | 185833 | SHH |
| cg26395211 | 5 | 1.4E+08 | − | Island | WDR55 | NM_017706 | TSS200 | 67 | WDR55 |
| cg12851609 | 18 | 24129562 | + | Island | KCTD1; KCTD1; | NM_198991; | 5′UTR; | 0 | KCTD1 |
| | | | | | KCTD1 | NM_001136205; | TSS200; | | |
| | | | | | | NM_001142730 | TSS1500 | | |
| cg01966129 | 22 | 22901880 | + | S_Shore | PRAME; | NM_206953; | TSS200; | 0 | LL22NC03- |
| | | | | | LOC648691; | NR_027426; | Body; | | 63E9.3 |
| | | | | | PRAME; PRAME; | NM_006115; | TSS200; | | |
| | | | | | PRAME; PRAME | NM_206955; | TSS1500; | | |
| | | | | | | NM_206956; | TSS1500; | | |
| | | | | | | NM_206954 | TSS200 | | |
| cg05592911 | 11 | 68722879 | − | OpenSea | | | | 14809 | IGHMBP2 |
| cg09436171 | 3 | 2142141 | + | S_Shore | CNTN4 | NM_175607 | TSS200 | 0 | CNTN4 |
| cg15822656 | 7 | 30321030 | − | N_Shelf | | | | 2891 | ZNRF2 |
| cg14074117 | 16 | 1909714 | + | OpenSea | C16orf73; | NM_152764; | Body; | 0 | MEIOB |
| | | | | | C16orf73 | NM_001163560 | Body | | |
| cg25323711 | 4 | 1.41E+08 | + | S_Shore | CLGN; CLGN | NM_001130675; | TSS1500; | 280 | CLGN |
| | | | | | | NM_004362 | TSS1500 | | |
| cg11813387 | 5 | 43310340 | − | N_Shelf | HMGCS1; | NM_002130; | 5′UTR; | 0 | HMGCS1 |
| | | | | | HMGCS1 | NM_001098272 | 5′UTR | | |
| cg13782176 | 10 | 6264761 | − | S_Shore | PFKFB3; PFKFB3 | NM_004566; | Body; | 0 | PFKFB3 |
| | | | | | | NM_001145443 | Body | | |
| cg17354052 | 16 | 23568699 | + | Island | UBFD1; EARS2; | NM_019116; | TSS200; | 2 | EARS2 |
| | | | | | EARS2 | NM_001083614; | TSS200; | | |
| | | | | | | NM_003501 | TSS200 | | |
| cg06938878 | 11 | 15094364 | − | N_Shore | CALCB | NM_000728 | TSS1500 | 780 | CALCB |
| cg09235723 | 11 | 65495317 | − | OpenSea | | | | 6907 | RNASEH2C |
| cg16980637 | 19 | 51815359 | − | Island | IGLON5 | NM_001101372 | Body | 0 | IGLON5 |
| cg17757376 | 7 | 91570087 | − | Island | AKAP9; AKAP9 | NM_147185; | TSS200; | 100 | AKAP9 |
| | | | | | | NM_005751 | TSS200 | | |
| cg14577211 | 12 | 48744457 | − | Island | ZNF641 | NM_152320 | TSS1500 | 0 | ZNF641 |
| cg23864993 | 4 | 8621266 | + | OpenSea | CPZ; CPZ; CPZ | NM_001014447; | Body; | 0 | CPZ |
| | | | | | | NM_003652; | Body; | | |
| | | | | | | NM_001014448 | Body | | |
| cg09622957 | 1 | 1.51E+08 | − | N_Shore | ADAMTSL4; | NM_019032; | TSS1500; | 706 | ADAMTSL4 |
| | | | | | ADAMTSL4 | NM_025008 | TSS1500 | | |
| cg27139805 | 19 | 35992791 | + | OpenSea | DMKN; DMKN; | NM_001126059; | Body; | 0 | DMKN |
| | | | | | DMKN; DMKN | NM_001035516; | 1stExon; | | |
| | | | | | | NM_001126061; | Body; | | |
| | | | | | | NM_033317 | Body | | |
| cg16880597 | 12 | 12420514 | + | Island | LRP6 | NM_002336 | TSS1500 | 702 | LRP6 |
| cg22684151 | 12 | 1.31E+08 | + | OpenSea | | | | 45506 | LQC100190940 |
| cg27477277 | 21 | 45811432 | − | Island | TRPM2 | NM_003307 | Body | 0 | TRPM2 |
| cg02451502 | 13 | 22486732 | + | OpenSea | | | | 34432 | LINC00424 |
| cg10052164 | 11 | 1750939 | − | OpenSea | HCCA2 | NM_053005 | Body | 0 | MOB2 |
| cg20853569 | 17 | 79247697 | − | OpenSea | SLC38A10; | NM_001037984; | Body; | 0 | SLC38A10 |
| | | | | | SLC38A10 | NM_138570 | Body | | |
| cg07695826 | 18 | 51884330 | − | Island | C18orf54 | NM_173529 | TSS1500 | 0 | C18orf54 |
| cg17859882 | 6 | 58287694 | − | Island | GUSBL2 | NM_003660 | Body | 0 | GUSBP4 |
| cg02305687 | 5 | 33937182 | − | Island | RXFP3 | NM_016568 | 1stExon | 0 | RXFP3 |
| cg19555331 | 4 | 1.48E+08 | + | Island | POU4F2 | NM_004575 | Body | 0 | POU4F2 |
| cg14025831 | 20 | 3873404 | + | S_Shelf | PANK2; PANK2; | NM_153638; | Body; | 0 | PANK2 |
| | | | | | PANK2 | NM_024960; | 5′UTR; | | |
| | | | | | | NM_153640 | 5′UTR | | |
| cg10682833 | 19 | 49614538 | + | N_Shelf | | | | 2667 | SNRNP70 |
| cg18816098 | 19 | 18335182 | − | Island | PDE4C; PDE4C; | NM_001098819; | 5′UTR; | 0 | PDE4C |
| | | | | | PDE4C; PDE4C | NM_001098818; | Body; | | |
| | | | | | | NM_001098819; | 1stExon; | | |
| | | | | | | NM_000923 | Body | | |
| cg08202287 | 2 | 2.32E+08 | − | N_Shore | B3GNT7 | NM_145236 | TSS1500 | 1052 | B3GNT7 |
| cg02953397 | 1 | 1238678 | + | N_Shelf | ACAP3 | NM_030649 | Body | 0 | ACAP3 |
| cg03585974 | 20 | 2800511 | + | N_Shore | | | | 2706 | TMEM239 |
| cg26282792 | 19 | 58554479 | − | Island | ZSCAN1 | NM_182572 | Body | 0 | ZSCAN1 |
| cg10436026 | 13 | 37453429 | − | OpenSea | SMAD9; SMAD9 | NM_001127217; | Body; | 0 | SMAD9 |
| | | | | | | NM_005905 | Body | | |
| cg09379188 | 8 | 59036394 | − | OpenSea | FAM110B | NM_147189 | 5′UTR | 0 | FAM110B |
| cg25097904 | 5 | 1.42E+08 | + | N_Shore | | | | 31474 | SPRY4 |
| cg01456989 | 11 | 61897937 | + | OpenSea | INCENP; INCENP | NM_020238; | Body; | 0 | INCENP |
| | | | | | | NM_001040694 | Body | | |
| cg06297356 | 16 | 1012735 | + | S_Shore | LMF1 | NM_022773 | Body | 0 | LMF1 |
| cg03521085 | 4 | 1219008 | + | Island | CTBP1; CTBP1 | NM_001328; | Body; | 0 | CTBP1 |
| | | | | | | NM_001012614 | Body | | |
| cg15524381 | 1 | 1.62E+08 | − | OpenSea | ATF6 | NM_007348 | Body | 0 | ATF6 |
| cg13573036 | 1 | 1.2E+08 | − | S_Shore | | | | 19671 | TBX15 |
| cg08704196 | 16 | 34787392 | − | Island | | | | 46551 | LOC100130700 |
| cg23670415 | 3 | 57583409 | − | Island | ARF4 | NM_001660 | TSS200 | 193 | ARF4 |
| cg14343652 | 7 | 1.31E+08 | − | N_Shore | FLJ43663; | NR_015431; | Body; | 0 | LINC-PINT |
| | | | | | FLJ43663 | NR_024153 | Body | | |
| cg09290175 | 22 | 39688265 | + | Island | | | | 20620 | RPL3 |
| cg11628781 | 5 | 1089021 | + | Island | SLC12A7 | NM_006598 | Body | 0 | SLC12A7 |
| cg01050736 | 6 | 32710583 | − | OpenSea | HLA-DQA2 | NM_020056 | Body | 0 | HLA-DQA2 |
| cg00519002 | 16 | 56659338 | − | Island | MT1E | NM_175617 | TSS1500 | 0 | MT1A |
| cg15784784 | 11 | 66628295 | − | S_Shore | PC; PC; PC | NM_022172; | Body; | 0 | PC |
| | | | | | | NM_000920; | Body; | | |
| | | | | | | NM_001040716 | Body | | |
| cg24048008 | 1 | 1.1E+08 | + | N_Shore | SCARNA2 | NM_003023 | TSS1500 | 472 | SCARNA2 |
| cg25877009 | 8 | 1.42E+08 | − | OpenSea | | | | 24291 | SLC45A4 |
| cg00181968 | 1 | 1.8E+08 | + | Island | FAM163A | NM_173509 | TSS200 | 0 | FAM163A |
| cg05872236 | 18 | 47825225 | + | Island | | | | 10532 | CXXC1 |
| cg11524248 | 5 | 1.5E+08 | + | N_Shore | CDX1 | NM_001804 | TSS1500 | 435 | CDX1 |
| cg18117780 | 17 | 66194407 | + | Island | LOC440461 | NR_027283 | TSS1500 | 392 | LOC440461 |
| cg12922648 | 6 | 30877739 | + | S_Shore | GTF2H4 | NM_001517 | Body | 0 | GTF2H4 |
| cg18259003 | 17 | 72347925 | − | Island | KIF19 | NM_153209 | Body | 0 | KIF19 |
| cg01763636 | 12 | 1.33E+08 | − | N_Shore | EP400NL | NR_003290 | TSS1500 | 261 | EP400NL |
| cg22715760 | 15 | 44069288 | + | Island | ELL3; ELL3 | NM_025165; | 5′UTR; | 0 | ELL3 |
| | | | | | | NM_025165 | 1stExon | | |
| cg18710162 | 17 | 33390808 | − | OpenSea | RFFL; RFFL | NM_001017368; | TSS200; | 0 | RAD51L3-RFFL |
| | | | | | | NM_057178 | 5′UTR | | |
| cg03347095 | 7 | 1.58E+08 | − | OpenSea | PTPRN2; PTPRN2; | NM_002847; | Body; | 0 | PTPRN2 |
| | | | | | PTPRN2 | NM_130842; | Body; | | |
| | | | | | | NM_130843 | Body | | |
| cg17232883 | 11 | 59318136 | − | OpenSea | | | | 23733 | OSBP |
| cg26373171 | 1 | 2.1E+08 | − | S_Shore | C1orf107 | NM_014388 | Body | 0 | DIEXF |
| cg05594095 | 1 | 6476916 | − | N_Shelf | HES2 | NM_019089 | 3′UTR | 0 | HES2 |
| cg26428054 | 12 | 49484058 | − | Island | DHH | NM_021044 | Body | 0 | DHH |
| cg05241015 | 2 | 1748876 | + | Island | PXDN | NM_012293 | TSS1500 | 584 | PXDN |
| cg22694153 | 1 | 46768912 | + | Island | LRRC41; UQCRH | NM_006369; | 1stExon; | 0 | LRRC41 |
| | | | | | | NM_006004 | TSS1500 | | |
| cg06765785 | 11 | 2020391 | + | S_Shore | H19 | NR_002196 | TSS1500 | 1325 | H19 |
| cg25593948 | 6 | 50786670 | + | N_Shore | TFAP2B | NM_003221 | 1stExon | 0 | TFAP2B |
| cg14162912 | 2 | 2.39E+08 | − | Island | LRRFIP1; LRRFIP1; | NM_001137550; | Body; | 0 | LRRFIP1 |
| | | | | | LRRFIP1; LRRFIP1; | NM_001137552; | TSS200; | | |
| | | | | | LRRFIP1 | NM_001137551; | TSS200; | | |
| | | | | | | NM_004735; | TSS200; | | |
| | | | | | | NM_001137553 | TSS200 | | |
| cg12463883 | 13 | 1.08E+08 | − | OpenSea | FAM155A | NM_001080396 | Body | 0 | FAM155A |
| cg16911220 | 12 | 52695728 | − | Island | KRT86 | NM_002284 | 1stExon | 0 | KRT86 |
| cg02917867 | 19 | 1265999 | + | Island | | | | 1469 | CIRBP-AS1 |
| cg03595533 | 6 | 28784788 | − | S_Shelf | | | | 42612 | LOC401242 |
| cg26589069 | 16 | 4983989 | − | N_Shelf | PPL | NM_002705 | Body | 0 | PPL |
| cg23996302 | 1 | 2.01E+08 | + | S_Shore | | | | 100 | TMEM9 |
| cg08033031 | 19 | 16528764 | − | Island | EPS15L1 | NM_021235 | Body | 0 | EPS15L1 |
| cg02792794 | 12 | 1.1E+08 | − | Island | ACACB | NM_001093 | Body | 0 | ACACB |
| cg21492942 | 20 | 44718714 | − | Island | NCOA5 | NM_020967 | TSS200 | 133 | NCOA5 |
| cg22876894 | 20 | 1783624 | − | N_Shore | | | | 23231 | LOC100289473 |
| cg16838749 | 2 | 2.41E+08 | + | S_Shore | | | | 112046 | GPC1 |
| cg22190114 | 19 | 56459234 | − | OpenSea | NLRP8; NLRP8 | NM_176811; | 5′UTR; | 0 | NLRP8 |
| | | | | | | NM_176811 | 1stExon | | |
| cg13341153 | 10 | 1.2E+08 | + | OpenSea | | | | 31377 | CASC2 |
| cg04430996 | 16 | 591437 | − | Island | SOLH | NM_005632 | 5′UTR | 0 | CARN15 |
| cg18862171 | 11 | 66314367 | − | Island | ACTN3; ZDHHC24 | NM_001104; | TSS200; | 0 | ACTN3 |
| | | | | | | NM_207340 | TSS1500 | | |
| cg01184449 | 2 | 42396170 | + | N_Shore | EML4; EML4 | NM_019063; | TSS1500; | 318 | EML4 |
| | | | | | | NM_001145076 | TSS1500 | | |
| cg14076923 | 9 | 1.24E+08 | + | OpenSea | GSN; GSN; GSN; | NM_198252; | Body; | 0 | GSN |
| | | | | | GSN; GSN; GSN; | NM_001127665; | Body; | | |
| | | | | | GSN; GSN | NM_001127667; | Body; | | |
| | | | | | | NM_001127664; | Body; | | |
| | | | | | | NM_001127663; | Body; | | |
| | | | | | | NM_000177; | Body; | | |
| | | | | | | NM_001127666; | Body; | | |
| | | | | | | NM_001127662 | Body | | |
| cg 14511156 | 19 | 54604124 | − | N_Shore | OSCAR; OSCAR; | NM_133168; | 5′UTR; | 0 | OSCAR |
| | | | | | OSCAR; OSCAR; | NM_206818; | 5′UTR; | | |
| | | | | | OSCAR; OSCAR; | NM_133169; | 5′UTR; | | |
| | | | | | OSCAR; OSCAR | NM_130771; | 1stExon; | | |
| | | | | | | NM_133169; | 1stExon; | | |
| | | | | | | NM_130771; | 5′UTR; | | |
| | | | | | | NM_133168; | 1stExon; | | |
| | | | | | | NM_206818 | 1stExon | | |
| cg21201109 | 19 | 11307090 | − | N_Shore | KANK2 | NM_001136191 | 5′UTR | 0 | KANK2 |
| cg22084428 | 3 | 98242050 | + | S_Shore | CLDND1; CLDND1; | NM_001040183; | TSS200; | 139 | CLDND1 |
| | | | | | CLDND1; CLDND1; | NM_019895; | TSS200; | | |
| | | | | | CLDND1; CLDND1 | NM_001040182; | TSS200; | | |
| | | | | | | NM_001040181; | TSS200; | | |
| | | | | | | NM_001040199; | TSS200; | | |
| | | | | | | NM_001040200 | TSS200 | | |
| cg02230254 | 11 | 65586170 | − | S_Shore | | | | 15238 | SNX32 |
| cg03420242 | 16 | 49893122 | − | Island | | | | 1291 | ZNF423 |
| cg16451027 | 5 | 1.12E+08 | − | N_Shore | APC | NM_001127511 | TSS1500 | 341 | APC |
| cg01405445 | 19 | 36246406 | − | Island | HSPB6 | NM_144617 | 3′UTR | 0 | HSPB6 |
| cg19623237 | 17 | 77818582 | + | Island | | | | 5368 | CBX4 |
| cg23483572 | 15 | 55879749 | − | N_Shore | PYGO1 | NM_015617 | Body | 0 | PYGO1 |
| cg21253590 | 7 | 1.02E+08 | − | N_Shore | ORAI2; ORAI2 | NM_001126340; | TSS1500; | 855 | ORAI2 |
| | | | | | | NM_032831 | TSS1500 | | |
| cg02159111 | 2 | 2.39E+08 | − | N_Shore | | | | 596 | ASB1 |
| cg25906638 | 10 | 10914374 | + | OpenSea | | | | 62527 | LINC00710 |
| cg20303995 | 10 | 1.04E+08 | − | Island | NFKB2; NFKB2; | NM_001077494; | Body; | 0 | NFKB2 |
| | | | | | NFKB2 | NM_001077493; | Body; | | |
| | | | | | | NM_002502 | Body | | |
| cg19571004 | 10 | 1.35E+08 | − | N_Shore | CYP2E1 | NM_000773 | TSS200 | 1.5 | CYP2E1 |
| cg18961589 | 10 | 1.34E+08 | + | OpenSea | | | | 6063 | LINC01164 |
| cg17794169 | 10 | 1.23E+08 | + | Island | FGFR2; FGFR2; | NM_000141; | TSS200; | 40 | FGFR2 |
| | | | | | FGFR2; FGFR2; | NM_001144917; | TSS200; | | |
| | | | | | FGFR2 | NM_001144919; | TSS200; | | |
| | | | | | | NM_022970; | TSS200; | | |
| | | | | | | NM_001144918 | TSS200 | | |
| cg23956119 | 1 | 2.28E+08 | + | Island | WNT9A | NM_003395 | Body | 0 | WNT9A |
| cg02847037 | 13 | 22033729 | + | Island | ZDHHC20 | NM_153251 | TSS1500 | 220 | ZDHHC20 |
| cg02704931 | 15 | 57998412 | − | N_Shore | GCOM1; GRINL1A; | NM_001018090; | Body; | 0 | GCOM1 |
| | | | | | GRINL1A; GCOM1; | NM_015532; | TSS1500; | | |
| | | | | | GRINL1A | NM_001018102; | TSS1500; | | |
| | | | | | | NM_001018091; | Body; | | |
| | | | | | | NM_027390 | TSS1500 | | |
| cg11706080 | 2 | 12856970 | − | Island | TRIB2; TRIB2 | NM_021643; | TSS200; | 26 | TRIB2 |
| | | | | | | NM_027303 | TSS200 | | |
| cg04040861 | 8 | 1.45E+08 | + | OpenSea | ZC3H3 | NM_015117 | Body | 0 | ZC3H3 |
| cg10915739 | 21 | 34405733 | + | Island | | | | 4229 | OLIG2 |
| cg16038738 | 1 | 1.09E+08 | + | S_Shore | FAM102B | NM_001010883 | Body | 0 | FAM102B |
| cg20061722 | 1 | 2.33E+08 | − | N_Shore | KIAA1804 | NM_032435 | TSS200 | 20 | KIAA1804 |
| cg01048272 | 17 | 80169878 | − | N_Shore | CCDC57 | NM_198082 | 5′UTR | 0 | CCDC57 |
| cg15216078 | 10 | 1.35E+08 | + | S_Shore | INPP5A | NM_005539 | Body | 0 | INPP5A |
| cg01917669 | 21 | 47056698 | + | N_Shore | | | | 6983 | PCBPS |
| cg16924102 | 4 | 20044588 | − | OpenSea | | | | 210645 | SLIT2 |
| cg11165881 | 3 | 238048 | + | N_Shore | CHL1 | NM_006614 | TSS1500 | 229 | CHL1 |
| cg22406758 | 11 | 631684 | − | S_Shelf | | | | 4510 | SCT |
| cg23993005 | 10 | 91091209 | + | OpenSea | IFIT3; IFIT3 | NM_001549; | Body; | 0 | LIPA |
| | | | | | | NM_001031683 | TSS1500 | | |
| cg26329020 | 12 | 32909488 | + | S_Shore | YARS2 | NM_001040436 | TSS1500 | 600 | YARS2 |
| cg11835619 | 12 | 56475064 | − | S_Shore | ERBB3; ERBB3 | NM_001005915; | Body; | 0 | ERBB3 |
| | | | | | | NM_001982 | Body | | |
| cg02141498 | 16 | 86912611 | − | Island | | | | 297306 | FOXL1 |
| cg15616511 | 11 | 45887415 | + | OpenSea | CRY2; CRY2 | NM_001127457; | Body; | 0 | CRY2 |
| | | | | | | NM_021117 | Body | | |
| cg07802917 | 3 | 1.24E+08 | + | Island | MYLK; MYLK; | NM_053025; | TSS200; | 161 | MYLK |
| | | | | | MYLK; MYLK | NM_053026; | TSS200; | | |
| | | | | | | NM_053028; | TSS200; | | |
| | | | | | | NM_053027 | TSS200 | | |
| cg13627062 | 15 | 96888433 | − | N_Shore | | | | 4940 | NR2F2 |
| cg21249093 | 9 | 73737300 | + | OpenSea | TRPM3 | NM_001007471 | TSS1500 | 0 | TRPM3 |
| cg07657332 | 10 | 54643873 | + | OpenSea | | | | 112412 | MBL2 |
| cg05583103 | 19 | 36157607 | − | OpenSea | UPK1A | NM_007000 | TSS200 | 106 | UPK1A |
| cg15438794 | 11 | 1012101 | − | S_Shore | AP2A2 | NM_012305 | 3′UTR | 0 | AP2A2 |
| cg16352283 | 1 | 27338978 | + | Island | FAM46B | NM_052943 | 1stExon | 0 | FAM46B |
| cg08627624 | 10 | 70031114 | − | OpenSea | | | | 11301 | PBLD |
| cg13788515 | 2 | 2.07E+08 | − | Island | ZDBF2 | NM_020923 | TSS200 | 76 | ZDBF2 |
| cg09231416 | 1 | 5940136 | + | S_Shelf | NPHP4 | NM_015102 | Body | 0 | NPHP4 |
| cg09330551 | 19 | 51334432 | + | S_Shelf | KLK15; KLK15; | NM_017509; | Body; | 0 | KLK15 |
| | | | | | KLK15 | NM_138563; | Body; | | |
| | | | | | | NM_138S64 | Body | | |
| cg10290200 | 7 | 1.28E+08 | − | N_Shore | FLNC; FLNC | NM_001127487; | Body; | 0 | FLNC |
| | | | | | | NM_001458 | Body | | |
| cg09636202 | 7 | 56243297 | + | Island | | | | 59206 | NUPR1L |
| cg21248305 | 6 | 30132715 | + | OpenSea | TRIM15 | NM_033229 | Body | 0 | TRIM15 |
| cg12851717 | 13 | 1.01E+08 | − | OpenSea | CLYBL | NM_206808 | Body | 0 | CLYBL |
| cg08686879 | 5 | 1.31E+08 | + | OpenSea | CSF2 | NM_000758 | 1stExon | 0 | CSF2 |
| cg00293191 | 16 | 73126120 | − | OpenSea | HTA | NR_027756 | TSS200 | 126 | HCCAT5 |
| cg11177980 | 1 | 41982115 | − | S_Shore | HIVEP3; HIVEP3 | NM_024503; | Body; | 0 | HIVEP3 |
| | | | | | | NM_001127714 | Body | | |
| cg21479731 | 4 | 2464579 | − | Island | | | | 0 | CFAP99 |
| cg14016554 | 6 | 31696509 | + | Island | DDAH2 | NM_013974 | Body | 0 | DDAH2 |
| cg26697583 | 15 | 89992598 | − | OpenSea | | | | 22038 | RHCG |
| cg10858945 | 10 | 1.17E+08 | − | Island | | | | 298 | ABUM1 |
| cg17515155 | 1 | 11714790 | − | Island | FBXO2; FBXO44; | NM_012168; | TSS200; | 0 | FBXO2 |
| | | | | | FBXO44; FBXO44; | NM_183413; | TSS200; | | |
| | | | | | FBXO44 | NM_001014765; | TSS200; | | |
| | | | | | | NM_033182; | TSS200; | | |
| | | | | | | NM_183412 | 5′UTR | | |
| cg06980252 | 7 | 1.38E+08 | + | OpenSea | CREB3L2 | NM_194071 | Body | 0 | CREB3L2 |
| cg06076923 | 1 | 54663141 | + | N_Shelf | CYB5RL | NM_001031672 | 5′UTR | 0 | CYB5RL |
| cg13966883 | 4 | 1.86E+08 | + | Island | HELT | NM_001029887 | Body | 0 | HELT |
| cg19324027 | 2 | 74699582 | − | Island | MRPL53 | NM_053050 | Body | 0 | CCDC142 |
| cg03561416 | 6 | 1.57E+08 | + | OpenSea | ARID1B; ARID1B; | NM_317519; | Body; | 0 | ARID1B |
| | | | | | ARID1B | NM_175863; | Body; | | |
| | | | | | | NM_320732 | Body | | |
| cg21211413 | 15 | 89348748 | − | S_Shore | ACAN; ACAN | NM_013227; | 5′UTR; | 0 | ACAN |
| | | | | | | NM_001135 | 5′UTR | | |
| cg09760644 | 6 | 1.67E+08 | − | N_Shelf | | | | 611 | RPS6KA2 |
| cg06287140 | 12 | 1.22E+08 | + | OpenSea | BCL7A; BCL7A | NM_001024808; | Body; | 0 | BCL7A |
| | | | | | | NM_020993 | Body | | |
| cg02930078 | 6 | 17706856 | + | Island | NUP153 | NM_005124 | TSS200 | 0 | NUP153 |
| cg03565674 | 11 | 1507321 | − | OpenSea | HCCA2 | NM_053005 | Body | 0 | M0B2 |
| cg16142349 | 6 | 1.67E+08 | − | OpenSea | RPS6KA2; | NM_021135; | Body; | 0 | RPS6KA2 |
| | | | | | RPS6KA2 | NM_001006932 | Body | | |
| cg12594244 | 15 | 74218754 | + | Island | LOXL1 | NM_005576 | TSS200 | 0 | LOXL1-AS1 |
| cg14777056 | 1 | 1.1E+08 | − | S_Shelf | | | | 1408 | C1orf194 |
| cg26997090 | 11 | 33682676 | − | S_Shelf | C11orf41 | NM_012194 | Body | 0 | KIAA1549L |
| cg06525670 | 13 | 30528708 | + | OpenSea | | | | 4082 | LINC00544 |
| cg25426716 | 5 | 1.26E+08 | + | OpenSea | GRAMD3 | NM_001146319 | TSS200 | 87 | GRAMD3 |
| cg05012661 | 4 | 6891264 | − | OpenSea | | | | 5364 | KIAA0232 |
| cg20490900 | 1 | 1502606 | + | OpenSea | SSU72 | NM_014188 | Body | 0 | SSU72 |
| cg00467202 | 16 | 30759573 | + | Island | PHKG2 | NM_000294 | TSS200 | 45 | PHKG2 |
| cg09326529 | 1 | 94528115 | + | OpenSea | ABCA4 | NM_000350 | Body | 0 | ABCA4 |
| cg20645601 | 7 | 2742371 | + | Island | AMZ1 | NM_133463 | Body | 0 | AMZ1 |
| cg07592012 | 17 | 79977307 | + | N_Shelf | STRA13 | NM_144998 | Body | 0 | STRA13 |
| cg09434085 | 7 | 1.57E+08 | + | Island | PTPRN2; PTPRN2; | NM_002847; | Body; | 0 | PTPRN2 |
| | | | | | PTPRN2 | NM_130842; | Body; | | |
| | | | | | | NM_130843 | Body | | |
| cg08895260 | 11 | 82756563 | − | OpenSea | RAB30 | NM_014488 | 5′UTR | 0 | RAB30 |
| cg02253142 | 15 | 52048211 | + | S_Shelf | TMOD2; TMOD2 | NM_014548; | 5′UTR; | 0 | TMOD2 |
| | | | | | | NM_001142885 | 5′UTR | | |
| cg12428906 | 11 | 18034757 | − | Island | SERGEF | NM_012139 | TSS200 | 119 | SERGEF |
| cg19454239 | 13 | 28430925 | + | OpenSea | | | | 62835 | GSX1 |
| cg19789047 | 5 | 1.42E+08 | + | OpenSea | | | | 30243 | SPRY4 |
| cg02392228 | 19 | 4523345 | + | N_Shore | PLIN5 | NM_001013706 | 3′UTR | 0 | PLINS |
| cg04177091 | 16 | 1030166 | + | Island | | | | 0 | LMF1 |
| cg08791849 | 13 | 42846648 | − | Island | AKAR11 | NM_016248 | 5′UTR | 0 | AKAP11 |
| cg1l902379 | 19 | 7681598 | − | N_Shore | KIAA1543; | NM_020902; | Body; | 0 | CAMSAP3 |
| | | | | | KIAA1543 | NM_001080429 | Body | | |
| cg09928766 | 19 | 51815373 | − | Island | IGLON5 | NM_001101372 | Body | 0 | IGLON5 |
| cg05158074 | 12 | 1.11E+08 | − | OpenSea | CCDC63 | NM_152591 | TSS1500 | 224 | CCDC63 |
| cg09505233 | 10 | 1.34E+08 | + | OpenSea | | | | 14651 | BNIP3 |
| cg00223950 | 1 | 19536222 | + | Island | UBR4 | NM_020765 | Body | 0 | UBR4 |
| cg02336461 | 17 | 57697246 | − | Island | CLTC; CLTC | NM_004859; | 1stExon; | 0 | CLTC |
| | | | | | | NM_004859 | 5′UTR | | |
| cg09266113 | 1 | 54356345 | − | S_Shore | YIPF1 | NM_018982 | TSS1500 | 857 | YIPF1 |
| cg05384646 | 15 | 30206862 | + | OpenSea | | | | 92155 | TJP1 |
| cg08211853 | 2 | 3658933 | + | OpenSea | COLEC11; | NM_024027; | Body; | 0 | COLEC11 |
| | | | | | COLEC11 | NM_99235 | Body | | |
| cg22226904 | 1 | 27819060 | − | S_Shelf | | | | 2381 | WASF2 |
| cg24435747 | 1 | 24514557 | − | S_Shore | IL28RA; IL28RA; | NM_173065; | TSS1500; | 791 | IFNLR1 |
| | | | | | IL28RA | NM_173064; | TSS1500; | | |
| | | | | | | NM_170743 | TSS1500 | | |
| cg08909157 | 9 | 215561 | − | S_Shore | DOCK8; C9orf66; | NM_203447; | Body; | 0 | C9orf66 |
| | | | | | C9orf66 | NM_152569; | 1stExon; | | |
| | | | | | | NM_152569 | 5′UTR | | |
| cg01863682 | 2 | 1.83E+08 | − | N_Shelf | NEURODI | NM_002500 | TSS1500 | 378 | CERKL |
| cg07664272 | 4 | 1.39E+08 | + | OpenSea | | | | 173746 | LINC00616 |
| cg12118784 | 19 | 47141551 | − | N_Shore | | | | 3611 | GNG8 |
| cg17972631 | 1 | 9970653 | − | Island | CTNNBIP1; | NM_001012329; | TSS1500; | 336 | CTNNBIP1 |
| | | | | | CTNNBIP1 | NM_020248 | TSS1500 | | |
| cg22681344 | 19 | 585501 | + | Island | | | | 2007 | BSG |
| cg27574066 | 17 | 26687382 | + | S_Shelf | TMEM199 | NM_152464 | Body | 0 | SARM1 |
| cg06138505 | 3 | 1.27E+08 | + | Island | MCM2 | NM_004526 | TSS200 | 0 | MCM2 |
| cg16103681 | 8 | 42752335 | − | Island | HOOK3; RNF170; | NM_032410; | Body; | 0 | HOOK3 |
| | | | | | RNF170; RNF170; | NR_027669; | TSS1500; | | |
| | | | | | RNF170; RNF170; | NM_001160224; | TSS1500; | | |
| | | | | | RNF170 | NM_001160223; | TSS1500; | | |
| | | | | | | NM_030954; | TSS1500; | | |
| | | | | | | NR_027668; | TSS1500; | | |
| | | | | | | NM_001160225 | TSS1500 | | |
| cg20487932 | 12 | 1.09E+08 | − | OpenSea | SSH1; SSH1 | NM_001161330; | Body; | 0 | SSH1 |
| | | | | | | NM_018984 | Body | | |
| cg23703963 | 14 | 1.06E+08 | − | Island | CRIP1; CRIP1 | NM_001311; | 1stExon; | 0 | CRIP1 |
| | | | | | | NM_001311 | 5′UTR | | |
| cg02492791 | 12 | 85306548 | − | Island | SLC6A15; | NM_001146335; | 1stExon; | 0 | SLC6A15 |
| | | | | | SLC6A15; | NM_001146335; | 5′UTR; | | |
| | | | | | SLC6A15; | NM_018057; | 5′UTR; | | |
| | | | | | SLC6A15; | NM_018057; | 1stExon; | | |
| | | | | | SLC6A15; SLC6A15 | NM_182767; | 1stExon; | | |
| | | | | | | NM_182767 | 5′UTR | | |
| cg05D19671 | 5 | 77943786 | + | N_Shore | LHFPL2 | NM_005779 | 5′UTR | 0 | LHFPL2 |
| cg19926434 | 14 | 66975296 | − | Island | GPHN; GPHN | NM_020806; | 1stExon; | 0 | GPHN |
| | | | | | | NM_001024218 | 1stExon | | |
| cg19398269 | 6 | 28678460 | + | OpenSea | | | | 123347 | ZBED9 |
| cg01395533 | 3 | 66024640 | + | Island | MAGI1; MAGI1; | NM_004742; | TSS200; | 130 | MAGI1 |
| | | | | | MAGI1 | NM_015520; | TSS200; | | |
| | | | | | | NM_001033057 | TSS200 | | |
| cg25652859 | 20 | 57427412 | − | N_Shore | GNAS; GNAS; | NM_080425; | TSS1500; | 0 | GNAS |
| | | | | | GNAS; GNASAS | NM_001077490; | TSS1500; | | |
| | | | | | | NM_016592; | 3′UTR; | | |
| | | | | | | NR_002785 | TSS1500 | | |
| cg00067600 | 3 | 34109141 | − | OpenSea | | | | 197941 | PDCD6IP |
| cg13978156 | 4 | 1.83E+08 | + | OpenSea | | | | 0 | TENM3 |
| cg20158944 | 2 | 2.43E+08 | − | OpenSea | | | | 79822 | RTP5 |
| cg07679834 | 19 | 19841125 | + | N_Shelf | ZNF14 | NM_021030 | Body | 0 | ZNF14 |
| cg10140536 | 19 | 57630596 | + | Island | USP29 | NM_020903 | TSS1500 | 911 | USP29 |
| cg12346874 | 2 | 1.38E+08 | − | Island | | | | 0 | THSD7B |
| cg11558474 | 9 | 74382743 | + | N_Shore | TMEM2; TMEM2 | NM_013390; | 5′UTR; | 0 | TMEM2 |
| | | | | | | NM_001135820 | 5′UTR | | |
| cg13629887 | 12 | 12876682 | + | Island | | | | 1376 | CDKN1B |
| cg24997589 | 7 | 1.49E+08 | + | OpenSea | | | | 20039 | PDIA4 |
| cg06289444 | 6 | 1.7E+08 | + | N_Shore | THBS2 | NM_003247 | Body | 0 | THBS2 |
| cg22267234 | 11 | 9087524 | − | OpenSea | SCUBE2; SCUBE2 | NM_020974; | Body; | 0 | SCUBE2 |
| | | | | | | NM_001170690 | Body | | |
| cg23840481 | 16 | 4666699 | − | Island | | | | 1771 | UBALD1 |
| cg24879415 | 17 | 74534700 | − | S_Shore | CYGB; PRCD | NM_134268; | TSS1500; | 0 | PRCD |
| | | | | | | NM_001077620 | TSS1500 | | |
| cg14220544 | 16 | 89627035 | + | Island | RPL13; RPL13; | NM_033251; | TSS200; | 28 | RPL13 |
| | | | | | SNORD68 | NM_000977; | TSS200; | | |
| | | | | | | NM_002450 | TSS1500 | | |
| cg10984252 | 10 | 1.15E+08 | − | Island | CASP7; CASP7; | NM_033340; | 5′UTR; | 0 | CASP7 |
| | | | | | CASP7; CASP7 | NM_033338; | TSS200; | | |
| | | | | | | NM_001227; | TSS200; | | |
| | | | | | | NM_033339 | TSS200 | | |
| cg10536462 | 4 | 870491 | − | N_Shore | GAK | NM_005255 | Body | 0 | GAK |
| cg16782174 | 2 | 1.28E+08 | − | Island | LIMS2; LIMS2; | NM_001136037; | Body; | 0 | LIMS2 |
| | | | | | LIMS2; LIMS2 | NM_017980; | Body; | | |
| | | | | | | NM_001161403; | Body; | | |
| | | | | | | NM_001161404 | Body | | |
| cg02797036 | 7 | 44084886 | − | S_Shore | DBNL; DBNL; DBNL | NM_001122956; | Body; | 0 | DBNL |
| | | | | | | NM_001014436; | Body; | | |
| | | | | | | NM_014063 | Body | | |
| cg21355487 | 10 | 49688893 | − | S_Shore | ARHGAP22 | NM_021226 | Body | 0 | ARHGAP22 |
| cg23186422 | 6 | 1.39E+08 | − | Island | | | | 0 | FLJ46906 |
| cg23334306 | 14 | 33945217 | − | OpenSea | NPAS3; NPAS3; | NM_001164749; | Body; | 0 | NPAS3 |
| | | | | | NPAS3; NPAS3 | NM_173159; | Body; | | |
| | | | | | | NM_001165893; | Body; | | |
| | | | | | | NM_022123 | Body | | |
| cg10985158 | 1 | 2.02E+08 | − | OpenSea | NAV1 | NM_020443 | Body | 0 | NAV1 |
| cg19312085 | 1 | 2.48E+08 | − | OpenSea | | | | 25620 | NLRP3 |
| cg12039242 | 8 | 1.02E+08 | + | Island | YWHAZ; YWHAZ; | NM_001135701; | 5′UTR; | 0 | YWHAZ |
| | | | | | YWHAZ; YWHAZ; | NM_003406; | 5′UTR; | | |
| | | | | | YWHAZ; YWHAZ | NM_145690; | 5′UTR; | | |
| | | | | | | NM_001135700; | 5′UTR; | | |
| | | | | | | NM_001135699; | 5′UTR; | | |
| | | | | | | NM_001135702 | TSS200 | | |
| cg25233139 | 13 | 1.12E+08 | + | OpenSea | | | | 190801 | TEX29 |
| cg15126179 | 13 | 79181338 | + | Island | | | | 0 | RNF219-AS1 |
| cg11915641 | 2 | 1.03E+08 | + | Island | SLC9A2 | NM_003048 | 1stExon | 0 | SLC9A2 |
| cg11971789 | 19 | 42545623 | − | N_Shore | GRIK5 | NM_002088 | Body | 0 | GRIK5 |
| cg20933713 | 1 | 1.76E+08 | + | OpenSea | TNR | NM_003285 | TSS1500 | 466 | TNR |
| cg16390249 | 7 | 2138435 | − | OpenSea | MAD1L1; | NM_003550; | Body; | 0 | MAD1L1 |
| | | | | | MAD1L1; MAD1L1 | NM_001013837; | Body; | | |
| | | | | | | NM_001013836 | Body | | |
| cg25135084 | 7 | 1.49E+08 | − | Island | ZNF425 | NM_001001661 | Body | 0 | ZNF425 |
| cg07470512 | 20 | 32255052 | − | Island | NECAB3; NECAB3; | NM_031231; | Body; | 0 | NECAB3 |
| | | | | | C20orf134; | NM_031232; | Body; | | |
| | | | | | C20orf134 | NM_001024675; | 1stExon; | | |
| | | | | | | NM_001024675 | 5′UTR | | |
| cg25783497 | 1 | 3307070 | − | Island | PRDM16; PRDM16 | NM_022114; | Body; | 0 | PRDM16 |
| | | | | | | NM_199454 | Body | | |
| cg19734801 | 5 | 1.31E+08 | − | Island | | | | 0 | ACSL6 |
| cg04949193 | 6 | 1.57E+08 | + | OpenSea | ARID1B; ARID1B; | NM_017519; | Body; | 0 | ARID1B |
| | | | | | ARID1B | NM_175863; | Body; | | |
| | | | | | | NM_020732 | Body | | |
| cg18663063 | 5 | 68710907 | − | Island | MARVELD2; | NM_001038603; | TSS200; | 30 | MARVELD2 |
| | | | | | MARVELD2 | NM_144724 | TSS200 | | |
| cg26953232 | 6 | 32942494 | − | S_Shore | BRD2; BRD2 | NM_001113182; | Body; | 0 | BRD2 |
| | | | | | | NM_005104 | Body | | |
| cg15685943 | 1 | 1.16E+08 | − | Island | TSPAN2 | NM_005725 | TSS200 | 150 | TSPAN2 |
| cg01915076 | 12 | 49393228 | + | Island | DDN | NM_015086 | TSS200 | 139 | DDN |
| cg12414557 | 4 | 1107495 | − | Island | RNF212; RNF212; | NM_001131034; | 1stExon; | 142 | RNF212 |
| | | | | | RNF212; RNF212 | NM_001131034; | 5′UTR; | | |
| | | | | | | NM_194439; | 1stExon; | | |
| | | | | | | NM_194439 | 5′UTR | | |
| cg05715138 | 17 | 4534892 | + | OpenSea | ALOX15 | NM_001140 | 3′UTR | 0 | ALOX15 |
| cg04479212 | 3 | 37495166 | + | S_Shore | ITGA9 | NM_002207 | Body | 0 | ITGA9 |
| cg11591485 | 3 | 1.25E+08 | + | OpenSea | SLC12A8 | NM_024628 | Body | 0 | SLC12A8 |
| cg24202936 | 11 | 50257256 | − | Island | LOC441601 | NM_003034 | Body | 0 | LOC441601 |
| cg12373573 | 12 | 85430025 | − | OpenSea | TSPAN19; | NM_001100917; | 1stExon; | 0 | TSPAN19 |
| | | | | | TSPAN19; LRRIQ1; | NM_001100917; | 5′UTR; | | |
| | | | | | LRRIQ1 | NM_001079910; | TSS200; | | |
| | | | | | | NM_032165 | TSS200 | | |
| cg18789261 | 12 | 82752393 | + | Island | C12orf26; | NM_032230; | 1stExon; | 0 | CCDC59 |
| | | | | | CCDC59; CCDC59 | NM_033192; | Body; | | |
| | | | | | | NM_014167 | TSS200 | | |
| cg09075268 | 16 | 1270372 | − | S_Shore | CACNA1H; | NM_021098; | Body; | 0 | CACNA1H |
| | | | | | CACNA1H | NM_001005407 | Body | | |
| cg17802216 | 6 | 1.69E+08 | − | OpenSea | SMOC2; SMOC2 | NM_022138; | Body; | 0 | SMOC2 |
| | | | | | | NM_001166412 | Body | | |
| cg09962377 | 16 | 522007 | − | OpenSea | RAB11FIP3 | NM_014700 | Body | 0 | RAB11FIP3 |
| cg23912217 | 10 | 81839159 | − | S_Shore | LOC219347; | NR_027430; | TSS1500; | 0 | TMEM254 |
| | | | | | LOC219347; | NR_027431; | TSS1500; | | |
| | | | | | LOC219347; | NM_027428; | TSS1500; | | |
| | | | | | C10orf57; | NM_025125; | Body; | | |
| | | | | | LOC219347; | NR_027432; | TSS1500; | | |
| | | | | | LOC219347 | NR_027429 | TSS1500 | | |
| cg14966562 | 5 | 32444709 | + | Island | ZFR | NM_016107 | Body | 0 | ZFR |
| cg18512446 | 18 | 77192641 | − | N_Shore | NFATC1; NFATC1; | NM_006162; | Body; | 0 | NFATC1 |
| | | | | | NFATC1; NFATC1; | NM_172388; | 5′UTR; | | |
| | | | | | NFATC1 | NM_172389; | Body; | | |
| | | | | | | NM_172387; | Body; | | |
| | | | | | | NM_172390 | Body | | |
| cg05481257 | 2 | 20870211 | − | Island | GDF7 | NM_182828 | Body | 0 | GDF7 |
| cg04444415 | 3 | 1.93E+08 | + | OpenSea | ATP13A4 | NM_032279 | TSS200 | 172 | ATP13A4 |
| cg09440812 | 11 | 66610597 | − | Island | RCE1; RCE1; | NM_005133; | TSS1500; | 0 | C11orf80 |
| | | | | | C11orf80 | NM_001032279; | TSS1500; | | |
| | | | | | | NM_024650 | Body | | |
| cg05232576 | 20 | 2777921 | − | N_Shelf | CPXM1 | NM_019609 | Body | 0 | CPXM1 |
| cg05250061 | 4 | 1.84E+08 | + | OpenSea | WWC2 | NM_024949 | Body | 0 | WWC2 |
| cg22606129 | 7 | 33714254 | + | OpenSea | | | | 68573 | BBS9 |
| cg26319800 | 2 | 74570700 | + | OpenSea | SLC4A5 | NM_133478 | TSS200 | 0 | SLC4A5 |
| cg02627599 | 6 | 1.49E+08 | + | N_Shore | | | | 7415 | SASH1 |
| cg22776392 | 1 | 1.81E+08 | + | Island | | | | 3646 | KIAA1614 |
| cg03051116 | 10 | 30720703 | − | N_Shore | | | | 2245 | MAP3K8 |
| cg20318252 | 17 | 55594714 | + | OpenSea | MSI2; MSI2 | NM_138962; | Body; | 0 | MSI2 |
| | | | | | | NM_170721 | Body | | |
| cg03933810 | 17 | 4634545 | − | N_Shore | MED11 | NM_001001683 | TSS200 | 176 | MED11 |
| cg10318443 | 17 | 685449 | + | Island | GLOD4; RNMTL1 | NM_016080; | 1stExon; | 0 | GLOD4 |
| | | | | | | NM_018146 | TSS200 | | |
| cg00859655 | 11 | 7694524 | + | N_Shore | CYB5R2 | NM_016229 | 5′UTR | 0 | CYB5R2 |
| cg12798700 | 6 | 1.26E+08 | + | Island | HDDC2 | NM_016063 | Body | 0 | HDDC2 |
| cg17552093 | 17 | 79875985 | + | Island | SIRT7 | NM_016538 | 1stExon | 0 | SIRT7 |
| cg04267807 | 7 | 83278462 | + | OpenSea | SEMA3E | NM_012431 | TSS200 | 0 | SEMA3E |
| cg19905880 | 11 | 67049324 | + | N_Shelf | ADRBK1 | NM_001619 | Body | 0 | ADRBK1 |
| cg20316284 | 22 | 38794946 | + | Island | LOC400927 | NM_002821 | TSS200 | 14 | LOC400927 |
| cg26945050 | 2 | 27346434 | − | Island | ABHD1 | NM_032604 | TSS1500 | 221 | ABHD1 |
| cg19788371 | 8 | 1.43E+08 | + | OpenSea | | | | 171400 | MIR4472-1 |
| cg02601685 | 17 | 55055297 | − | Island | SCPEP1 | NM_021626 | TSS200 | 169 | SCPEP1 |
| cg21306321 | 19 | 869024 | − | N_Shore | MED16 | NM_005481 | Body | 0 | MED16 |
| cg10194829 | 8 | 21900177 | + | OpenSea | FGF17 | NM_003867 | TSS1500 | 249 | FGF17 |
| cg01011367 | 10 | 1.25E+08 | + | OpenSea | ACADSB | NM_001609 | Body | 0 | ACADSB |
| cg08168844 | 16 | 29830973 | + | S_Shelf | C16orf53; MVP; | NM_024516; | Body; | 0 | BOLA2 |
| | | | | | MVP | NM_017458; | TSS1500; | | |
| | | | | | | NM_005115 | TSS1500 | | |
| cg26350143 | 11 | 2985721 | + | OpenSea | SNORA54; NAP1L4 | NR_002982; | TSS1500; | 0 | NAP1L4 |
| | | | | | | NM_005969 | Body | | |
| cg21635706 | 15 | 69432565 | − | OpenSea | MIR548H4 | NM_031680 | Body | 0 | MIR548H4 |
| cg12817908 | 3 | 1.39E+08 | − | S_Shore | NMNAT3 | NM_178177 | TSS1500 | 293 | NMNAT3 |
| cg06046805 | 5 | 58299024 | − | OpenSea | PDE4D; PDE4D; | NM_001165899; | Body; | 0 | PDE4D |
| | | | | | PDE4D | NM_001104631; | Body; | | |
| | | | | | | NM_006203 | Body | | |
| cg11091113 | 14 | 37133168 | − | S_Shore | PAX9 | NM_006194 | Body | 0 | PAX9 |
| cg07327178 | 5 | 8457729 | + | Island | | | | 51 | LOC729506 |
| cg26758396 | 20 | 6104274 | + | Sphere | FERMT1 | NM_017671 | TSS200 | 82 | FERMT1 |
| cg11710912 | 13 | 1.07E+08 | − | N_Shelf | ARGLUI | NM_018011 | Body | 0 | ARGLU1 |
| cg17413194 | 17 | 63053996 | + | S_Shore | GNA13 | NM_006572 | TSS1500 | 1075 | GNA13 |
| cg08324950 | 19 | 1315962 | + | Island | | | | 14532 | EFNA2 |
| cg17315247 | 7 | 2754218 | − | N_Shelf | AMZ1 | NM_133463 | 3′UTR | 0 | AMZ1 |
| cg10149337 | 18 | 76734278 | + | Island | | | | 5995 | SALL3 |
| cg03296248 | 13 | 1.11E+08 | + | Island | COL4A1 | NM_001845 | Body | 0 | COL4A1 |
| cg17093877 | 17 | 72206412 | + | N_Shelf | MGC16275 | NR_026914 | Body | 0 | MGC16275 |
| cg05263760 | 6 | 12845743 | − | OpenSea | PHACTR1 | NM_030948 | Body | 0 | PHACTR1 |
| cg24819967 | 10 | 1.35E+08 | − | N_Shore | | | | 1879 | PAOX |
| cg21492308 | 2 | 3750128 | − | N_Shore | ALLC | NM_018436 | Body | 0 | ALLC |
| cg13092108 | 1 | 26857284 | − | S_Shore | RPS6KA1 | NM_002953 | Body | 0 | RPS6KA1 |
| cg03422070 | 11 | 2722082 | + | Island | KCNQ1OT1; | NR_002728; | TSS1500; | 0 | KCNQ1 |
| | | | | | KCNQ1; KCNQ1 | NM_000218; | Body; | | |
| | | | | | | NM_181798 | Body | | |
| cg19233472 | 5 | 1.7E+08 | − | N_Shore | FOXI1; FOXI1 | NM_144769; | TSS200; | 163 | FOXI1 |
| | | | | | | NM_012188 | TSS200 | | |
| cg27648567 | 13 | 37393923 | − | Island | RFXAP | NM_000538 | 1stExon | 0 | RFXAP |
| cg25484139 | 3 | 13008273 | + | N_Shore | IQSEC1; IQSEC1 | NM_001134382; | Body; | 0 | IQSEC1 |
| | | | | | | NM_014869 | Body | | |
| cg21768956 | 17 | 927652 | − | S_Shore | ABR; ABR; ABR | NM_021962; | Body; | 0 | ABR |
| | | | | | | NM_001092; | Body; | | |
| | | | | | | NM_001159746 | Body | | |
| cg13707224 | 9 | 1043825 | − | N_Shore | | | | 6519 | DMRT2 |
| cg18386828 | 11 | 1860235 | + | OpenSea | TNNI2; TNNI2; | NM_001145841; | TSS1500; | 0 | TNNI2 |
| | | | | | TNNI2; TNNI2 | NM_001145829; | TSS1500; | | |
| | | | | | | NM_003282; | 1stExon; | | |
| | | | | | | NM_003282 | 5′UTR | | |
| cg20349305 | 11 | 75919698 | + | Island | | | | 0 | WNT11 |
| cg13357482 | 6 | 1.06E+08 | + | Sphere | POPDC3 | NM_022361 | TSS200 | 158 | POPDC3 |
| cg00367396 | 6 | 42694557 | − | N_Shore | | | | 755 | ATP6V0CP3 |
| cg16588163 | 3 | 1.9E+08 | − | OpenSea | IL1RAP; IL1RAP; | NM_001167929; | 5′UTR; | 0 | IL1RAP |
| | | | | | IL1RAP; IL1RAP; | NM_001167931; | 5′UTR; | | |
| | | | | | IL1RAP; IL1RAP | NM_001167930; | 5′UTR; | | |
| | | | | | | NM_001167928; | 5′UTR; | | |
| | | | | | | NM_134470; | 5′UTR; | | |
| | | | | | | NM_002182 | 5′UTR | | |
| cg05755354 | 10 | 14372596 | − | OpenSea | FRMD4A; FRMD4A | NM_018027; | 1stExon; | 0 | FRMD4A |
| | | | | | | NM_018027 | 5′UTR | | |
| cg12157281 | 2 | 8168246 | + | Island | | | | 0 | LINC00299 |
| cg06940110 | 2 | 1.19E+08 | − | N_Shore | | | | 26476 | DDX18 |
| cg21969516 | 4 | 1.28E+08 | + | OpenSea | | | | 676329 | INTU |
| cg16673904 | 17 | 7521996 | − | S_Shelf | SHBG | NM_027463 | Body | 0 | SHBG |
| cg23442198 | 4 | 1.87E+08 | + | OpenSea | CYP4V2 | NM_207352 | Body | 0 | CYP4V2 |
| cg01678799 | 15 | 43481741 | + | S_Shelf | CCNDBP1; | NM_012142; | Body; | 0 | CCNDBP1 |
| | | | | | CCNDBP1; | NR_027513; | Body; | | |
| | | | | | CCNDBP1; | NR_027514; | Body; | | |
| | | | | | CCNDBP1 | NM_037370 | 5′UTR | | |
| cg18371471 | 14 | 1.01E+08 | − | OpenSea | BEGAIN; BEGAIN | NM_001159531; | Body; | 0 | BEGAIN |
| | | | | | | NM_020836 | Body | | |
| cg25976094 | 1 | 1.09E+08 | + | OpenSea | FAM102B | NM_001010883 | 3′UTR | 0 | FAM102B |
| cg18965213 | 15 | 62989995 | + | Island | TLN2 | NM_015059 | Body | 0 | TLN2 |
| cg06018240 | 7 | 1.51E+08 | − | OpenSea | SLC4A2 | NM_003040 | Body | 0 | SLC4A2 |
| cg18236982 | 1 | 2.36E+08 | − | OpenSea | | | | 18282 | LYST |
| cg06395280 | 2 | 2.42E+08 | − | Island | FARP2 | NM_014808 | TSS200 | 54 | FARP2 |
| cg07337446 | 6 | 46517898 | + | OpenSea | CYP39A1 | NM_016593 | 3′UTR | 0 | CYP39A1 |
| cg06466839 | 11 | 1.29E+08 | − | OpenSea | RICS | NM_001142685 | TSS200 | 183 | ARHGAP32 |
| cg02078370 | 7 | 1.27E+08 | + | Island | | | | 22232 | ZNF800 |
| cg03163246 | 4 | 74921023 | + | OpenSea | PPBPL2 | NM_026769 | Body | 0 | PPBPP2 |
| cg21679294 | 11 | 1.02E+08 | − | Island | BIRC2; BIRC2 | NM_001166; | 1stExon; | 0 | BIRC2 |
| | | | | | | NM_001166 | 5′UTR | | |
| cg08872891 | 8 | 1.46E+08 | − | N_Shore | | | | 1942 | C8orf33 |
| cg20567895 | 1 | 95149580 | + | OpenSea | | | | 136316 | SLC44A3 |
| cg01673669 | 15 | 60298900 | + | S_Shore | | | | 0 | FOXB1 |
| cg12344605 | 17 | 11924919 | − | S_Shore | MAP2K4 | NM_003010 | Body | 0 | MAP2K4 |
| cg25297849 | 2 | 2.2E+08 | + | Island | IHH | NM_002181 | Body | 0 | IHH |
| cg03742273 | 1 | 2772664 | + | Island | | | | 66433 | TTC34 |
| cg12531953 | 8 | 56793075 | + | Island | LYN; LYN | NM_002350; | 5′UTR; | 0 | LYN |
| | | | | | | NM_001111097 | 5′UTR | | |
| cg23768117 | 17 | 79134486 | + | S_Shore | AATK | NM_001080395 | Body | 0 | AATK |
| cg01225095 | 12 | 32909323 | + | S_Shore | YARS2 | NM_001040436 | TSS1500 | 435 | YARS2 |
| cg15456476 | 16 | 85146180 | + | OpenSea | FAM92B | NM_198491 | TSS200 | 65 | FAM92B |
| cg08928569 | 16 | 3359325 | + | S_Shelf | ZNF75A | NM_153028 | 5′UTR | 0 | ZNF75A |
| cg00760321 | 1 | 32671490 | − | Island | IQCC; IQCC | NM_018134; | Body; | 0 | IQCC |
| | | | | | | NM_001160042 | 1stExon | | |
| cg05233877 | 6 | 31599162 | − | N_Shore | BAT2 | NM_080686 | Body | 0 | PRRC2A |
| cg12796916 | 11 | 67471698 | − | OpenSea | | | | 23012 | ALDH3B2 |
| cg04080595 | 1 | 2985649 | + | Island | PRDM16; | NM_022114; | TSS200; | 91 | PRDM16 |
| | | | | | FLJ42875; | NM_015440; | TSS1500; | | |
| cg20808462 | 4 | 2243252 | − | Island | HAUS3 | NM_024511 | 5′UTR | 0 | POLN |

1. A method of measuring DNA methylation in a biological sample obtained from a subject, the method comprising:
(a) generating an irritable bowel syndrome (IBS) inflammatory bowel disease (IBD) methylation profile from the biological sample obtained from the subject, wherein the profile comprises at least 50 CpG sites of the IBS/IBD biomarker genes listed in Tables 16-20; and
(b) measuring the amount of methylation in the IBS/IBD biomarker genes;
wherein the amount of biomarker methylation is used to classify the profile.

2. The method of claim 1, wherein the subject has manifested clinical symptoms associated with IBS.

3. The method of claim 1, wherein the subject has manifested clinical symptoms associated with IBD.

4. The method of claim 1, wherein the methylation profile comprises at least 100 of the CpG sites listed in Table 16, Table 17, Table 18, Table 19, or Table 20.

5. The method of claim 1, wherein generating the IBS/IBD methylation profile comprises preprocessing the biological sample with a kit for measuring the amount of methylation on all CpG sites.

6. The method of claim 1, wherein the IBS/IBD biomarker genes are selected from genes differentially methylated between IBS and healthy controls and listed in Table 16.

7. The method of claim 1, wherein the IBS/IBD biomarker genes are selected from genes differentially methylated between ulcerative colitis (UC) and healthy controls as shown in Table 17.

8. The method of claim 1, wherein the IBS/IBD biomarker genes are selected from genes differentially methylated between Crohn's Disease (CD) and healthy controls and listed in Table 18.

9. The method of claim 1, wherein a computer algorithm determines a conditional probability of IBS based on the profile.

10. The method of claim 1, wherein the IBS/IBD biomarker genes are selected from genes differentially methylated between IBS and IBD and listed in Table 19.

11. The method of claim 1, further comprising calculating the percentage of CpG sites on the IBS/IBD biomarker genes that are methylated, wherein a percentage of CpG sites methylated in excess of 40% is indicative of IBS or IBD.

12. A method of treating IBS comprising performing the method of claim 1, and administering treatment for IBS.

13. The method of claim 12, wherein the treatment comprises administering rifaximin, loperamide, eluxadoline, alosetron, lubiprostone, linaclotide, plecanatide, a laxative, an antihistamine, an antispasmodic, a neuromodulator, dietary therapy, or behavioral therapy.

14. The method of claim 1, further comprising:
(c) classifying the profile as:
(i) an IBS profile if at least 50% of the CpG sited on the genes listed in Table 16 are methylated;
(ii) a UC profile if at least 50% of the CpG sited on the genes listed in Table 17 are methylated; and/or
(iii) a CD profile if at least 50% of the CpG sited on the genes listed in Table 18 are methylated; and
(d) administering treatment for IBS, UC, or CD, in accordance with the classified profile.

15. A method of screening for IBS, UC, or CD in a subject, the method comprising:
(a) generating an IBS/BD methylation profile from a biological sample obtained from the subject, wherein the profile comprises at least 50 CpG sites of the IBS/IBD biomarker genes listed in Tables 16-20; and
(b) measuring the amount of methylation in the IBS/IBD biomarker genes;
wherein the amount of biomarker methylation is used to classify the profile, and a subject is identified as having IBS, UC, or CD based on the profile.

16. The method of claim 1, wherein the biological sample comprises blood, plasma, serum, or mucosal tissue.

17. The method of claim 16, wherein the sample is peripheral blood mononuclear cells (PBMCs), peripheral blood lymphocytes (PBL), or whole blood.

18. The method of claim 1, wherein the amount of biomarker methylation is greater than 54% of CpG sites on the IBS/IBD biomarker genes.
